SEQUENCE LISTING (188 sequences)

SEQ ID NO: 1 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 2 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTRFDHVVGNVPEMAPVAAYIKEFMGFHDFAEFTAEDVGTVESGLNSVVLANNSEAVLLPLNEPVHGTKRRSQIQTYLDYHGGPGVQHIALASSDVLRTLREMRARTAMGGFEFMAPPQPKYYEGVRRLAGDVLSEAQIKECQELGVLVDRDDQGVLLQIFTKPVGDRPTFFLEMIQRIGCMEKNETGQEYQKGGCGGFGKGNFSELFKSIEDYEKSLEAKQSAVAQ

SEQ ID NO: 3 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTRFDHVVGNVPEMAPVAAYMKGFTGFHEFAEFTAEDVGTAESGLNSVVLANNSEAVLLPLNEPVHGTKRRSQIQTYLDYHGGPGVQHIALASSDVLRTLREMRARSAMGGFEFMAPPQAKYYEGVRRLAGDVLSEAQIKECQELGVLVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 4 - protein, 439 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 5 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHIVGNVPEMAPVIDYMKGFLGFHEFAEFTAEDVGTTESGLNSVALANNSEAVLLPLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 6 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTRFDHIVGNVPEMAPVAAYIKEFMGFHDFAEFTAEDVGTVESGLNSVALANNSEAVLLPLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 7 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTRFDHIVGNVPEMAPVAAYMKGFTGFHEFAEFTAEDVGTAESGLNSVALANNSEAVLLPLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 8 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHIVGNVPEMAPVIDYMKGFLGFHEFAEFTAEDVGTTESGLNSVALANNSEAVLLPLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFAKGNFSELFKSIEDYEKSLEVKQSVVAQKS

SEQ ID NO: 9 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTRFDHIVGNVPEMAPVAAYIKEFMGFHDFAEFTAEDVGTVESGLNSVALANNSEAVLLPLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFAKGNFSELFKSIEDYEKSLEAKQSAVAQ

SEQ ID NO: 10 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTRFDHIVGNVPEMAPVAAYMKGFTGFHEFAEFTAEDVGTAESGLNSVALANNSEAVLLPLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFAKGNFSELFKSIEDYEKSLEAKQSGVAQQS

SEQ ID NO: 11 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHIVGNVPEMAPVIDYMKGFLGFHEFAEFTAEDVGTTESGLNSVTLANNSEAVLLPLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 12 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTRFDHIVGNVPEMAPVAAYIKEFMGFHDFAEFTAEDVGTVESGLNSVTLANNSEAVLLPLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 13 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTRFDHIVGNVPEMAPVAAYMKGFTGFHEFAEFTAEDVGTAESGLNSVTLANNSEAVLLPLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 14 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHIVGNVPEMAPVIDYMKGFLGFHEFAEFTAEDVGTTESGLNSVVLANNSEAVLLNLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 15 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTRFDHIVGNVPEMAPVAAYIKEFMGFHDFAEFTAEDVGTVESGLNSVVLANNSEAVLLNLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 16 - protein, 438 aa, Alopecurus myosuroides; identical to SEQ ID NO: 15

SEQ ID NO: 17 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVALANNSEAVLLPLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 18 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVALANNSEAVLLPLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 19 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVALANNSEAVLLPLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 20 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMRPPQAKYYEGVRREAGDVLSEEQIKECQELGVYVDRDDQGVLLQIFTKPVGDRPTFFLEMIQRIGCMEKDEVGQEYQKGGCGGFAKGNFSELFKSIEDYEKSLEVKQSVVAQKS

SEQ ID NO: 21 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVVLANNSEAVLLPLNEPVHGTKRRSQIQTYLDYHGGPGVQHIALASSDVLRTLREMRARTAMGGFEFMRPPQPKYYEGVRREAGDVLSEAQIKECQELGVYVDRDDQGVLLQIFTKPVGDRPTFFLEMIQRIGCMEKNETGQEYQKGGCGGFAKGNFSELFKSIEDYEKSLEAKQSAVAQ

SEQ ID NO: 22 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVVLANNSEAVLLPLNEPVHGTKRRSQIQTYLDYHGGPGVQHIALASSDVLRTLREMRARSAMGGFEFMRPPQAKYYEGVRREAGDVLSEAQIKECQELGVYVDRDDQGVLLQIFTKPVGDRPTFFLEMIQRIGCMEKDEIGQEYQKGGCGGFAKGNFSELFKSIEDYEKSLEAKQSGVAQQS

SEQ ID NO: 23 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVALANNSEAVLLPLNEPVHGTKRRSQIQTYLEYHGGPGVQHIALASNDVLRTLREMRARTPMGGFEFMRPPQAKYYEGVRREAGDVLSEEQIKECQELGVYVDRDDQGVLLQIFTKPVGDRPTFFLEMIQRIGCMEKDEVGQEYQKGGCGGFAKGNFSELFKSIEDYEKSLEVKQSVVAQKS

SEQ ID NO: 24 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVALANNSEAVLLPLNEPVHGTKRRSQIQTYLDYHGGPGVQHIALASSDVLRTLREMRARTAMGGFEFMRPPQPKYYEGVRREAGDVLSEAQIKECQELGVYVDRDDQGVLLQIFTKPVGDRPTFFLEMIQRIGCMEKNETGQEYQKGGCGGFAKGNFSELFKSIEDYEKSLEAKQSAVAQ

SEQ ID NO: 25 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVALANNSEAVLLPLNEPVHGTKRRSQIQTYLDYHGGPGVQHIALASSDVLRTLREMRARSAMGGFEFMRPPQAKYYEGVRREAGDVLSEAQIKECQELGVYVDRDDQGVLLQIFTKPVGDRPTFFLEMIQRIGCMEKDEIGQEYQKGGCGGFAKGNFSELFKSIEDYEKSLEAKQSGVAQQS

SEQ ID NO: 26 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVALANNSEAVLLPLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFAKGNFSELFKSIEDYEKSLEVKQSVVAQKS

SEQ ID NO: 27 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVALANNSEAVLLPLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFAKGNFSELFKSIEDYEKSLEAKQSAVAQ

SEQ ID NO: 28 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVALANNSEAVLLPLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFAKGNFSELFKSIEDYEKSLEAKQSGVAQQS

SEQ ID NO: 29 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVALANNSEAVLLNLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFAKGNFSELFKSIEDYEKSLEVKQSVVAQKS

SEQ ID NO: 30 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVALANNSEAVLLNLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFAKGNFSELFKSIEDYEKSLEAKQSAVAQ

SEQ ID NO: 31 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVALANNSEAVLLNLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFAKGNFSELFKSIEDYEKSLEAKQSGVAQQS

SEQ ID NO: 32 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVVLANNSEAVLLNLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 33 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVVLANNSEAVLLNLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 34 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVVLANNSEAVLLNLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 35 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVALANNSEAVLLNLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 36 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVALANNSEAVLLNLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 37 - protein, 440 aa, Alopecurus myosuroides; identical to SEQ ID NO: 19

SEQ ID NO: 38 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVALANNSEAVLLNLNEPVHGTKRRSQIQTYLEYHGGPGVQHIALASNDVLRTLREMRARTPMGGFEFMRPPQAKYYEGVRREAGDVLSEEQIKECQELGVYVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 39 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVALANNSEAVLLNLNEPVHGTKRRSQIQTYLDYHGGPGVQHIALASSDVLRTLREMRARTAMGGFEFMRPPQPKYYEGVRREAGDVLSEAQIKECQELGVYVDRDDQGVLLQIFTKPVGDRPTFFLEMIQRIGCMEKNETGQEYQKGGCGGFGKGNFSELFKSIEDYEKSLEAKQSAVAQ

SEQ ID NO: 40 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVALANNSEAVLLNLNEPVHGTKRRSQIQTYLDYHGGPGVQHIALASSDVLRTLREMRARSAMGGFEFMRPPQAKYYEGVRREAGDVLSEAQIKECQELGVYVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 41 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVTLANNSEAVLLNLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFAKGNFSELFKSIEDYEKSLEVKQSVVAQKS

SEQ ID NO: 42 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVTLANNSEAVLLNLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFAKGNFSELFKSIEDYEKSLEAKQSAVAQ

SEQ ID NO: 43 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVTLANNSEAVLLNLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFAKGNFSELFKSIEDYEKSLEAKQSGVAQQS

SEQ ID NO: 44 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVALANNSEAVLLNLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQNGGCGGFGKGNFSELFKSIEDYEKSLEVKQSVVAQKS

SEQ ID NO: 45 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVALANNSEAVLLNLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQNGGCGGFGKGNFSELFKSIEDYEKSLEAKQSAVAQ

SEQ ID NO: 46 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVALANNSEAVLLPLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQNGGCGGFGKGNFSELFKSIEDYEKSLEAKQSGVAQQS

SEQ ID NO: 47 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHIVGNVPEMAPVIDYMKGFLGFHEFAEFTAEDVGTTESGLNSVVLANNSEAVLLNLNEPVHGTKRRSQIQTYLEYHGGPGVQHIALATNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 48 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTRFDHIVGNVPEMAPVAAYIKEFMGFHDFAEFTAEDVGTVESGLNSVVLANNSEAVLLNLNEPVHGTKRRSQIQTYLDYHGGPGVQHIALATSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 49 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTRFDHIVGNVPEMAPVAAYMKGFTGFHEFAEFTAEDVGTAESGLNSVVLANNSEAVLLNLNEPVHGTKRRSQIQTYLDYHGGPGVQHIALATSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 50 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVVLANNSEAVLLPLNEPVHGTKRRSQIQTYLEYHGGPGVQHIALATNDVLRTLREMRARTPMGGFEFMRPPQAKYYEGVRREAGDVLSEEQIKECQELGVYVDRDDQGVLLQIFTKPVGDRPTFFLEMIQRIGCMEKDEVGQEYQKGGCGGFAKGNFSELFKSIEDYEKSLEVKQSVVAQKS

SEQ ID NO: 51 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVVLANNSEAVLLPLNEPVHGTKRRSQIQTYLDYHGGPGVQHIALATSDVLRTLREMRARTAMGGFEFMRPPQPKYYEGVRREAGDVLSEAQIKECQELGVYVDRDDQGVLLQIFTKPVGDRPTFFLEMIQRIGCMEKNETGQEYQKGGCGGFAKGNFSELFKSIEDYEKSLEAKQSAVAQ

SEQ ID NO: 52 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVVLANNSEAVLLPLNEPVHGTKRRSQIQTYLDYHGGPGVQHIALATSDVLRTLREMRARSAMGGFEFMRPPQAKYYEGVRREAGDVLSEAQIKECQELGVYVDRDDQGVLLQIFTKPVGDRPTFFLEMIQRIGCMEKDEIGQEYQKGGCGGFAKGNFSELFKSIEDYEKSLEAKQSGVAQQS

SEQ ID NO: 53 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVVLANNSEAVLLNLNEPVHGTKRRSQIQTYLEYHGGPGVQHIALATNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 54 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVVLANNSEAVLLNLNEPVHGTKRRSQIQTYLDYHGGPGVQHIALATSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 55 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVVLANNSEAVLLNLNEPVHGTKRRSQIQTYLDYHGGPGVQHIALATSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 56 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTGFDHIVGNVPEMAPVIDYMKGFLGFHEFAEFTAEDVGTTESGLNSVALANNSEAVLLNLNEPVHGTKRRSQIQTYLEYHGGPGVQHIALATNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 57 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTGFDHIVGNVPEMAPVAAYIKEFMGFHDFAEFTAEDVGTVESGLNSVALANNSEAVLLNLNEPVHGTKRRSQIQTYLDYHGGPGVQHIALATSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 58 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTGFDHIVGNVPEMAPVAAYMKGFTGFHEFAEFTAEDVGTAESGLNSVALANNSEAVLLNLNEPVHGTKRRSQIQTYLDYHGGPGVQHIALATSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 59 - protein, 10 aa, Artificial
  Protein motif
  MISC_FEATURE (2)..(2): Tyr or Phe
  MISC_FEATURE (3)..(3): Gly
  MISC_FEATURE (4)..(4): Leu or Ile
  MISC_FEATURE (5)..(5): Thr, Gln, Ser, or Arg
  MISC_FEATURE (6)..(6): Gly
  MISC_FEATURE (7)..(7): Phe or Leu
  MISC_FEATURE (8)..(8): Asp
  MISC_FEATURE (9)..(9): His
  MISC_FEATURE (10)..(10): Val, Ala, Ile, or Cys
RXXXXXXXXX

SEQ ID NO: 60 - protein, 10 aa, Artificial
  Protein motif
  MISC_FEATURE (2)..(2): Leu
  MISC_FEATURE (3)..(3): Asn
  MISC_FEATURE (4)..(4): Ser
  MISC_FEATURE (5)..(5): Val, Ala or Met
  MISC_FEATURE (6)..(6): Ala or Thr
  MISC_FEATURE (7)..(7): Leu
  MISC_FEATURE (8)..(8): Ala
  MISC_FEATURE (9)..(9): Asn, Ser or Cys
  MISC_FEATURE (10)..(10): Asn or Thr
VXXXXXXXXX

SEQ ID NO: 61 - protein, 10 aa, Artificial
  protein motif
  MISC_FEATURE (2)..(2): Ala ,Met, Gly, Arg, Asn, or Thr
  MISC_FEATURE (3)..(3): Val
  MISC_FEATURE (4)..(4): Leu or Pro
  MISC_FEATURE (5)..(5): Leu, Ile or Phe
  MISC_FEATURE (6)..(6): Asn
  MISC_FEATURE (7)..(7): Leu, Met, Ile or Val
  MISC_FEATURE (8)..(8): Asn
  MISC_FEATURE (9)..(9): Glu
  MISC_FEATURE (10)..(10): Glu
PXXXXXXXXX

SEQ ID NO: 62 - protein, 10 aa, Artificial
  protein motif
  MISC_FEATURE (2)..(2): Ile, Leu or Met
  MISC_FEATURE (3)..(3): Ala
  MISC_FEATURE (4)..(4): Leu or Val
  MISC_FEATURE (5)..(5): Ala, Met, Lys, Leu, Ser or Val
  MISC_FEATURE (6)..(6): Thr
  MISC_FEATURE (7)..(7): Ser, Glu, Asn, Asp, His or Arg
  MISC_FEATURE (8)..(8): Asp, Glu or Asn
  MISC_FEATURE (9)..(9): Val or Ile
  MISC_FEATURE (10)..(10): Phe, Ile or Leu
SXXXXXXXXX

SEQ ID NO: 63 - protein, 10 aa, Artificial
  Protein motif
  MISC_FEATURE (2)..(2): Gln, Glu, Thr or Lys
  MISC_FEATURE (3)..(3): Glu, Ala, Met or Val
  MISC_FEATURE (4)..(4): Tyr, Gln or Ala
  MISC_FEATURE (5)..(5): Gln, Gly or Ala
  MISC_FEATURE (6)..(6): Asn
  MISC_FEATURE (7)..(7): Gly
  MISC_FEATURE (8)..(8): Gln, Cys, Gly or Ala
  MISC_FEATURE (9)..(9): Cys or Gly
  MISC_FEATURE (10)..(10): Gly or Leu
KXXXXXXXXX

SEQ ID NO: 64 - DNA, 1320 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacgtcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatggcgccaccgcaggcgaaatactatgaaggcgtgcggcgcatcgcaggtgacgtgctctcggaagagcagatcaaggaatgccaggagctgggggtgctagtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 65 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 66 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactaggttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 67 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactagattcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 68 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgcgaagggcaatttctccgagctgttcaagtccattgaggactatgagaaatcccttgaggtcaagcaatctgttgtagctcagaaatcctag

SEQ ID NO: 69 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactaggttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcgctaagggtaacttctcagaacttttcaagtctattgaagattacgaaaagtcattggaggctaagcagtctgcagttgctcagtga

SEQ ID NO: 70 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactagattcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcgcgaagggtaacttctcagaattgtttaagtcaatcgaggattacgaaaagtctcttgaggctaagcagtctggagtggctcagcagtcttga

SEQ ID NO: 71 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtgacgctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 72 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactaggttcgaccacatcgttggtaatgtgcctgagatggctccagtggcagcatacatcaaggaattcatgggtttccacgatttcgctgagttcactgcagaggatgttggtactgtggagtctggattgaactcagtgacgcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 73 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactagattcgatcacatcgttggtaacgtgccagagatggctcctgtggctgcttacatgaagggattcactggattccacgagttcgcagagtttacagctgaggacgtgggtacagctgagtcaggtcttaactctgtgacgcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 74 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgaacctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 75 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactaggttcgaccacatcgttggtaatgtgcctgagatggctccagtggcagcatacatcaaggaattcatgggtttccacgatttcgctgagttcactgcagaggatgttggtactgtggagtctggattgaactcagtggtgcttgctaacaattcagaggctgtgcttttgaacttgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 76 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactagattcgatcacatcgttggtaacgtgccagagatggctcctgtggctgcttacatgaagggattcactggattccacgagttcgcagagtttacagctgaggacgtgggtacagctgagtcaggtcttaactctgtggtgcttgctaacaactctgaggctgtgttgttgaacttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 77 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 78 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggattcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 79 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 80 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgtacgtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgcgaagggcaatttctccgagctgttcaagtccattgaggactatgagaaatcccttgaggtcaagcaatctgttgtagctcagaaatcctag

SEQ ID NO: 81 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggtgcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgtttacgttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcgctaagggtaacttctcagaacttttcaagtctattgaagattacgaaaagtcattggaggctaagcagtctgcagttgctcagtga

SEQ ID NO: 82 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggtgcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgtacgtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcgcgaagggtaacttctcagaattgtttaagtcaatcgaggattacgaaaagtctcttgaggctaagcagtctggagtggctcagcagtcttga

SEQ ID NO: 83 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgtacgtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgcgaagggcaatttctccgagctgttcaagtccattgaggactatgagaaatcccttgaggtcaagcaatctgttgtagctcagaaatcctag

SEQ ID NO: 84 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgtttacgttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcgctaagggtaacttctcagaacttttcaagtctattgaagattacgaaaagtcattggaggctaagcagtctgcagttgctcagtga

SEQ ID NO: 85 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgtacgtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcgcgaagggtaacttctcagaattgtttaagtcaatcgaggattacgaaaagtctcttgaggctaagcagtctggagtggctcagcagtcttga

SEQ ID NO: 86 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgcgaagggcaatttctccgagctgttcaagtccattgaggactatgagaaatcccttgaggtcaagcaatctgttgtagctcagaaatcctag

SEQ ID NO: 87 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcgctaagggtaacttctcagaacttttcaagtctattgaagattacgaaaagtcattggaggctaagcagtctgcagttgctcagtga

SEQ ID NO: 88 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcgcgaagggtaacttctcagaattgtttaagtcaatcgaggattacgaaaagtctcttgaggctaagcagtctggagtggctcagcagtcttga

SEQ ID NO: 89 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgaacctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgcgaagggcaatttctccgagctgttcaagtccattgaggactatgagaaatcccttgaggtcaagcaatctgttgtagctcagaaatcctag

SEQ ID NO: 90 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgaacttgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcgctaagggtaacttctcagaacttttcaagtctattgaagattacgaaaagtcattggaggctaagcagtctgcagttgctcagtga

SEQ ID NO: 91 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgaacttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcgcgaagggtaacttctcagaattgtttaagtcaatcgaggattacgaaaagtctcttgaggctaagcagtctggagtggctcagcagtcttga

SEQ ID NO: 92 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgaacctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 93 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggtgcttgctaacaattcagaggctgtgcttttgaacttgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 94 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggtgcttgctaacaactctgaggctgtgttgttgaacttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 95 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgaacctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 96 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgaacttgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 97 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgaacttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 98 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgaacctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgtacgtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 99 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgaacttgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgtttacgttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 100 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgaacttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgtacgtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 101 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtgacgctcgccaacaactccgaggccgtgctgctgaacctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgcgaagggcaatttctccgagctgttcaagtccattgaggactatgagaaatcccttgaggtcaagcaatctgttgtagctcagaaatcctag

SEQ ID NO: 102 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtgacgcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcgctaagggtaacttctcagaacttttcaagtctattgaagattacgaaaagtcattggaggctaagcagtctgcagttgctcagtga

SEQ ID NO: 103 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtgacgcttgctaacaactctgaggctgtgttgttgaacttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcgcgaagggtaacttctcagaattgtttaagtcaatcgaggattacgaaaagtctcttgaggctaagcagtctggagtggctcagcagtcttga

SEQ ID NO: 104 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgaacctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaacggtggctgcggcgggtttggcaagggcaatttctccgagctgttcaagtccattgaggactatgagaaatcccttgaggtcaagcaatctgttgtagctcagaaatcctag

SEQ ID NO: 105 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgaacttgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaacggtggttgtggaggtttcggcaagggtaacttctcagaacttttcaagtctattgaagattacgaaaagtcattggaggctaagcagtctgcagttgctcagtga

SEQ ID NO: 106 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgaacttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaacggtggttgcggtggtttcggaaagggtaacttctcagaattgtttaagtcaatcgaggattacgaaaagtctcttgaggctaagcagtctggagtggctcagcagtcttga

SEQ ID NO: 107 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgaacctcaacgagcccgtgcacggcacaaagcgacggagccagatacagacgtacctggagtatcacggcgggcccggcgtgcagcacatcgcgctcgccactaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 108 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactaggttcgaccacatcgttggtaatgtgcctgagatggctccagtggcagcatacatcaaggaattcatgggtttccacgatttcgctgagttcactgcagaggatgttggtactgtggagtctggattgaactcagtggtgcttgctaacaattcagaggctgtgcttttgaacttgaacgagcctgtgcacggtactaaaaggagatcacagattcaaacttaccttgattaccacggaggtcctggagttcagcacatcgcattggctacatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 109 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactagattcgatcacatcgttggtaacgtgccagagatggctcctgtggctgcttacatgaagggattcactggattccacgagttcgcagagtttacagctgaggacgtgggtacagctgagtcaggtcttaactctgtggtgcttgctaacaactctgaggctgtgttgttgaacttgaatgagccagtgcacggtactaagagaaggtcacaaattcagacatacttggattaccacggtggtcctggtgtgcagcacatcgctttggcaacttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 110 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgccgctcaacgagcccgtgcacggcacaaagcgacggagccagatacagacgtacctggagtatcacggcgggcccggcgtgcagcacatcgcgctcgccactaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgtacgtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgcgaagggcaatttctccgagctgttcaagtccattgaggactatgagaaatcccttgaggtcaagcaatctgttgtagctcagaaatcctag

SEQ ID NO: 111 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggtgcttgctaacaattcagaggctgtgcttttgccattgaacgagcctgtgcacggtactaaaaggagatcacagattcaaacttaccttgattaccacggaggtcctggagttcagcacatcgcattggctacatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgtttacgttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcgctaagggtaacttctcagaacttttcaagtctattgaagattacgaaaagtcattggaggctaagcagtctgcagttgctcagtga

SEQ ID NO: 112 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggtgcttgctaacaactctgaggctgtgttgttgccattgaatgagccagtgcacggtactaagagaaggtcacaaattcagacatacttggattaccacggtggtcctggtgtgcagcacatcgctttggcaacttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgtacgtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcgcgaagggtaacttctcagaattgtttaagtcaatcgaggattacgaaaagtctcttgaggctaagcagtctggagtggctcagcagtcttga

SEQ ID NO: 113 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgaacctcaacgagcccgtgcacggcacaaagcgacggagccagatacagacgtacctggagtatcacggcgggcccggcgtgcagcacatcgcgctcgccactaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 114 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggtgcttgctaacaattcagaggctgtgcttttgaacttgaacgagcctgtgcacggtactaaaaggagatcacagattcaaacttaccttgattaccacggaggtcctggagttcagcacatcgcattggctacatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 115 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggtgcttgctaacaactctgaggctgtgttgttgaacttgaatgagccagtgcacggtactaagagaaggtcacaaattcagacatacttggattaccacggtggtcctggtgtgcagcacatcgctttggcaacttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 116 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgggattcgaccacatcgtgggcaacgtcccggagatggccccggtcatagactacatgaaaggcttcttggggttccacgagttcgccgagttcaccgccgaggacgtgggcacgaccgagagcgggctcaactcggtggctctcgccaacaactccgaggccgtgctgctgaacctcaacgagcccgtgcacggcacaaagcgacggagccagatacagacgtacctggagtatcacggcgggcccggcgtgcagcacatcgcgctcgccactaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 117 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactggcttcgaccacatcgttggtaatgtgcctgagatggctccagtggcagcatacatcaaggaattcatgggtttccacgatttcgctgagttcactgcagaggatgttggtactgtggagtctggattgaactcagtggctcttgctaacaattcagaggctgtgcttttgaacttgaacgagcctgtgcacggtactaaaaggagatcacagattcaaacttaccttgattaccacggaggtcctggagttcagcacatcgcattggctacatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 118 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactggcttcgatcacatcgttggtaacgtgccagagatggctcctgtggctgcttacatgaagggattcactggattccacgagttcgcagagtttacagctgaggacgtgggtacagctgagtcaggtcttaactctgtggctcttgctaacaactctgaggctgtgttgttgaacttgaatgagccagtgcacggtactaagagaaggtcacaaattcagacatacttggattaccacggtggtcctggtgtgcagcacatcgctttggcaacttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 119 - DNA, 13642 bp, Artificial
  expression vector pBin Vector Avena Sativa HPPD V207
agcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcttacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaattacacatatgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtgacgctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctagggatccccgggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcctagagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcggcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgcttcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctctagga

SEQ ID NO: 120 - DNA, 13642 bp, Artificial
  expression vector pBin Vector Avena Sativa HPPD V208
agcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcttacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaattacacatatgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgaacctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctagggatccccgggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcctagagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcggcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgcttcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctctagga

SEQ ID NO: 121 - DNA, 13642 bp, Artificial
  expression vector pBin Vector Avena Sativa HPPD V208
agcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcttacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaattacacatatgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacggggttcgaccacatcgtgggcaacgtcccggagatggccccggtcatagactacatgaaaggcttcttggggttccacgagttcgccgagttcaccgccgaggacgtgggcacgaccgagagcgggctcaactcggtggccctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctagggatccccgggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcctagagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcggcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgcttcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctctagga

SEQ ID NO: 122 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHIVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 123 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTRFDHIVGNVPEMAPVAAYIKEFMGFHDFAEFTAEDVGTVESGLNSVVLANNSEAVLLPLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFGKGNFSELFKSIEDYEKSLEAKQSAVAQ

SEQ ID NO: 124 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHIVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMRPPQAKYYEGVRREAGDVLSEEQIKECQELGVMVDRDDQGVLLQIFTKPVGDRPTFFLEMIQRIGCMEKDEVGQEYQKGGCGGFAKGNFSELFKSIEDYEKSLEVKQSVVAQKS

SEQ ID NO: 125 - protein, 438 aa, Apera spica-venti
MPPTTATATGAAAAAVTPEHAARRFPRVVRINPRSDRFPVLAFHHVEFWCADAASAAGRFSFALGAPLAARSDLSTGNSAHASHLLRSGALAFLFTAPYAPPPQDAANAAATASIPSFSAEAARTFSAAHGLAVRSVAVRVADAAEAFRTSVAAGARPAFAPADLGRGFGLAEVELYGDVVLRFVSHPDGDDVPFLPGFEGVSRPGAVDYGLTRFDHIVGNVPEMAPVAAYIKEFMGFHDFAEFTAEDVGTVESGLNSVVLANNSEAVLLPLNEPVHGTKRRSQIQTYLDYHGGPGVQHIALASSDVLRTLREMRARTAMGGFEFMRPPQPKYYEGVRREAGDVLSEAQIKECQELGVMVDRDDQGVLLQIFTKPVGDRPTFFLEMIQRIGCMEKNETGQEYQKGGCGGFAKGNFSELFKSIEDYEKSLEAKQSAVAQ

SEQ ID NO: 126 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTRFDHIVGNVPEMAPVAAYMKGFTGFHEFAEFTAEDVGTAESGLNSVVLANNSEAVLLPLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFGKGNFSELFKSIEDYEKSLEAKQSGVAQQS

SEQ ID NO: 127 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSADAARTFSAAHGLAVRSVAIRVADAAEAFHTSVAGGARPAFAPADLGSGFGLAEVELYGDVVLRFVSHPDGDDVPFLPGFEGVSRPGAVDYGLTRFDHIVGNVPEMAPVAAYMKGFTGFHEFAEFTAEDVGTAESGLNSVVLANNSEAVLLPLNEPVHGTKRRSQIQTYLDYHGGPGVQHIALASSDVLRTLREMRARSAMGGFEFMRPPQAKYYEGVRREAGDVLSEAQIKECQELGVMVDRDDQGVLLQIFTKPVGDRPTFFLEMIQRIGCMEKDEIGQEYQKGGCGGFAKGNFSELFKSIEDYEKSLEAKQSGVAQQS

SEQ ID NO: 128 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 129 - DNA, 1317 bp, Apera spica-venti
atgcctccaactacagctactgctacaggtgctgctgctgctgcagttacacctgagcacgctgctagaaggtttccaagagtggtgaggatcaacccaaggtctgacaggttcccagtgttggcattccaccacgttgagttctggtgcgcagatgcagcttctgctgctggtaggttctctttcgcacttggagctcctcttgctgctaggtcagacttgtctactggtaactctgctcacgcttcacaccttcttaggtctggtgctttggctttccttttcactgctccttatgctcctccacctcaagatgctgctaacgcagctgctacagcttctatcccttctttctcagctgaggcagctaggactttctctgctgctcatggacttgcagttaggtctgtggctgtgagagttgctgatgcagctgaggctttcaggacttctgtggctgcaggtgcaagacctgcttttgctcctgctgatttgggaagaggattcggtcttgctgaggtggagttgtacggagacgttgtgttgaggttcgtgtctcatccagatggagatgacgtgcctttccttcctggattcgagggtgtgtctaggcctggtgctgtggattacggacttactaggttcgaccacatcgttggtaatgtgcctgagatggctccagtggcagcatacatcaaggaattcatgggtttccacgatttcgctgagttcactgcagaggatgttggtactgtggagtctggattgaactcagtggtgcttgctaacaattcagaggctgtgcttttgccattgaacgagcctgtgcacggtactaaaaggagatcacagattcaaacttaccttgattaccacggaggtcctggagttcagcacatcgcattggcttcatcagacgtgttgaggacattgagggagatgagggctaggactgctatgggtggattcgagttcatgaggccacctcagccaaagtactacgagggtgttaggagggaggcaggtgatgttttgtcagaggctcaaatcaaggagtgccaggaacttggtgttatggttgacagggacgatcagggtgtgttgcttcagattttcacaaagcctgtgggtgacaggcctacttttttccttgagatgattcagaggattggatgcatggagaagaacgagactggtcaggagtaccagaagggtggttgtggaggtttcggcaagggtaacttctcagaacttttcaagtctattgaagattacgaaaagtcattggaggctaagcagtctgcagttgctcagtga

SEQ ID NO: 130 - DNA, 1323 bp, Avena sativa
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgccaagggcaatttctccgagctgttcaagtccattgaggactatgagaaatcccttgaggtcaagcaatctgttgtagctcagaaatcctag

SEQ ID NO: 131 - DNA, 1323 bp, Apera spica-venti
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacatcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatgcggccaccgcaggcgaaatactatgaaggcgtgcggcgcgaggcaggtgacgtgctctcggaagagcagatcaaggaatgccaggagctgggggtgatggtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttgctaagggcaatttctccgagctgttcaagtccattgaggactatgagaaatcccttgaggtcaagcaatctgttgtagctcagaaatcctag

SEQ ID NO: 132 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactagattcgatcacatcgttggtaacgtgccagagatggctcctgtggctgcttacatgaagggattcactggattccacgagttcgcagagtttacagctgaggacgtgggtacagctgagtcaggtcttaactctgtggtgcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcggaaagggtaacttctcagaattgtttaagtcaatcgaggattacgaaaagtctcttgaggctaagcagtctggagtggctcagcagtcttga

SEQ ID NO: 133 - DNA, 1323 bp, Alopecurus myosuroides
atgccacctacaactgctactgctactggagctgctgctgctgctgttacaccagagcatgctgcaaggagattcccaagagtggtgagggtgaacccaagatctgacaggttccctgttcttgctttccaccacgttgagttctggtgcgctgatgctgcatcagctgcaggtaggttctctttcgctttgggagctcctttggctgctaggtctgacttgtctacaggtaattcttctcacgcttcacaccttttgaggtctggagcattggcattccttttcacagctccttacgctcctcctccacaagatgctgctgatgcagctgctactgcttcaatcccttctttctcagctgacgctgctaggactttttctgctgctcacggacttgctgttaggtcagtggcaatcagggttgctgatgctgctgaggcattccacacttctgttgcaggtggagctagacctgctttcgctcctgctgacttgggttctggattcggacttgcagaggtggaattgtacggagacgtggtgttgaggttcgtttcacacccagatggagacgacgttcctttcttgcctggtttcgagggagtttcaaggcctggagcagtggattacggtttgactagattcgatcacatcgttggtaacgtgccagagatggctcctgtggctgcttacatgaagggattcactggattccacgagttcgcagagtttacagctgaggacgtgggtacagctgagtcaggtcttaactctgtggtgcttgctaacaactctgaggctgtgttgttgcctttgaatgagccagtgcacggtactaagagaaggtcacaaattcagacatacttggattaccacggtggtcctggtgtgcagcacatcgctttggcatcttctgacgtgcttaggactcttagggagatgagggctaggtcagctatgggaggattcgagttcatgaggcctcctcaggctaagtactatgagggagtgaggagagaggctggagatgttttgtctgaggcacagatcaaggagtgccaagagcttggtgtgatggtggatagggacgatcagggtgtgttgttgcagatttttacaaagcctgttggtgatagacctacatttttcttggaaatgatccagaggatcggttgcatggagaaggatgagattggtcaggagtaccagaagggtggttgcggtggtttcgcaaagggtaacttctcagaattgtttaagtcaatcgaggattacgaaaagtctcttgaggctaagcagtctggagtggctcagcagtcttga

SEQ ID NO: 134 - protein, 436 aa, Aegilops tauschii
MPPTPTTPAATGAGAAAAVTPEHAWPRRMVRFNPRSDRFHTLSFHHVEFWCADAASAAGRFAFALGAPLAARSDLSTGNSVHASQLLRSGNLAFLFTAPYANGCDAATASLPSFSADAARRFSADHGLAVRSIALRVADAAEAFRASVDGGARPAFSPVDLGRGFGFAEVELYGDVVLRFVSHPDGTDVPFLPGFEGVSNPGAVDYGLTRFDHVVGNVPELASAAAYVAGFTGFHEFAEFTTEDVGTAESGLNSMVLANNSEGVLLPLNEPVHGTKRRSQIQTFLEHHGGPGVQHIAVASSDVLRTLREMRARSAMGGFDFLPPPLPKYYEGVRRIAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 135 - protein, 440 aa, Ananas comosus
MGNAPEGETEGSGFKLRGFRGFVRANPRSDRFPVHGFHHVEFWTGDASTAAGRFAFALGMPLSARSDLSTGNPVHASYLLRSRSLRFLFSAPYGPGPGAGAAIPSFDPERARRFVAAHGLAVRAVALRVADAAAAFRVSVAHGARPAFPPADLGDGFALSEVELYGDVVLRYVSFPADQNPGEAPGFLPGFEDAAAAAAAGEPGRGPLDYGLRRLDHAVGNVPELGPAVAYVAGFTGFHEFAEFAAEDVGTAESGLNSVVLANNEETVLVPMNEPVHGTKRRSQIQTYLDHNGGPGVQHLALASDDVLRTLREMRARSGAGGFEFMPPPPPNYYDGVRRRAGDVLSDEQIAECKELGVLVDRDDQGVLLQIFTKPIGDRPTIFLEIIQRIGCMMKDDDGREYQKGGCGGFGKGNFSELFKSIEEYEKSLESKQSTTVPAS

SEQ ID NO: 136 - protein, 445 aa, Arabidopsis thaliana
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 137 - protein, 441 aa, Arabis nemorensis
MVHENAAVSETHQQNGAASPGFKLVGYSKFVRKNPKSDKFKVKRFHHIEFWCGDATNVARRFSWGLGMRFTAKSDLSTGNMVHASYLLTSGDLRFLFTAPYSPSLSAGESRPIATSPIPTFDHVSCRSFFSSHGLGVRAVAVEVEDAESAFSISVANGAIPSSPPILLDESVTIAEVKLYGDVVLRYVSHKGEVNTDFLPGFELVEDTSSFPLDYGIRRLDHAVGNVPELGPALTYIAGFTGFHQFAEFTADDIGTAESGLNSAVLASNDEMVLLPINEPVHGTKRKSQIQTYLEHNEGAGLQHLALMSEDIFRTLREMRKRSSVGGFDFMPSPPPTYYRNLKNRVGDVLSDEKIKECEELGILVDRDDQGTLLQIFTKPLGDRPTIFIEIIQRIGCMVKDEEGKVYQSGGCGGFGKGNFSELFKSIEEYEKTLEAKQLVG

SEQ ID NO: 138 - protein, 436 aa, Brachypodium distachyon
MPPPATTAAPAAAAVTPEHARPPRRVARVNPRSDRFSALSFHHVELWCADAASAAGRFSFALGAPPAARSDLSTGNSAHASILLRSGSLAFLFTAPYAPSPASDSAASIPSFSASAARQFTADHGGLAVRAVALRVSSASDAFHASVSAGARPSFPPADLGQGFALAEVELYGDVVLRFISHPDENTEIPFLPGFESVSNPGASTYGLTRFDHVVGNVPSLAPVAAYIAGFTGFHEFAEFTAEDVGTADSGLNSVVLANNSERVLLPLNEPVHGTKRRSQIQTYLDHHGGPGVQHIALASDDVLRTLREMRARSAMGGFEFLAPPPPNYYDGVRRRAGDVLSEAQIKECQELGVLVDRDDQGVLLQIFTKPVGDRPTLFLEMIQRIGCMEKDEIGQEQQKGGCGGFGKGNFSELFRSIEEYEKSLEAKQSAVVQES

SEQ ID NO: 139 - protein, 440 aa, Brasscia napus
MGHENAAVSESQHHDDAASAASPGFKLVGFSKFVRKNPKSDKFKVKRFHHIEFWCGDATNVARRFSWGLGMRFSAKSDLSTGNMVHASYLLTSGDLRFLFTAPYSPSLSAGETSTASIPSFDHVSCRSFFSSHGLGVRAVAIEVEDAESAFSISVANGAVPSSPPNVLNGAVTIAEVKLYGDVVLRYVSYHNGAVNFLPGFESVDDTSSFPLDYGIRRLDHAVGNVPELGPALTYLAGFTGFHPFAEFTADDVGTAESGLNSAVLANNDEMVLLPVNEPVHGTKRKSQIQTFLEHNEGAGLQHLALMSEDIFRTLREMRKRSGVGGFDFMPSPPPTYYKNLKKRIGDVLSDEQIRECEELGILVDRDDQGTLLQIFTKPLGDRPTIFIEIIQRVGCMKKDEEGKVYQSGGCGGFGKGNFSELFKSIEEYEKTLEAKQLVG

SEQ ID NO: 140 - protein, 444 aa, Brassica oleracea
MGHENAAVSENQHHDEAAATSASPGFKLVGFSKFVRKNPKSDKFKVKRFHHIEFWCGDATNVARRFSWGLGMRFSAKSDLSTGNMVHASYLLTSGDLRFLFTAPYSPSLSAGEIPPNTTASIPSFDDVTYRSFFSSHGLGVRAVAIEVEDAESAFSVSVSNGAVPSSPPIALNDAVTIAEVKLYGDVVLRYVSYKAVTSVFLPRFETVEDTSSFPLDYGIRRLDHAVGNVPELGPALTYLSRFTGFHQFAEFTADDVGTAESGLNSAVLANNDETVLLPVNEPVHGTKRKSQIQTYLEHNEGAGVQHLALMSEDIFKTLREMRKRSGVGGFDFMPSPPPTYYKNLKNRVGDVLSDEEIKECEELGILVDRDDQGTLLQIFTKPLGDRPTIFIEIIQRIGCMKKDEEGRVYQSGGCGGFGKGNFSELFKSIEEYEKTLEAKQLVG

SEQ ID NO: 141 - protein, 443 aa, Brassica rapa
MGHENAAVSESHQHDDAASAASPGFKLVGFSKFVRKNPKSDKFKVKRFHHIEFWCGDATNVARRFSWGLGMRFSAKSDLSTGNMVHASYLLTSGDLRFLFTAPYSPSLSAGETRTSATASIPSFDHVSCRSFFSSHGLGVRAVAIEVEDAESAFSISVANGAVPSSPPNVLNGAVTIAEVKLYGDVVLRYVSYHNGAVNFLPGFESVDDTSSFPLDYGIRRLDHAVGNVPELGPALTYLAGFTGFHQFAEFTADDVGTAESGLNSAVLASNDEMVLLPVNEPVHGTKRKSQIQTFLEHNEGAGLQHLALMSEDIFRTLREMRKRSGVGGFDFMPSPPPTYYKNLKKRVGDVLSDEQIRECEELGILVDRDDQGTLLQIFTKPLGDRPTIFIEIIQRVGCMKKDEEGKVYQSGGCGGFGKGNFSELFKSIEEYEKTLEAKQLVG

SEQ ID NO: 142 - protein, 434 aa, Bromus tectorum
MPPTPTTPAAAGAAAAAVATPPRRIVRVNPRSDRFDVLAFHHVEFWCADASSAAGRFAFGLGAPLAARSDLSTGNSAHASLLLRSGSLAFLFTAPYAHGCAADTASLPSFSADAARRFSADHGLAVRSVAIRVADAAEAFRASVDGGARPAFAPADLGSGFGLAEVELYGDVVLRFVSYPDEGATDVPFLPGFEAVSNPDAVDYGLTRFDHVVGNVPELAPAAAYVAGFTGFHEFAEFTAEDVGTTESGLNSVVLASNSEGVLLPLNEPVHGTKRRSQIQTFLEHHGGPGVQHIALASSDVLRTLREMRARSAMGGFDFLPPPLPKYYEGVRRLAGDVLSEAQIKECQELGVLVDRDDQGVLLQIFTKPVGDRPTLFLEMIQRIGCMEKDERGQEYQKGGCGGFGKGNFSELFKSIEDYEKSLEAKQSAAVQES

SEQ ID NO: 143 - protein, 450 aa, Camelina sativa
MGHQNAAVSDTQQHNGDDDSAAASSPGFKLVGFSNFVRKNPKSDKFKVKRFHHIEFWCGDATNVARRFSWGLGMRFSAKSDLSTGNMVHASYLLTSGDLRFLFTAPYSPSLTAGETKPTATASIPSFDHVSSRSFFSSHGLGVRAVAIEVEDAESAFSVSVSNGAIPSSPPMVLDGGAVTIAEVKLYGDVVLRYVSYKEGDTDKSEFLPGFEPVVDTSSFPLDYGIRRLDHAVGNVPELGPALTYIAGFTGFHQFAEFTADDVGTAESGLNSAVLASNDEMVLLPINEPVHGTKRKSQIQTYLEHNEGAGLQHLALMSEDIFRTLREMRKRSSVGGFDFMPSPPPTYYQNLKKRVGDVLSDEQIKECEELGILVDRDDQGTLLQIFTKPLGDRPTIFIEIIQRVGCMMKDDEGKAYQSGGCGGFGKGNFSELFKSIEEYEKTLEAKQLVG

SEQ ID NO: 144 - protein, 446 aa, Capsella rubella
MGHQNAAVPEIQLNNDGDAASSPGFKLVGFSNFVRKNPKSDKFKVKRFHHIEFWCGDATNVARRFSWGLGMRFTAKSDLSTGNMVHASYLLTSGDLRFLFTAPYSPSLSAGEIKLTATASIPSFDHVSSRSFFSSHGLGVRAVAIEVDDADSAFSISVANGAIPSSPPIVLDESVTIAEVKLYGDVVLRYVSYKGGDTGQPQFLPGFEPVEDTSSFPLDYGIRRLDHAVGNVPELGPALTYIAGFTGFHQFAEFTADDVGTAESGLNSAVLASNDEMVLLPINEPVHGTKRKSQIQTYLEHNEGAGLQHLALMSEDIFRTLREMRKRSSVGGFDFMPSPPPTYYQNLKKRVGDVLSDEQIKECEELGILVDRDDQGTLLQIFTKPLGDRPTIFIEIIQRIGCMMKDDEGKAYQSGGCGGFGKGNFSELFKSIEEYEKTLEAKQLVG

SEQ ID NO: 145 - protein, 432 aa, Chlamydomonas reinhardtii
MGAGGAGTGDREGGIKLVGYKNFVRQNPLSDKFTVHKFHHIDFWCGDATNTSKRFSYGLGMPLVAKSDQSTNNQLFASYVLRSNDLVFTFTAPYSRKCASVSEGVPLRHYNIDHAYEFINSHGLAVRAVGLLVDDAKTAYEVSVAHGAKGVLPPVELRDEASGTSQVISEVLLYGEVVLRYVSGSFQGPFLAGYTPVTDSAVTSFGLQRLDHAVGNTHDLIKAVEYITGFTGFHEFSEFVAEDVGTVDSGLNSMVLASNNEAVLLPVNEPTFGTPRKSQIQTYLEQNEGPGLQHLALLSNDIFTTLREMRARSELGGFEFMPRANAKYYKDMYARIGDSLTPQQYREVEELGILVDKDDQGVLLQIFTKPLGDRPTVFIEIIQRVGCMREVKEPATGAVVGTEQAAGCGGFGKGNFGALFKSIEDYERTLNV

SEQ ID NO: 146 - protein, 462 aa, Chlorella fusca
MVELVGYSNFKRHNPRSDKFGINKFHHIEFWCADATNTFKRYMGSNRRRFQHGLGMTLVAKSDHSTGNSKYCSYVLQSNDLVFTFTAPYSRKCFAESDKSVEPLPDFNQQQAFEFVCTHGFAARAIGLLVDDAKEAYVVSVAHGAKGVRPPTCLHDGAGGAAVVSEVIIYGDVVLRYISGKWQGPYLPGYAATPDEPHICYGLQRLDHAVGNVPKLIEQVEHVMRFTGFHEFAEFVAADVGTVDSGLNSMVLANNSEMVLLPINEPTFGTKRKSQIQTYLEQNEGPGLQHLALKTHNVIATMREMFSRSRCGGFEFQSAPGPSYYKRSREKIGDVLSADEWKAVEEMGILVDQDDQGVLMQIFTKPLSDRPTIFIEIIERRGCLKETQPSTMNGAAEQAVPGNESGTAAGKGQGLEERFRDIVAVKEGGIIVEQAAGCGGFGKGNFSELFKSIEEYEKTLQV

SEQ ID NO: 147 - protein, 452 aa, Cocos nucifera
MGTAAEEQLGKKDFKLVGFERFVRTNPRSDRFPVLSFHHVEFWTCDATNATGRFSFGLGMRLAARSDLSTGNPLHASFLLRSGSLQFLFSSPYSPFTPAAAAVAAVAAPVADPCRPSIPSFDPDLARRFTASHGLAVRAVAVRVADAAAAFHISVAHGARPAFTPTDLGAGFALSEVELYGDVVLRYVSLPDPGSSQNPSKIPSFLPGFEDVEESGSDSPDYGLRRLDHAVGNVPELAPVLAYIAGFMGFHEFAEFTAEDVGTAESGLNSMVLANNEEMVLLPLNEPVHGTKRRSQIQTYLDHNGGPGVQHLALASDDVLRTLREMKAKSAMGGFEFLAPPPPTYYQGVRRRAGNVLTEEQIKECQELGVLVDRDDQGVLLQIFTKPVGDRPTIFLEIIQRVGCMMKDEEGREYQKGGCGGFGKGNFSALFQSIEDYEKSLEAKQTTVVPVA

SEQ ID NO: 148 - protein, 442 aa, Daucus carota
MGKKQSEAEILSSNSSNTSPATFKLVGFNNFVRANPKSDHFAVKRFHHIEFWCGDATNTSRRFSWGLGMPLVAKSDLSTGNSVHASYLVRSANLSFVFTAPYSPSTTTSSGSAAIPSFSASGFHSFAAKHGLAVRAIALEVADVAAAFEASVARGARPASAPVELGDQAWLAEVELYGDVVLRFVSFGREEGLFLPGFEAVEGMASFPDLDYGIRRLDHAVGNVTELGPVVEYIKGFTGFHEFAEFTAEDVGTLESGLNSVVLANNEEMVLLPLNEPVYGTKRKSQIQTYLEHNEGAGVQHLALVSEDIFRTLREMRKRSCLGGFEFMPSPPPTYYKNLKNRVGDVLSDEQIKECEDLGILVDRDDQGTLLQIFTKPVGDRPTLFIEIIQRVGCMLKDDAGQMYQKGGCGGFGKGNFSELFKSIEEYEKTLEAKQITGSAAA

SEQ ID NO: 149 - protein, 439 aa, Dichanthelium oligosanthes
MPPTPTTPAAAAAAAPGAADQAAFRLVGHRSFVRVNPRSDRFHALAFHHVELWCADAASAAGRFSFGLGAPLAARSDLSTGNSAHASLLLRSGSLAFLFTAPYAQGVDAATATLPSFSAPAARRFAADHGLAVRAIAVRVADAEDAFRASVDAGARPAFEPVELGLGFRLAEVELYGDVVLRYVSYPDAPDAPFLPGFEDVSNPGARDYGLRRFDHIVGNVPELAPVASYIAGFTGFHEFAEFTAEDVGTAESGLNSVVLANNTENVLIPLNEPVHGTKRRSQIQTFLDHHGGPGVQHMALASDDVLRTLREMRARSAMGGFEFMAPPQPDYYDGVRRRAGDVLSEAQIKECQELGVLVDRDDQGVLLQIFTKPVGDRPTMFLEIIQRVGCMEKDEQGQEYQKGGCGGFGKGNFSQLFKSIEEYEKSLEAKQAVSAQGS

SEQ ID NO: 150 - protein, 443 aa, Digitaria exilis
MPPTPTPAAAAAATGGVAPAGAEQAAFRLVGNRNFVRVNPRSDRFGALAFHHVELWCADAASAAGRFSFGLGAPLAARSDLSTGNSAHASLLLRSGSLAFLFTAPYAAADAAAATASLPSFSAPAARRFAADHGLAVRAVALRVADAEDAFRASVDAGARPAFEPVELGLGFRLAEVELYGDVVLRYVSYPDAEDAPFLPGFENVSNPGALDYGLRRFDHIVGNVPELAPVAAYVAGFTGFHEFAEFTAEDVGTAESGLNSVVLANNAENVLLPLNEPVHGTKRRSQIQTYLDHHGGPGVQHMALASNDVLRTLREMQARSAMGGFEFLEPPPPNYYDGVRRRAGDVLTEEQIKECQELGVLVDRDDQGVLLQIFTKPVGDRPTLFLEIIQRIGCMEKDEQGQEYQKGGCGGFGKGNFSQLFKSIEEYEKSLEAKQAATVQGS

SEQ ID NO: 151 - protein, 440 aa, Eragrostis curvula
MPPTPTTITGGAAAPPPAPADQAAFRLVGHRRFVRANPRTDRFHALAFHHVELWCTDAASAAGRFSFGLGAPLAARSDLSTGNSAHASLLLRSGSLAFLFTAPYARGVDAATASLPSFSADAARRFAADHGLAVRAVALRVADAEDAFRASVAAGARPAFEPVELGLGFRLAEVELYGDVVLRYVSYPDDEDSPFLPGFEDVSNPGALDYGLRRFDHIVGNVPELAPVAAYVAGFTGFHEFAEFTAEDVGTAESGLNSVVLANNAETVLLPLNEPVHGTKRRSQIQTYLDHHGGPGVQHIALSSNDVLRTLREMRARSAMGGFEFLAPPPPTYYDGVRRRAGDVLSEAQIKECQELGVLVDRDDQGVLLQIFTKPVGDRPTLFLEIIQRIGCMEKDERGQEYQKGGCGGFGKGNFSELFKSIEEYEKSLEAKQAGAVQQF

SEQ ID NO: 152 - protein, 445 aa, Eutrema sallsugineum
MGHQNTAVSESQQHDGAAASESPGFKLVGFSKFVRKNPKSDKFKVKRFHHIEFWCGDATNVARRFSWGLGMRFSAKSDLSTGNMVHASYLLTSGDLRFLFTAAYSPSLAAGEIRPTATASIPSFDHISWRSFFSSHGLGVRAVAIEVEDAESAFSISVANGAIPSSPPIVLDEAVTIAEVKLYGDVVLRYVSHNGVNTDKSDFLPGFELVEDTSSFPLDYGIRRLDHAVGNVPELGPALTYVAGFTGFHQFAEFTADDVGTADSGLNSAVLANNDEMVLLPINEPVHGTKRKSQIQTYLEHNEGAGLQHLALMSEDIFRTLREMRKRSSVGGFDFMPAPPPTYYQNLKKRVGDVLSDEQIKECEELGILVDRDDQGTLLQIFTKPLGDRPTIFIEIIQRIGCMKKDEEGKVYQSGGCGGFGKGNFSELFKSIEEYEKTLEAKQLV

SEQ ID NO: 153 - protein, 490 aa, Glycine max
MPMYTPSLSAPSSNHIQPSVTLPLYITTTKLNLKQQHHTTPMPIPMCNEIQAQAQAQAQAQPGFKLVGFKNFVRTNPKSDRFQVNRFHHIEFWCTDATNASRRFSWGLGMPIVAKSDLSTGNQIHASYLLRSGDLSFLFSAPYSPSLSAGSSAASSASIPSFDAATCLAFAAKHGFGVRAIALEVADAEAAFSASVAKGAEPASPPVLVDDRTGFAEVRLYGDVVLRYVSYKDAAPQAAHADPSRWFLPGFEAAASSSSFPELDYGIRRLDHAVGNVPELAPAVRYLKGFSGFHEFAEFTAEDVGTSESGLNSVVLANNSETVLLPLNEPVYGTKRKSQIETYLEHNEGAGVQHLALVTHDIFTTLREMRKRSFLGGFEFMPSPPPTYYANLHNRAADVLTVDQIKQCEELGILVDRDDQGTLLQIFTKPVGDRPTIFIEIIQRIGCMVEDEEGKVYQKGACGGFGKGNFSELFKSIEEYEKTLEAKRTA

SEQ ID NO: 154 - protein, 436 aa, Gossypium hirsutum
MVQTNQSGSGNDFKLVGFSNFVRSNPKSDRFTVKRFHHIEFWCTDATNVARRFSWGLGMQFVAKSDLSTGNLTHASYLLRSGDLNFLFTAPYSPSIAVAQNLSPQSTASIPSFDHSLCRSFAATHGLGVRAIAIEVDDAETAFTTSVTHGALPFSPPTPLGDVATIAEVKLYGDVVLRYVSYTTTINSDHDFSPGFEKIEDALSYPLDYGLRRLDHAVGNVPELGPAVSYVKSFTGFHEFAEFTAEDVGTSESGLNSVVLANNEEMVLLPMNEPVFGTKRKSQIQTYLEHNEGAGVQHLALVSEDIFKTLREMRKRSFVGGFEFMPSPPPTYYKKLKQRAGDILSDEQIKECEELGILVDRDDQGTLLQIFTKPVGDRPTIFIEIIQRIGCLVKDEEGKQYQKGGCGGFGKGNFSELFKSIEEYEKSLEAKQSQNP

SEQ ID NO: 155 - protein, 451 aa, Helianthus annuus
MGTEATVAAVVAGDESDHPTSAFKLVGFKNFIRTNPMSDKFTVKNFHHIEFWCSDATNTARRFSWGLGMPIIFKSDLSTGNSTHASYLLRSGHLNFLFTAPYSPSISPTTTTASIPTFSHSASRHFTATHGLAVRAIAVEVEDAETAFAVSVANGAKPSSPPVTLGHNDVVLSEVKLYGDVVLRYVSYKNNTNNNNNNDNDNYIFLPGFEAMDKTSSFQELDYGIRRLDHAVGNVPELAPAVDYVKSFTGFHEFAEFTAEDVGTSESGLNSVVLACNSEMVLIPMNEPVYGTKRKSQIQTYLEHNEGAGVQHLALASEDIFRTLREMRKRSGVGGFEFMPSPPPTYYRNLKSRAGDVLSDEQIKECEELGILVDRDDQGTLLQIFTKPVGDRPTIFIEIIQRVGCMVKDDEGKVQQKAGCGGFGKGNFSELFKSIEEYEKTLEARSTTAAA

SEQ ID NO: 156 - protein, 434 aa, Hordeum vulgare
MPPTPTTPAATGAAAAVTPEHARPHRMVRFNPRSDRFHTLSFHHVEFWCADAASAAGRFAFALGAPLAARSDLSTGNSAHASQLLRSGSLAFLFTAPYANGCDAATASLPSFSADAARRFSADHGIAVRSVALRVADAAEAFRASVDGGARPAFAPVDLGRGFGFAEVELYGDVVLRFVSHPDGTDVPFLPGFEGVTNPDAVDYGLTRFDHVVGNVPELAPAAAYIAGFTGFHEFAEFTAEDVGTTESGLNSVVLANNSEGVLLPLNEPVHGTKRRSQIQTFLEHHGGPGVQHIAVASSDVLRTLRKMRARSAMGGFDFLPPPLPKYYEGVRRLAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 157 - protein, 456 aa, Ipomea triloba
MGKETSSSGNDHSTAENGGFKLVGFKDFVRVNPKSDHFAVKRFHHVEFWCGDATNTARRFSWALGMPIAAKSDLSTGNSAHASYLLRACGGELQFLFTAPYASSICNSASAAIPSFSADAHRAFAATHGLAVRAVALVVEDTRAAFSASVSRGAKPVSEPVTLNNQVVLAEVHLYGDVVLRFVSYLSDASAVTFLPGFEAMDGTASFQDLDYGIRRLDHAVGNVHELAPAVDYLKSFTGFHEFAEFTAEDVGTAESGLNSVVLANNDETVLLPLNEPVYGTKRKSQIQTYLEHNEGPGLQHLALTSEDIFRTLREMRKRSGIGGFEFMPSPPPTYYKNLKSRAGDVLNDEQIKECEELGILVDRDDQGTLLQIFTKPVGDRPTMFIEIIQRVGCMMKKEDGQLYQKGGCGGFGKGNFSELFKSIEEYEKMLEAKQATRAGEVSSSTMIYECDSVIH

SEQ ID NO: 158 - protein, 449 aa, Lactuca salinga
MGTEAKLTNGNTVTGEQTGSAFKLVGFNNFIRTNPMSDKFSVKRFHHIEFWCSDATNTARRFSWGLGMPIVQKSDLSTGNATHASYLLRSGQLNFLFTAPYSPSISSAATSTTASIPSFSHTDCRNFTASHGLAVRSVAIEVEDAEIAFSVSVSHGAKPSSSPITLGNNDVVLSEVKLYGDVVLRYISFKNPNFDAISNFLPGFEPVEKTSSFPDLDYGIRRLDHAVGNVPELAPAVDYVKSFTGFHEFAEFTAEDVGTSESGLNSVVLACNSEMVLIPMNEPVYGTKRKSQIQTYLEHNEGAGVQHLALASEDIFRTLREMRKRSGIGGFEFMPSPPPTYYRNLKNRVGDVLTDEQIKECEELGILVDRDDQGTLLQIFTKPVGDRPTIFIEIIQRVGCMLKDDEGKVQQKAGCGGFGKGNFSELFKSIEEYEKTLEARTTSEPTAAA

SEQ ID NO: 159 - protein, 443 aa, Lolium rigidum
MPPTPTTATGAAAGVPAVTPEHAPRRFPRVVRVNPRSDRFPVLAFHHVELWCADAASAAGRFAFALGAPLAARSDLSTGNSAHASLLLRSGALAFLFTAPYAPPPEESAAASASASIPSFSAPAARAFSAAHGLAVRSIALRVADAAEAFRASVAGGARPAFPPADLTHGYTLAEVELYGDVVLRFISYPDNNNDTNLPFLPGFERVSSPGAVDYGLTRFDHVVGNVPEMAPVLAYMKGFLGFHEFAEFTAEDVGTAESGLNSVVLANNSEAVLLPLNEPVHGTKRRSQIQTYLDYHGGPGVQHIALASRDVLRTLREMRARTPMGGFEFMAPPQAKYYDGVRRIAGDVLSEEQIKECQELGVLVDRDDQGVLLQIFTKPVGDRPTFFLEMIQRIGCMEKDEVGQEYQKGGCGGFGKGNFSELFKSIEDYEKTLEAKQSVVAQ

SEQ ID NO: 160 - protein, 457 aa, Malus domestica
MTASHSPPATATAIRSTMGQENDTHFNLVGFSNFVRTNPRSDRFRVHRFHHVEFWCTDATNAALRFSWGLGMPMVAKSDLSTGNQTHASYLLRSGDLNFLFTAPYSPTLTRTADPARPSNSSASIPTFDHTASRAFSATHGLGVRAVAIQVEDAVSAFTTSVAHGAKPSAAPILLDNRVTIAEVQLYGDVVLRYVSYSDHNHLTDPDPNLWFLPGFEPLPSSFPIDFGIRRLDHAVGNVEDLKSVISYVKGFTGFHEFAEFTADDVGTSESGLNSVVLANNEEMVLLPMNEPVYGTKRKSQIQTYLEHNEGAGLQHLALVSEDIFRTLREMRSRSSVGGFQFMPKPPPTYYENLKKRAGDVLTDEQIKECEELGILVDRDDQGTLLQIFTKPVGDRPTMFIEIIQRVGCMLKNEEGKVHQKGGCGGFGKGNFSELFKSIEEYEKTLEAKQIAEPAAA

SEQ ID NO: 161 - protein, 443 aa, Microthlaspi erraticum
MGHENDAASESHQHDGAAASGFKLVGFSKFVRKNPKSDRFKVKRFHHIEFWCGDATNVARRFSWGLGMRFSAKSDLSTGNMVHASYLLTSGDLRFLFTAPYSPSLAAGDTRLTATASIPSFDHVSCRSFISSHGLGVRAVAIEVEDAESAFSISVANGAIPSSPPFVLDEAVTIAEVKLYGDVVLRYVSYKGVDSDKSDFLPGFEPVEDTSSFPLDYGIRRLDHAVGNVPELGPALTYVAGFTGFHQFAEFTADDVGTAESGLNSAVLASNDEMVLLPMNEPVHGTKRKSQIQTFLEHNEGAGLQHLALMSEDIFRTLREMRKRSSVGGFDFMPSPPPTYYQNLKKRVGDVLSDEQIKECEELGILVDRDDQGTLLQIFTKPLGDRPTIFIEIIQRIGCMKKDEEGKVHQSGGCGGFGKGNFSELFKSIEEYEKTLEAKPLVG

SEQ ID NO: 162 - protein, 435 aa, Miscanthus lutarioriparius
MPPTPTAATTGAVVAAASAEQAAFRLVGHRNFVRVNPRSDRFHTFAFHHVELWCADAASAAGRFSFGLGAPLAARSDLSTGNTAHASLLLRSGALAFLFTAPYAHGADAATASLPSFSAADARRFAVDHGLAVRAVALRVADAEDAFRASVAAGARPAFEPVELGLGFRLAEVELYGDVVLRYVSYPDDADAPFLPGFEGVSSPGAVDYGLRRFDHIVGNVPELAPAAAYFAGFTGFHEFAEFTAEDVGTTESGLNSVVLANNAENVLLPLNEPVHGTKRRSQIQTYLDHHGGPGVQHMALASDDVLRTLREMQARSAMGGFEFMAPPPPDYYDGVRRRARDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAV

SEQ ID NO: 163 - protein, 445 aa, Musa balbisiana
MGKEATENFKLVGFTRFVWANTRSDRFPVLGFHHVEFWAADATNSAGRFSFGLGMPLAARSDLSTGNPFFASYLLRSASLRFLFSAPYGGPAAADAAANSATPSPIPSFDADRARRFSVDHGLAVRAIAIEVADAAEAFRISVAHGARPSFPPADLGEGFALAEVELYGDVVLRYVSRPKPPPFPSEAHQTPIKVPAFLPGFEDIDESETCSLTYGIRRLDHAVGNVPELAPAVRYIAGFTGFHEFAEFTADDVGTAESGLNSVVLANNEETVLVPLNEPVHGTKRRSQIQTYLDHNGGAGVQHLALASDDVLKTLREMRARSAMGGFDFMPPPPPNYYQGVRRRAGDVLTEEQIKECQELGVLVDRDDQGVLLQIFTKPVGDRPTIFIEIIQRIGCMVKDKEGKEYQKGGCGGFGKGNFSELFKSIEEYEKSLEAKQATTVPSA

SEQ ID NO: 164 - protein, 447 aa, Nicotiana tabacum
MGKLETVTTTSAAAADDSPELTTNFKLVGFKNFIRINPRSDFFSVKRFHHIEFWCGDATNTVRRFSWSLGMPIAAKSDLSTGNSVHASYLLRPVSGSLQFLFTAPYSPSISTPSSAAIPSFSTSVHRSFTATHGLGVRAVALEVENAYTAFSASVSRGAKPMFEPVTIDERVAVAEVHLYGDVVLRFVSYLKDADSLVFLPGFEAMDETASYKELDYGIRRLDHAVGNVPELGPVVDYIKRFTGFHEFAEFTAEDVGTAESGLNSMVLANNDETVLLPLNEPVYGTKRKSQIQTYLEHNEGAGVQHLALVTEDIFKTLTEMRKRSGVGGFEFMPSPPPTYYKNLKNRAGDVLTDEQIQACEELGILVDRDDQGTLLQIFTKPVGDRPTIFIEIIQRIGCMLKDEKGQVYQKGGCGGFGKGNFSELFRSIEEYEKTLEAKQVNQVAAA

SEQ ID NO: 165 - protein, 441 aa, Oryza brachyantha
MPPTPTATATGGAVSAADNAGFRLVGHRRFVRANPRSDRFHALAFHHVELWCADAASAAGRFSFALGAPLAARSDLSTGNSAHASLLLRSGSVAFLFTAPYAHRAGGEGGATASASLPSFSADAARGFAADHGLAVRAVALRVADAAEAFRASVAAGARPAFEPADLGAGFGLAEVELYGDVVLRFVSYPDGAASFLPGFEDVSNPGTQDYGLRRFDHVVGNVPELAPVAAYIANFTGFHDFAEFTAEDVGTAESGLNSVVLANNAETVLLPLNEPVHGTKRRSQIQTYLDHHGGPGVQHIALASDDVLRTLREMRARSAMGGFEFLAPPPPNYYDGVRRRAGDVLSEEQIKECQELGVLVDRDDQGVLLQIFTKPIGDRPTLFLEMIQRIGCMEKDESGQEHQKGGCGGFGKGNFSELFKSIEEYEKSLEAKQASSVQGS

SEQ ID NO: 166 - protein, 438 aa, Oryza meyeriana
MPPTPPATGGAVSAAAAAEHAGFRLVGHRRFVRANPRTDRFHALAFHHVELWCADAASATGRFSFALGAPLAARSDLSTGNSAHASLLLRSGSVAFLFTAPYARGVDESTASIPSFSANAARRFAAEHGLAVRAVALRVADAADAFRASVAAGARPAFEPADLGGGFGLAEVELYGDVVLRYVSYPDGAAASFLPGFEDVRNPGAMDYGLRRFDHVVGNVPELAPVAAYIAGFTGFHEFAEFTAEDVGTAESGLNSVVLANNTETVLLPLNEPVHGTKRRSQIQTYLDHHGGPGVQHIALASDDVLRTLREMRARSAMGGFEFLAPPPPNYYDSVRRRAGDVLSEEQIKECQELGVLVDRDDQGVLLQIFTKPVGDRPTFFLEMIQRIGCMEKDESGQEYQKGGCGGFGKGNFSELFKSIEEYEKSLEAKQAATVQGS

SEQ ID NO: 167 - protein, 446 aa, Oryza sativa japonica
MPPTPTPTATTGAVSAAAAAGENAGFRLVGHRRFVRANPRSDRFQALAFHHVELWCADAASAAGRFAFALGAPLAARSDLSTGNSAHASLLLRSASVAFLFTAPYGGDHGVGADAATTASIPSFSPGAARRFAADHGLAVHAVALRVADAADAFRASVAAGARPAFQPADLGGGFGLAEVELYGDVVLRFVSHPDGADAPFLPGFEGVSNPGAVDYGLRRFDHVVGNVPELAPVAAYISGFTGFHEFAEFTAEDVGTAESGLNSVVLANNAETVLLPLNEPVHGTKRRSQIQTYLDHHGGPGVQHIALASDDVLGTLREMRARSAMGGFEFLAPPPPNYYDGVRRRAGDVLSEEQINECQELGVLVDRDDQGVLLQIFTKPVGDRPTFFLEMIQRIGCMEKDESGQEYQKGGCGGFGKGNFSELFKSIEEYEKSLEAKQAPTVQGS

SEQ ID NO: 168 - protein, 439 aa, Panicum hallii
MPPTPTPAAAAAPGAAVAPEQAAFRLVGHRGFVRVNPRSDRFHTLAFHHVELWCADAASAAGRFSFGLGAPLAARSDLSTGNSAHASLLLRSGSLAFLFTAPYARGADPATAALPSFSAPAARRFAADHGLAVRAIALRVADAEDAFRSSVAAGARPAFEPVDLGLGFRLAEVELYGDVVLRYVSYPDAADAPFLPGFEDVSNPGAVDYGLRRFDHVVGNVPELAPVAAYVAGFTGFHEFAEFTAEDVGTAESGLNSMVLANNSENVLIPLNEPVHGTKRRSQIQTFLDHHGGPGVQHIAVASNDVLRTLREMQARSAMGGFEFMAPPPPNYYDGVRRRAGDVLSEEQIKECQELGVLVDRDDQGVLLQIFTKPVGDRPTFFLEIIQRIGCMEKDEQGQEYQKGGCGGFGKGNFSQLFKSIEDYEKSLEAKQAAAVQGS

SEQ ID NO: 169 - protein, 439 aa, Panicum milliaceum
MPPTPTPAAAAAPGAAVAPEQAAFRLVGHRGFVRVNPRSDRFHTLAFHHVELWCADAASAAGRFSFGLGAPLAARSDLSTGNSAHASLLLRSGSLAFLFTAPYARGADPATAALPSFSAPAARRFAADHGLAVRAIALRVADAEDAFRASVAAGARPAFEPVELGLGFRLAEVELYGDVVLRYVSYPDAADAPFLPGFEDVSNPGAVDYGLRRFDHVVGNVPELAPVAAYVAGFTGFHEFAEFTAEDVGTAESGLNSMVLANNSENVLIPLNEPVHGTKRRSQIQTFLDHHGGPGVQHIAVASNDVLRTLREMQARSAMGGFEFMAPPPPNYYDGVRRRAGDVLSEEQIKECQELGVLVDRDDQGVLLQIFTKPVGDRPTFFLEIIQRIGCMEKDEQGQEYQKGGCGGFGKGNFSQLFKSIEDYEKSLEAKQAAAVQGS

SEQ ID NO: 170 - protein, 439 aa, Panicum virgatum
MPPTPTPAAAAAPGAAAAPEEASFRLVGHRSFVRVNPRSDRFHALAFHHVELWCADAASAAGRFSFGLGAPLAARSDLSTGNSAHASLLLRSGSLAFLFTAPYARGADPATATLPSFSAPAARRFAADHGLAVRAVALRVADAEDAFHASVAAGARPAFEPVELGLGFRLAEVELYGDVVLRYVSYPDAAGAPFLPGFEDVSNPGAVDYGLRRFDHIVGNVPELAPVAAYISGFTGFHEFAEFTAEDVGTSESGLNSVVLANNSENVLIPLNEPVHGTKRRSQIQTFLDHHGGPGVQHIAVASNDVLRTLREMQARSAMGGFEFMAPPPPNYYDGVRRRAGDVLSEEQIKECQELGVLVDRDDQGVLLQIFTKPVGDRPTFFLEIIQRIGCMEKDEQGQEYQKGGCGGFGKGNFSQLFKSIEDYEKSLEAKQAAAVQGS

SEQ ID NO: 171 - protein, 451 aa, Phoenix dactylifera
MGTKAEIASEEEVGKKDFKLVGFKRFVRTNPRTDRFPVLGFHHIEFWTADATNAAGRFSFGLGMRLAARSDLSTGNPFHASFLLRSGSLQFLFSSPYSPFTPAAAGAPAGDPGSPSIPSFDPDRARRFTGSHGLAVRAVAVRVADAAAAFHTSVAHGARPALPPADLGAGFALSEVELYGDVVLRYVSLPAPESSQNPSKIPSFLPGFEDVEESGPHSPDYGLRRLDHAVGNVPEMAPVLAYIAGFTGFHEFAEFTAEDVGTAESGLNSMVLANNEETVLLPVNEPVHGTKRRSQIQTYLDHNGGAGLQHLALASDDVLRTLREMKAKSAMGGFEFLAPPPPNYYQGVRRRAGDVLTEEQIKECQELGVLVDRDDQGVLLQIFTKPIGDRPTIFLEIIQRIGCMMKDEEGREYQKGGCGGFGKGNFAELFKSIEEYEKSLEAKQTTVVPVT

SEQ ID NO: 172 - protein, 441 aa, Raphanus raphanistrum
MGHENAVVSESQHHDDAASPGFKLVGFSKFVRKNPKSDKFKVKRFHHIEFWCGDATNVARRFSWGLGMRLSAKSDLSTGNMVHASYLLTSGDLRFLFTAPYSPSLSSGEARPNATASIPSFDHVSCRSFFSSHGLGVRAVAIEVEDAESAFSISVANGAIPSSPPNVLNGAVTIAEVKLYGDVVLRYVSYKEGDAVTFLPGFELVDDTSSFPLDYGIRRLDHAVGNVPELGPALTYLAGFTGFHQFAEFTADDVGTAESGLNSAVLANNDEMVLLPINEPVHGTKRKSQIQTYLEHNEGAGLQHLALMSEDIFRTLREMRKRSGVGGFDFMPSPPPTYYKNLKKRVGDVLSEEQIKECEELGILVDRDDQGTLLQIFTKPLGDRPTIFIEIIQRVGCMKKDEEGKVYQSGGCGGFGKGNFSELFKSIEEYEKTLEAKQLVG

SEQ ID NO: 173 - protein, 441 aa, Ricinus communis
MGKENDVVSSTPEQGFKLVGFSNFVRANPKSDRFKVKRFHHVEFWCTDATNTARRFSWGLGMPIVAKSDLSTGNVTHASYLLRSGDLNFLFTAPYSPSIAAMENLSHTATASIPTFSHETCRYFSAKHGLAVRAIAIEVEDAEIAFRTSASHGAKPSAEPILLDNRAVIAEVHLYGDVVLRYISYKNEGSNFEFLPRFEPVDEATSFPVDYGIRRLDHAVGNVPELAPVVSYVKQFTGFHEFAEFTTEDVGTSESGLNSVVLANNEETVLIPMNEPVFGTKRKSQIQTYLEHNEGAGVQHLALASEDIFRTLREMRKRSGVGGFEFMPSPPPTYYKNLKNRAGDVLSDEQIKECEELGILVDRDDQGTLLQIFTKPVGDRPTIFIEIIQRVGCMLKDDAGKEYQKGGCGGFGKGNFSELFKSIEEFEKTLEARRVTEAASA

SEQ ID NO: 174 - protein, 425 aa, Setaria italica
  misc_feature (221)..(221): Xaa can be any naturally occurring amino acid
MAPTPAAAATVGHRSFVRANPRSDRFHALSFHHVELWCADAASAAGRFSFALGAPLAARSDHSTGNSAHASLLLRSDSLAFLFTAPYAHGVDAATASLPSFSAPAARRFAADHGLAVRAIALRVADAEDSFRASVAAGARPAFEPVELGLGFRLAEVELYGDVVLRYVSHPDATDDAPFLPGFEDVISSRGAPDYGLRRFDHVVGNVPELAPVAAYVAGFXGFHEFAEFTAEDVGTAESGLNAVVLANNSETVLLPLTEPVHGTKRRSQIQTYLDHHGGPGVQHIAMASDDVLRTLRKMRARSAMGGFEFLAPPPPNYYDGVRRRAGDVLSEAQIKECQELGVLVDRDDQGVLLQIFTKPVGDRPTFFLEIIQRIGCMEKDETGLDYQRGACGGFGKGNFSELFKSVEEYEKSLEAKQADALQES

SEQ ID NO: 175 - protein, 424 aa, Setaria viridis
MAPTPPAAAATVGHRSFVRANPRSDRFHALSFHHVELWCADAASAAGRFSFALGAPLAARSDHSTGNSAHASLLLRSDSLAFLFTAPYAHGVDAATASLPSFSAPSARRFAGDHGLAVRAIGLRVADAEDAFRASVAAGARPAFEPVELGLGFRLAEVELYGDVVLRYVSYPDAEDAPFLPGFENVNNPGALDYGLRRFDHIVGSLPELAPVAAYVAGFTGFHEFAEFTAEDVGTAESGLNAVVLANNSETVLLPLTEPVHGTKRRSQIQTYLDHHGGPGVQHIAMASDDVLRTLRKMRARSAMGGFEFLAPPPPNYYDGVRRRAGDVLSEAQIKECQELGVLVDRDDQGVLLQIFTKPVGDRPTFFLEIIQRIGCMEKDETGLDYQRGACGGFGKGNFSELFKSLEEYEKSLEAKQADALQGS

SEQ ID NO: 176 - protein, 443 aa, Sinapis alba
MGHQNAVVSETQQHDDVAASASPGFKLVGFSKFVRKNPKSDKFKVKRFHHIEFWCGDATNVARRFSWGLGMRFSAKSDLSTGNMVHASYLLTSGDLRFLFTAPYSPSHSAGETPQTATASIPSFDHVSCSSFFSSHGLGVRAVAIEVEDAESAFSISVANGAIPSSPPNVLDGTVTIAEVKLYGDVVLRYVSYKGGDINFLPRFEPVEDTSSFPLDYGIRRLDHAVGNVPELGPALTYLAGFTGFHQFAEFTADDVGTAESGLNSAVLANNDEMVLLPINEPVHGTKRKSQIQTYLEHNEGAGLQHLALMSEDIFKTLREMRKRSSVGGFDFMPSPPPTYYKNLKNRVGDVLSDEQIKECEELGILVDRDDQGTLLQIFTKPLGDRPTIFIEIIQRVGCMKKDEEGKVYQSGGCGGFGKGNFSELFKSIEEYEKTLEAKQLVS

SEQ ID NO: 177 - protein, 440 aa, Sorghum bicolor
MPPTPTTAAATGAAVAAASAEQAAFRLVGHRNFVRVNPRSDRFHTLAFHHVELWCADAASAAGRFSFGLGAPLAARSDLSTGNTAHASLLLRSGALAFLFTAPYAHGADAATASLPSFSAAEARRFAADHGLAVRAVALRVADAEDAFRASVAAGARPAFEPVELGLGFRLAEVELYGDVVLRYVSYPDDADASFLPGFVGVTSPGAADYGLRRFDHIVGNVPELAPAAAYFAGFTGFHEFAEFTAEDVGTTESGLNSMVLANNAENVLLPLNEPVHGTKRRSQIQTYLDHHGGPGVQHMALASDDVLRTLREMQARSAMGGFEFMAPPAPEYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAQGS

SEQ ID NO: 178 - protein, 444 aa, Sorghum halapense
MPPTPTTAAATGAAVAAASAEQAAFRLVGHRNFLRVNPRSDRFHTLAFHHVELWCADAASAAGRFSFGLGAPLAARSDLSTGNTAHASLLLRSGALAFLFTAPYAHGADAATASLPSFSAAEAAAEARRFAADHGLAVRAVALRVADAEDAFRASVAAGARPAFEPVELGLGFRLAEVELYGDVVLRYVSYPDDADASFLPGFVGVTSPGAADYGLRRFDHIVGNVPELAPAAAYFAGFTGFHEFAEFTAEDVGTSESGLNSMVLANNAENVLLPLNEPVHGTKRRSQIQTFLDHHGGPGVQHMALASDDVLRTLREMQARSAMGGFEFMAPPAPEYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAQGS

SEQ ID NO: 179 - protein, 362 aa, Synechococcus sp. PCC 7336
MHDFCPMLGFDHLEFYVGNARQAALYYSTGFGFVNTAYRGLETGDRKVSSYVMEQGKIRFVLTTALSSDHPIARSVLAHGDGIAVMALAVPDAERAYQQAIGRGAVGAIPPTAESDAGGTLHYAAIRAYGDVVLKFVDRTQYRGVFAPHFQPRQRSNNNGVGFTHIDHVVGNVEVGAMDRWAQFLADSLGFELLVHFDEQTISTEYSALRSKVMQDRSGRIKLPINEPAAGLHKSQIQEYLDYNNGPGIQHVACATGDIIATVSHLRNSGIKFLDIPPAYYEDLQARVGPIDEPIEKLAELGILADRDKGGYLLQIFTQPVEDRPTLFFEAIERHGAQGFGEGNFKALFEAIEREQARRGNL

SEQ ID NO: 180 - protein, 439 aa, Synechocystis sp. PCC 7509
MGKETLSNSSQEFKLVGFSKFVRTNPKSDRFNVKRFHHIEFWCSDATNTARRFSWGLGMQLLAKSDLSTGNLTHASYLLRSGDLHFLFTAPYSPSIAHSQSLSPLSTASIPTFDHSACRSFSSSHGLAVRAIAIEVEDAETAFTTSVSHGALPSSPPVVLGDNVTLSEVKLYGDVVLRYISHNKNNDHDFFFLPGFEKIEDNLSYPLDYGIRRLDHAVGNVPELGPAVSYVKSFTGFHEFAEFTAEDVGTTESGLNSLVLANNDEMVLLPMNEPVFGTKRKSQIQTYLEHNEGAGVQHLALVSEDIFKTLREMRKRSLVGGFEFMPSPPPTYYKNLKTRAGDVLSDEQIKDCEELGILVDRDDQGTLLQIFTKPVGDRPTIFIEIIQRVGCMLKDEQGKQYQKGGCGGFGKGNFSELFKSIEEYEKTLGAKQIAESATA

SEQ ID NO: 181 - protein, 439 aa, Theobroma cacao; identical to SEQ ID NO: 180

SEQ ID NO: 182 - protein, 436 aa, Triticum aestivum
MPPTPTTPAATGAGAAAAVTPEHARPRRMVRFNPRSDRFHTLSFHHVEFWCADAASAAGRFAFALGAPLAARSDLSTGNSVHASQLLRSGNLAFLFTAPYANGCDAATASLPSFSADAARRFSADHGLAVRSIALRVADAAEAFRASVDGGARPAFSPVDLGRGFGFAEVELYGDVVLRFVSHPDGTDVPFLPGFEGVSNPGAVDYGLTRFDHVVGNVPELASAAAYVAGFTGFHEFAEFTTEDVGTAESGLNSMVLANNSEGVLLPLNEPVHGTKRRSQIQTFLEHHGGPGVQHIAVASSDVLRTLREMRARSAMGGFDFLPPPLPKYYEGVRRIAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 183 - protein, 436 aa, Triticum dicoccoides
MPPTPTTPAATGAAAAAAVTPEHARPRRMVRFNPRSDRFHTLAFHHVEFWCADAASAAGRFAFALGAPLAARSDLSTGNSVHASQLLRSGNLAFLFTAPYANGCDAATASLPSFSADAARRFSADHGLAVRSIALRVADAAEAFRASVDGGARPAFSPVDLGRGFGFAEVELYGDVVLRFVSHPDDTDVPFLPGFEGVSNPDAVDYGLTRFDHVVGNVPELAPAAAYVAGFAGFHEFAEFTTEDVGTAESGLNSMVLANNSEGVLLPLNEPVHGTKRRSQIQTFLEHHGGPGVQHIAVASSDVLRTLREMRARSAMGGFDFLPPPLPKYYEGVRRIAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQAS

SEQ ID NO: 184 - protein, 433 aa, Triticum turgidum
MPPTPTTPAATGAAAVTPEHARPRRMVRFNPRSDRFHTLAFHHVEFWCADAASAGGRFAFALGAPLAARSDLSTGNSVHASQLLRSGNLAFLFTAPYANGCDAATASLPSFSADAARQFSADHGLAVRSIALRVADAAEAFRASVDGGARPAFSPVDLGRGFGFAEVELYGDVVLRFVSHPDGTDVPFLPGFEGVSNPDAVDYGLTRFDHVVGNVPELAPAAAYVAGFTGFHEFAEFTTEDVGTAESGLNSMVLANNSEGVLLPLNEPVHGTKRRSQIQTFLEHHGGSGVQHIAVASSDVLRTLREMRARSAMGGFDFLPPPLPKYYEGVRRIAGDVLSEAQIKECQELGVLVDRDDQGVLLQIFTKPVGDRPTLFLEMIQRIGCMEKDERGEEYQKGGCGRLGKGNFSELFKSIEDYEKSLEAKQSAAVQGS

SEQ ID NO: 185 - protein, 433 aa, Vigna ungulatata
MGNQTQLQSDFKLVGFKNFVRTNPKSDRFQVNRFHHIEFWCTDATNAASRFSWGLGMPIVAKSDLSTGNQIHASYLLRSGDLSFLFSAPYSPSISAGGAATAAIPTFDAAACLSFAAKHGLGVRAIALEVADAEAAFRASVAHGAEPVSSPALLGGRTGFAEVRLYGDVVLRYVSYKDASHTSDTDPSRWFLPGFEASVAAFQGLDYGIRRLDHAVGNVPELAPAVSYLKNFTGFHEFAEFTAEDVGTSESGLNSVVLANNSETVLLPLNEPVYGTKRRSQIETYLEHNEGAGVQHLALVSNDIFRTLREMRKRSFVGGFEFMPSPPPTYYANLHKRAGDVLTVDQIKQCEELGILVDRDDQGTLLQIFTKPVGDRPTIFIEIIERVGCMVEDEEGKVYQKGACGGFGKGNFSELFKSIEEYEKTLLEARTKS

SEQ ID NO: 186 - protein, 444 aa, Zea mays
MPPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEEAFRTSVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAAAAQGS

SEQ ID NO: 187 - protein, 449 aa, Zingiber officinale
MGKEQATETAALEKSAAGLKLVGFTNFVRANPRSDRFPVLGFHHIEFWAADAANVAGRFSFSLGMPLSAKSDLSTGNPFFASYLLRSASLRFLVSAPYGGPAATDAAANFTGRSPIPSFDAAVARKFSIDHGLAVRAIAVEVADAAEAFRVSVARGARPAFPPADLGEGFALAEVEIYGDVVLRYISFPKVAPSSPKLPAFLPGFEDIEEFEATPPPTYGLQRIDHAVGNVPELLSAVRYIAGFTGFHEFAEFTSEDVGTAESGLNSVVLANNDETVLVPLNEPVHGTKRRSQIQTYLDHNGGAGVQHLALASYDILKTLREMRARTAMGGFDFMPSPPPNYYQGVRRRAGDVLTEEQIKECQELGVLVDRDDQGVLLQIFTKPIGDRPTIFIEIIQRIGCMVKDKEGREYQKGGCGGFGKGNFSELFKSIEEYEKSLEAKQAVAVPSA

SEQ ID NO: 188 - protein, 438 aa, Zizania palustri
MSPTHTHTGGAVAPAAAAEHAGFRLVGHRRFVRANPRSDRFHALAFHHVELWCADAASAAGRFSFALGAPLAARSDLSTGNSAHASLLLRSGSVAFLFTAPYARGVDAKTASLPSFSADAARIFSAEHGLAVRAVALRVADAADAFRASVAAGARPAFGPAELGGGFGFSEVELYGDVVLRFVSYPDSTDASFLPGFEDVSNPDAMDYGLRRFDHIVGNVPELAPVAAYIAGFTGFHEFAEFTAEDVGTAESGLNSVVLANNTETVLLPLNEPVHGTKRRSQIQTYLDHHGGPGVQHIALSSNDVLRTLRKMRARSAMGGFEFLAPPPPNYYDGVRRRAGDVLSEEQIKECQELGVLVDRDDQGVLLQIFTKPVGDRPTFFLEMIQRIGCMEKDESGQEYQKGGCGGFGKGNFSELFKSIEDYEKSLEAKQAVTVQGS

(19) United States
(12) Patent Application Publication
(10) Pub. No.: US 2024/0043860 A1
(43) Pub. Date: Feb. 8, 2024

(54) NOVEL HYDROXYPHENYLPYRUVATE DIOXYGENASE POLYPEPTIDES AND METHODS OF USE THEREOF

(71) Applicant: Syngenta Crop Protection AG, Basel (CH)

(72) Inventors: Richard Dale, Bracknell, Berkshire (GB); Marta Andreia Horta Simoes, Bracknell, Berkshire (GB); Rachael Elizabeth Blain, Bracknell, Berkshire (GB); Fiona Callaghan, Bracknell, Berkshire (GB)

(73) Assignee: Syngenta Crop Protection AG, Basel (CH)

(21) Appl. No.: 18/254,698

(22) PCT Filed: Nov. 17, 2021
(86) PCT No.: PCT/US21/59714

§ 371 Date: May 26, 2023

Related U.S. Application Data
(60) Provisional application No. 63/119,226, filed on Nov. 30, 2020.

(51) Int. Cl. C12N 15/82; C12N 9/02; C07K 14/415

(52) U.S. Cl. CPC C12N 15/8274; C12N 9/0069; C07K 14/415; C12Y 113/11027

(57) ABSTRACT

Novel hydroxyphenyl pyruvate dioxygenase (HPPD) polypeptides, variants and fragments thereof, as well as polynucleotides encoding the same, capable of conferring commercial levels of conferring HPPD herbicide resistance or tolerance to plants. Compositions include amino acid sequences, and variants and fragments thereof, for HPPD polypeptides, as well as polynucleotides encoding the same. Methods for the production and use of HPPD herbicide resistant plants that express these novel HPPD polypeptides, methods for selectively controlling weeds in a field at a crop locus, and methods for characterization, identification and selection of these novel HPPDs are also provided.

### RELATED APPLICATION

This application claims priority to U.S. Patent Application No. 63/119226, filed 30 Nov. 2020, the entire contents of which are incorporated by reference herein.

### TECHNICAL FIELD

The present disclosure relates to novel hydroxyphenyl pyruvate dioxygenase (HPPD) polypeptides that confer herbicide resistance or tolerance to plants and the nucleic acid sequences that encode them. Methods of the invention relate to the production and use of plants that express these mutant HPPD polypeptides and that are resistant to HPPD herbicides.

### SEQUENCE LISTING

This application is accompanied by a sequence listing entitled 82212-WO-HPPD_ST25.txt, created on Nov. 12, 2021 and approximately ˜612 kb in size, and which is incorporated by reference herein in its entirety. This sequence listing is submitted herewith via EFS-Web, and is in compliance with 37 C.F.R. § 1.824(a)(2)-(6) and (b). clp BACKGROUND

The hydroxyphenylpyruvate dioxygenases (HPPDs) are enzymes that catalyze the reaction in which para-hydroxyphenylpyruvate (HPP) is transformed into homogentisate. This reaction takes place in the presence of enzyme-bound iron (Fe²⁺) and oxygen. Herbicides that act by inhibiting HPPD are well known, and include isoxazoles, diketonitriles, triketones, and pyrazolinates (Hawkes “Hydroxyphenylpyruvate Dioxygenase (HPPD)—The Herbicide Target.” In Modern Crop Protection Compounds. Eds. Krämer and Schirmer. Weinheim, Germany: Wiley-VCH, 2007. Ch. 4.2, pp. 211-220). Inhibition of HPPD blocks the biosynthesis of plastoquinone (PQ) from tyrosine. PQ is an essential cofactor in the biosynthesis of carotenoid pigments which are essential for photoprotection of the photosynthetic centres. HPPD-inhibiting herbicides are phloem-mobile bleachers which cause the light-exposed new meristems and leaves to emerge white. In the absence of carotenoids, chlorophyll is photo-destroyed and becomes itself an agent of photo-destruction via the photo-generation of singlet oxygen.

Methods for providing plants that are tolerant to HPPD herbicides are also known. These methods have included: 1) overexpressing the HPPD enzyme so as to produce quantities of HPPD enzyme in the plant that are sufficient in relation to a given herbicide so as to have enough of the functional enzyme available for the plant to thrive despite the presence of the herbicide; and 2) mutating a particular HPPD enzyme into an enzyme that is less sensitive to inhibition by herbicides. Methods for mutating HPPD enzymes for improved HPPD herbicide tolerance have been described (see, e.g., PCT Application Nos. WO 99/24585 and WO 2009/144079), and some particular mutations of plant HPPD enzymes (e.g., mutation of G422 in the Arabidopsis HPPD sequence) are purportedly capable of providing some measure of tolerance to mesotrione and other triketone herbicides. However, the enzyme kinetics and whole plant data reported thus far are insufficient to conclude whether the reported mutational changes confer commercially significant benefits over the corresponding wild type enzyme(s).

Furthermore, while a particular HPPD enzyme may provide a useful level of tolerance to some HPPD-inhibitor herbicides, the same HPPD may be quite inadequate to provide commercial levels of tolerance to a different, more desirable HPPD-inhibitor herbicide (See, e.g., U.S. Patent Application Publication No. 20040058427; PCT Publication Nos. WO 98/20144 and WO 02/46387; see also U.S. Patent Application Publication No. 20050246800 relating to the identification and labelling of soybean varieties as being relatively HPPD tolerant). Moreover, applicant desires mutated versions of HPPDs from cool-climate grasses with improved resistance. Such mutants would be highly desirable, as HPPDs from cool-climate grasses are likely preferable to other types in some situations (see, e.g., PCT Application No. WO 02/46387 and Hawkes et al. 2001 in Proc. Brit. Crop Prot. Conf. Weeds 2, 563). Accordingly, new methods and compositions for conferring commercial levels of HPPD herbicide tolerance upon various crops and crop varieties are needed.

### SUMMARY

Compositions and methods for conferring hydroxyphenyl pyruvate dioxygenase (HPPD) herbicide resistance or tolerance to plants are provided. The compositions include nucleotide and amino acid sequences for HPPD polypeptides. In certain embodiments, the polypeptides of the invention include novel HPPDs derived from plants that confer resistance or tolerance when expressed heterologously in other plants to certain classes of herbicides that inhibit HPPD. In particular embodiments, these HPPDs comprise amino acid sequences set forth in SEQ ID NOs: 4 to 63 and 122 to 125, and novel polypeptides having at least about 99, 98, 97, 96, 95, 94, 93, 92, 91 or 90% sequence identity to any of SEQ ID NOs: 4-63 and 122-125 and that exhibit HPPD enzyme activity.

Exemplary novel HPPDs are likewise those that, in comparison with HPPD enzymes of the prior art, exhibit superior tolerance to one or more types of HPPD herbicide and where tolerance is characterised in vitro by the numerical value of the parameter (k_{off}×k_{cat}/K_{m HPP}) and where k_{off} is the rate constant governing the dissociation rate of the complex of the HPPD enzyme with herbicide and k_{cat}/K_{m HPP} is the catalytic turnover number divided by the Kₘ value for the substrate HPP (4-hydroxyphenyl pyruvate).

In a further embodiment of the current invention there is therefore also provided an in vitro method for characterising and selecting HPPDs that confer superior levels of tolerance to HPPD herbicides based on measuring and comparing values of k_{cat}/K_{m HPP} and k_{off} or functional equivalents of these parameters.

In further embodiments the polypeptides of the invention are catalytically active mutant HPPDs that derive from plants and that, relative to the like unmutated enzyme, confer superior levels of resistance or tolerance to certain classes of herbicides that inhibit HPPD. In particular embodiments, these mutant HPPD polypeptides comprise one or more amino acid sequences selected from SEQ ID NOs: 59-63, wherein SEQ ID NOs: 59-63 have one or more amino acid substitutions described as follows, and wherein the position of the amino acid substitutions of SEQ ID Nos: 59-63 are based on the alignment of the sequences with the reference sequence SEQ ID NO: 1:

| | | |
|---|---|---|
| SEQ ID | R214; X1, X2, X3, X4, | X1 = Y or F; X2 = G; X3 = L or I; |
| NO: 59 | X5, X6, X7, X8, X9 | X4 = T, Q, S or R; X5 = G; |
| | | X6 = F or L; X7 = D; |
| | | X8 = H; X9 = V, A, I or C |
| SEQ ID | V260; X1, X2, X3, X4, | X1 = L; X2 = N; X3 = S; X4 = V, |
| NO: 60 | X5, X6, X7, X8, X9 | A or M; X5 = A or T; |
| | | X6 = L; X7 = A; |
| | | X8 = N, S or C; X9 = N or T |
| SEQ ID | P271; X1, X2, X3, X4, | X1 = A, M, G, R, N, T; X2 = V; |
| NO: 61 | X5, X6, X7, X8, X9 | X3 = L or P; X4 = L, I or F; |
| | | X5 = N; X6 = L, M, I or V; |
| | | X7 = N; X8 = E; X9 = E |
| SEQ ID | S304; X1, X2, X3, X4, | X1 = I, L or M; X2 = A; X3 = L |
| NO: 62 | X5, X6, X7, X8, X9 | or V; X4 = A, M, K, L, S or V; |
| | | X5 = T; X6 = S, E, N, D, H or R; |
| | | X7 = D, E or N; |
| | | X8 = V or I; X9 = F, I or L |
| SEQ ID | K404; X1, X2, X3, X4, | X1 = Q, E, T or K; X2 = E, A, |
| NO: 63 | X5, X6, X7, X8, X9 | M or V; X3= Y, Q or A; |
| | | X4 = Q, G or A; X5 =N; X6 = G; |
| | | X7 = Q, C, G or A; |
| | | X8 = C or G; X9 = G, or L |

In some embodiments the mutant HPPD may be derived from a monocot plant and, in particular, a cool climate grass species such as wheat, barley, oats or rye. In some embodiments the mutant HPPD may be derived from Lolium, Apera, Setaria, Avena, Poa, Alopecurus or Sorghum species. In some embodiments, the mutant HPPD may be derived from Avena, Apera, or Alopecurus species. Exemplary species include Avena sativa, Apera spica, and Alopecurus myosuroides. In one embodiment, the mutant HPPD may be derived from one or more of the HPPD polypeptides of SEQ ID NOs: 1-3.

Exemplary HPPD polypeptides and mutant HPPD polypeptides according to the invention correspond to the amino acid sequences set forth in SEQ ID NOs: 4-63 and 122 to 125 and variants and functional fragments thereof. Nucleic acid molecules comprising polynucleotide sequences that encode these particular HPPD polypeptides of the invention are further provided, e.g., at SEQ ID NOs: 64-118 and 128-133. Compositions also include expression cassettes comprising a promoter operably linked to a nucleotide sequence that encodes an HPPD polypeptide of the invention, alone or in combination with one or more additional nucleic acid molecules encoding polypeptides that confer desirable traits, e.g., SEQ ID NOs: 119-121. Transformed plants, plant cells, and seeds comprising an expression cassette of the invention are further provided. Embodiments of the invention may also include compositions and methods for editing endogenous polynucleotide sequences that encode the particular HPPD polypeptides disclosed herein.

The compositions of the invention are useful in methods directed to conferring herbicide resistance or tolerance to plants, particularly resistance or tolerance to certain classes of herbicides that inhibit HPPD. In particular embodiments, the methods comprise introducing into a plant at least one expression cassette comprising a promoter operably linked to a nucleotide sequence that encodes an HPPD polypeptide of the invention. As a result, the HPPD polypeptide is expressed in the plant, and since the HPPD is selected on the basis that it is less sensitive to HPPD-inhibiting herbicides, this leads to the plant exhibiting substantially improved resistance or tolerance to HPPD-inhibiting herbicides.

Methods of the present invention also comprise selectively controlling weeds in a field at a crop locus. In one embodiment, such methods involve over-the-top pre- or postemergence application of weed-controlling amounts of HPPD herbicides in a field at a crop locus that contains plants expressing the HPPD polypeptides of the invention. In other embodiments, methods are also provided for the assay, characterization, identification, and selection of the HPPDs of the current invention.

### BRIEF DESCRIPTION OF THE SEQUENCE LISTING

SEQ ID NO: 1 is the native HPPD protein from Avena sativa.

SEQ ID NO: 2 is the native HPPD protein from Apera speca-venti.

SEQ ID NO: 3 is the native HPPD protein from Alopecurus myosuroides.

SEQ ID NO: 4 is an artificial oat HPPD protein created to include a deletion at A111 relative to the native oat HPPD protein of SEQ ID NO: 1.

SEQ ID NOs: 5-58 are artificial mutated HPPD proteins comprising amino acid sequences modified with modification positions indicated relative to the corresponding native HPPD protein.

SEQ ID NOs: 59-63 are artificial HPPD polypeptide motifs.

SEQ ID NOs: 64-118 are artificial polynucleotide sequences encoding for the mutated HPPD proteins of SEQ ID NOs: 4-58, respectively.

SEQ ID NOs: 119-121 are artificial DNA sequences corresponding to a vector for expressing the mutated HPPD proteins of SEQ ID NOs: 11, 14, and 17, respectively.

SEQ ID NOs: 122-127 are artificial mutated HPPD proteins comprising amino acid sequences modified with modification positions indicated relative to the corresponding native HPPD protein.

SEQ ID NOs: 128-133 are artificial polynucleotide sequences encoding for the mutated HPPD proteins of SEQ ID NOs: 122-127, respectively.

SEQ ID NOs: 134-188 are protein sequences from variants, homologues, orthologues and paralogues of HPPD polypeptides.

### BRIEF SUMMARY OF THE DRAWINGS

FIG. 1 shows a representation of binary vector pBinAvenaSativaHPPDV207 for plant transformation conferring HPPD resistance with a mutated HPPD gene encoding the amino acid sequence set forth in SEQ ID NO: 11.

FIG. 2 shows a representation of binary vector pBinAvenaSativaHPPDV208 for plant transformation conferring HPPD resistance with a mutated HPPD gene encoding the amino acid sequence set forth in SEQ ID NO: 14 and also conferring tolerance to glyphosate (selectable marker).

FIG. 3 shows a representation of binary vector pBinAvenaSativaHPPDV209 for plant transformation conferring HPPD resistance with a mutated HPPD gene encoding the amino acid sequence set forth in SEQ ID NO: 17 and also conferring tolerance to glyphosate (selectable marker).

FIG. 4 shows a transgenic plant exhibiting improved resistance to an HPPD-inhibiting herbicide due to the introduction into the plant of a mutated HPPD gene encoding the amino acid sequence set forth in SEQ ID NO: 14.

FIG. 5 shows the higher relative tolerance of transgenic plants expressing a mutated HPPD gene encoding the amino acid sequence set forth in SEQ ID NO: 14 to an HPPD-inhibiting herbicide relative to transgenic plants expressing other mutated HPPD genes.

### DETAILED DESCRIPTION

The present invention provides compositions and methods directed to conferring hydroxyphenyl pyruvate dioxygenase (HPPD) herbicide resistance or tolerance to plants. Compositions include amino acid sequences for native and mutant HPPD polypeptides having HPPD enzymatic activity, and variants and functional fragments thereof. Nucleic acids that encode the mutant HPPD polypeptides of the invention are also provided. Methods for conferring herbicide resistance or tolerance to plants, particularly resistance or tolerance to certain classes of herbicides that inhibit HPPD, are further provided. Methods are also provided for selectively controlling weeds in a field at a crop locus and for the assay, characterization, identification and selection of the mutant HPPDs of the current invention that provide herbicide tolerance.

Within the context of the present invention the terms hydroxy phenyl pyruvate dioxygenase (HPPD), 4-hydroxy phenyl pyruvate dioxygenase (4-HPPD) and p-hydroxy phenyl pyruvate dioxygenase (p-HPPD) are synonymous.

“HPPD herbicides” are herbicides that are bleachers and whose primary site of action is HPPD. Many are well known and described elsewhere herein and in the literature (Hawkes “Hydroxyphenylpyruvate Dioxygenase (HPPD)—The Herbicide Target.” In Modern Crop Protection Compounds. Eds. Kramer and Schirmer. Weinheim, Germany: Wiley-VCH, 2007. Ch. 4.2, pp. 211-220; Edmunds “Hydroxyphenylpyruvate dioxygenase (HPPD) Inhibitors: Triketones.” In Modern Crop Protection Compounds. Eds. Kramer and Schirmer. Weinheim, Germany: Wiley-VCH, 2007. Ch. 4.2, pp. 221-242). As used herein, the term “HPPD herbicides” refers to herbicides that act, either directly or indirectly, to inhibit HPPD, where the herbicides are bleachers, and where inhibition of HPPD is at least part of the herbicide's mode of action on plants.

As used herein, when treated with said herbicide, plants which are substantially “tolerant” to a herbicide exhibit a dose/response curve which is shifted to the right when compared with the dose/response curve exhibited by similarly subjected non-tolerant like plants. Such dose/response curves have “dose” plotted on the x-axis and “percentage kill or damage”, “herbicidal effect”, etc., plotted on the y-axis. As understood by people in the art, a shift to the right on the dose/response curve implies that a higher dose of the substance is required to produce a similar response. Tolerant plants will typically require at least twice as much herbicide as non-tolerant like plants in order to produce a given herbicidal effect. Plants which are substantially “resistant” to the herbicide exhibit few, if any, necrotic, lytic, chlorotic or other lesions or, at least, none that significantly impact plant yield, when subjected to the herbicide at concentrations and rates which are typically employed by the agricultural community to kill weeds in the field.

As used herein, “non-transgenic-like plants” are plants that are similar to or the same as transgenic plants but that do not contain a transgene conferring herbicide resistance.

As used herein, the term “confer” refers to providing a characteristic or trait, such as herbicide tolerance or resistance and/or other desirable traits to a plant.

As used herein, “heterologous” in reference to a sequence is a sequence that originates from a foreign species, or, if from the same species, is substantially modified from its native form in composition and/or genomic locus by deliberate human intervention. As such, “heterologous” refers to, when used in reference to a gene or nucleic acid, a gene encoding a factor that is not in its natural environment (i.e., has been altered by the of man). For example, a heterologous gene may include a gene from one species introduced into another species. A heterologous gene may also include a gene native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an antisense RNA transcript that is complementary to the mRNA transcript). In one aspect of the invention, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, in embodiments, a “heterologous” polynucleotide is a polynucleotide not naturally associated with a host cell into which it is introduced, including non-naturally occurring multiple copies of a naturally occurring polynucleotide.

As used herein, a chimeric gene comprises a coding sequence operably linked to a transcription initiation region that is heterologous to the coding sequence. In specific embodiments the term “heterologous” means from another source, such as from another organism or another plant (such as another plant of a different species or the same species). In the context of DNA, “heterologous” refers to any foreign “non-self” DNA including that from another plant of the same species.

The article “a” and “an” are used herein to refer to one or more than one (i.e., to at least one) of the grammatical object of the article. By way of example, “an element” means one or more of the element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

A variety of additional terms are defined or otherwise characterized herein.

### HPPD Sequences

The compositions disclosed herein include isolated or substantially purified HPPD polynucleotides and HPPD polypeptides as well as host cells comprising the HPPD polynucleotides and expressing the encoded HPPD polypeptides. Specifically, the present invention provides HPPD polypeptides that have HPPD enzymatic activity and that confer enhanced resistance or tolerance in plants to certain classes of herbicides that inhibit HPPD, and variants and fragments thereof. Nucleic acids that encode HPPD polypeptides of the invention are also provided.

Mutant HPPD polypeptides of the present disclosure have amino acid changes at one or more positions relative to the native wild-type HPPD enzyme sequence from which they are derived, and exhibit enhanced tolerance to one or more HPPD inhibitor herbicides relative to their unmutated counterparts as well as relative to other mutant HPPDs. HPPD enzymes that exhibit enhanced tolerance to an HPPD herbicide may do so by virtue of exhibiting, relative to the like unmutated native HPPD enzyme:

a) a lower Kₘ value for the natural substrate, 4-hydroxyphenylpyruvate;
b) a higher k_{cat} value for converting 4-hydroxyphenylpyruvate to homogentisate;
c) a lower value of the apparent rate constant, kₒₙ, governing formation of an enzyme: HPPD inhibitor herbicide complex;
d) an increased value of the rate constant, k_{off}, governing dissociation of an enzyme: HPPD inhibitor herbicide complex; and/or
e) as a result of changes in one or both of c) and d), an increased value of the equilibrium constant, Kᵢ (also called K_{d}), governing dissociation of an enzyme: HPPD inhibitor herbicide complex. DNA sequences encoding such improved mutated HPPDs are used in the provision of HPPD plants, crops, plant cells and seeds of the current invention that offer enhanced tolerance or resistance to one or more HPPD herbicides as compared to like plants expressing the unmutated native enzyme.

Thus exemplary HPPDs are selected as those that, in comparison with other HPPD enzymes (such as other mutant HPPDs or other native HPPDs), exhibit superior tolerance to one or more types of HPPD herbicide and where tolerance is characterised in vitro by the numerical value of the parameter (k_{off}×k_{cat}/K_{m HPP}) and where k_{off} is the rate constant governing the dissociation rate of the complex of the HPPD enzyme with herbicide and k_{cat}/K_{m HPP} is the catalytic turnover number divided by the Kₘ value for the substrate HPP (4-hydroxyphenylpyruvate).

Thus in one embodiment of the current disclosure there is provided an in vitro method for characterising and selecting HPPDs that confer superior levels of tolerance to HPPD herbicides based on measuring and comparing values of k_{cat}/K_{m HPP} and k_{off} or functional equivalents of these parameters.

The present invention also provides plants transformed with at least one expression cassette encoding a mutant HPPD polypeptide of the present invention that have HPPD enzymatic activity and that confer resistance or tolerance in the transformed plant to certain classes of herbicides that inhibit HPPD. Transformed plants of the present disclosure exhibit phenotypes associated with tolerance to one or more HPPD inhibitor herbicides including one or more of reduced chlorosis, reduced necrosis, reduced stunting, reduced lesion formation, etc.

Site-directed mutations of genes encoding HPPDs is performed wherein mutations are selected so as to encode one or more amino acid changes selected from those listed here, such as single mutations or combinations of mutations. Genes encoding such mutant forms of HPPDs are useful for making crop plants resistant to herbicides that inhibit HPPD. HPPD genes so modified are especially suitable for use in transgenic plants in order to confer herbicide tolerance or resistance upon crop plants. In one embodiment of the present disclosure, a method of improving plant yield is provided comprising selectively growing in a field a plant comprising a mutant HPPD of the present invention, and applying to the field, over-the-top pre- or postemergence, a weed-controlling amount of an HPPD herbicide to which the transgenic plant has enhanced resistance or tolerance.

Example methods of modifying native HPPD sequences to generate mutant HPPD polypeptides that exhibit herbicide tolerance are also disclosed, for example, in PCT Pub. Nos. WO2010/085705 and WO2011/068567, the contents of which are incorporated by reference herein in its entirety.

Many HPPD sequences are known in the art and can be used to generate mutant HPPD sequences by making amino acid substitutions corresponding to those described herein. In typical embodiments the HPPDs used herein are derived from plants. For example, the sequence to be improved by mutation can be aligned with, for example, any one of the native HPPD sequences of SEQ ID NOS: 1-4 (representing the native HPPD of Avena sativa, Apera spica-venti, and Alopecurus myosuroides, respectively) using standard sequence alignment tools such as BLASTP, and the corresponding amino acid substitutions described herein with respect to SEQ ID NOs: 1-4 can be made at positions corresponding to the positions in the reference sequence.

In other examples, a known or suspected HPPD sequence can be inspected for the presence of the amino acid motifs of SEQ ID NOs: 59-63 and the corresponding changes described herein can be made. In the case of HPPDs not deriving from plants the equivalent changes to those indicated here can be made on the basis of multiple sequence alignments and similarity to the motifs specified here.

All of the compositions, methods, plants, plant parts, and expression cassettes disclosed herein can include mutant HPPD polypeptides derived from any of the HPPD sequences listed herein, including those listed at Tables 1.1, 1.2, and 1.3. Further, the mutant HPPD polypeptides comprise amino acid substitution(s) corresponding to the amino acid positions listed with reference to any of Tables 1.1, 1.2. and 1.3.

In particular embodiments, the compositions of the invention comprise a mutant HPPD polypeptide having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to SEQ ID NO: 1 (the HPPD amino acid sequence of Avena sativa) or SEQ ID NO: 2 (the HPPD amino acid sequence of Apera spica-venti), where the polypeptide has HPPD enzymatic activity, and where the polypeptide contains one or more substitution(s) corresponding to the amino acid positions of SEQ ID NO: 1 or 2 or 3 listed in column 1 of Table 1.1.

In particular embodiments, the compositions provided comprise a mutant HPPD polypeptide derived from any one of SEQ ID NOs 1, 2, 3, and 4, wherein the polypeptide comprises amino acid substitution(s) corresponding to the amino acid positions listed in column 1 of Table 1.1, such as at least one, at least two, at least three, at least four, at least five, at least six, at least seven, at least eight, at least nine, or at least ten substitutions, and where the polypeptide has HPPD enzymatic activity.

TABLE 1.1
Exemplary HPPD mutations

| Mutatable amino acid position relative | |
|---|---|
| to SEQ ID NO: 1 or SEQ ID NO: | Exemplary Substitution or |
| 2 or SEQ ID NO: 3 | addition |
| 214 | G |
| 218 | I |
| 260 | A |
| 271 | N |
| 304 | T |
| 327 | R |
| 340 | E |
| 359 | M or Y |
| 368 | M |
| 411 | A |

Table 1.2 shows an overview of exemplary mutation sites that are shared between a non-limiting list of variants, homologues, orthologues and paralogues of HPPD polypeptides.

| SEQ ID | | | | | | | | | | |
|---|---|---|---|---|---|---|---|---|---|---|
| NO | Pos 1 | Pos 2 | Pos 3 | Pos 4 | Pos 5 | Pos 6 | Pos 7 | Pos 8 | Pos 9 | Pos 10 |
| 1 | R214 | V218 | V260 | P271 | S304 | A327 | I340 | L359 | K404 | G411 |
| 2 | R214 | V218 | V260 | P271 | S304 | A327 | L340 | L359 | K404 | G411 |
| 3 | R214 | V218 | V260 | P271 | S304 | A327 | L340 | L359 | K404 | G411 |
| 4 | R213 | V217 | V259 | P270 | S303 | A326 | I339 | L358 | K403 | G410 |
| 134 | R210 | V214 | V256 | P267 | S300 | P323 | I336 | L355 | K400 | G407 |
| 135 | R214 | A218 | V260 | P271 | S304 | P327 | R340 | L359 | K404 | G411 |
| 136 | R223 | A227 | V269 | P280 | S313 | P336 | R349 | L368 | S413 | G420 |
| 137 | R219 | A223 | V265 | P276 | S309 | P332 | R345 | L364 | S409 | G416 |
| 138 | R210 | V214 | V256 | P267 | S300 | A323 | R336 | L355 | K400 | G407 |
| 139 | R218 | A222 | V264 | P275 | S308 | P331 | R344 | L363 | S408 | G415 |
| 140 | R222 | A226 | V268 | P279 | S312 | P335 | R348 | L367 | S412 | G419 |
| 141 | R221 | A225 | V267 | P278 | S311 | P334 | R347 | L366 | S411 | G418 |
| 142 | R208 | V212 | V254 | P265 | S298 | P321 | L334 | L353 | K398 | G405 |
| 143 | R228 | A232 | V274 | P285 | S318 | P341 | R354 | L373 | S418 | G425 |
| 144 | R224 | A228 | V270 | P281 | S314 | P337 | R350 | L369 | S414 | G421 |
| 145 | R209 | A213 | V255 | P266 | S299 | P322 | R335 | L354 | A405 | G412 |
| 146 | R205 | A209 | V251 | P262 | T295 | S318 | K331 | L350 | A435 | G442 |
| 147 | R226 | A230 | V272 | P283 | S316 | A339 | R352 | L371 | K416 | G423 |
| 148 | R216 | A220 | V262 | P273 | S306 | P329 | R342 | L361 | K406 | G413 |
| 149 | R213 | I217 | V259 | P270 | S303 | A326 | R339 | L358 | K403 | G410 |
| 150 | R217 | I221 | V263 | P274 | S307 | E330 | R343 | L362 | K407 | G414 |
| 151 | R214 | I218 | V260 | P271 | S304 | A327 | R340 | L359 | K404 | G411 |
| 152 | R224 | A228 | V270 | P281 | S314 | P337 | R350 | L369 | S414 | G421 |
| 153 | R269 | A273 | V315 | P326 | T359 | P382 | R395 | L414 | K459 | G466 |
| 154 | R213 | A217 | V259 | P270 | S303 | P326 | R339 | L358 | K403 | G410 |
| 155 | R227 | A231 | V273 | P284 | S317 | P340 | R353 | L372 | K417 | G424 |
| 156 | R208 | V212 | V254 | P265 | S298 | P321 | L334 | L353 | K398 | G405 |
| 157 | R216 | A220 | V262 | P273 | S306 | P329 | R342 | L361 | K406 | G413 |
| 158 | R222 | A226 | V268 | P279 | S312 | P335 | R348 | L367 | K412 | G419 |
| 159 | R219 | V223 | V265 | P276 | S309 | A332 | I345 | L364 | K409 | G416 |
| 160 | R231 | A235 | V277 | P288 | S321 | P344 | R357 | L376 | K421 | G428 |
| 161 | R221 | A225 | V267 | P278 | S311 | P334 | R347 | L366 | S411 | G418 |
| 162 | R213 | I217 | V259 | P270 | S303 | A326 | R339 | L358 | K403 | G410 |
| 163 | R219 | A223 | V265 | P276 | S309 | P332 | R345 | L364 | K409 | G416 |
| 164 | R221 | A225 | V267 | P278 | T311 | P334 | R347 | L366 | K411 | G418 |
| 165 | R215 | V219 | V261 | P272 | S305 | A328 | R341 | L360 | K405 | G412 |
| 166 | R212 | V216 | V258 | P269 | S302 | A325 | R338 | L357 | K402 | G409 |
| 167 | R220 | V224 | V266 | P277 | S310 | A333 | R346 | L365 | K410 | G417 |
| 168 | R213 | V217 | V259 | P270 | S303 | A326 | R339 | L358 | K403 | G410 |
| 169 | R213 | V217 | V259 | P270 | S303 | A326 | R339 | L358 | K403 | G410 |
| 170 | R213 | I217 | V259 | P270 | S303 | A326 | R339 | L358 | K403 | G410 |
| 171 | R225 | A229 | V271 | P282 | S315 | A338 | R351 | L370 | K415 | G422 |
| 172 | R219 | A223 | V265 | P276 | S309 | P332 | R345 | L364 | S409 | G416 |
| 173 | R215 | A219 | V261 | P272 | S305 | P328 | R341 | L360 | K405 | G412 |
| 174 | R199 | V203 | V245 | P256 | S289 | A312 | R325 | L344 | R389 | G396 |
| 175 | R198 | I202 | V244 | P255 | S288 | A311 | R324 | L343 | R388 | G395 |
| 176 | R221 | A225 | V267 | P278 | S311 | P334 | R347 | L366 | S411 | G418 |
| 177 | R214 | I218 | V260 | P271 | S304 | A327 | R340 | L359 | K404 | G411 |
| 178 | R218 | I222 | V264 | P275 | S308 | A331 | R344 | L363 | K408 | G415 |
| 179 | H165 | V169 | V213 | P224 | T256 | D275 | R287 | L304 | — | G341 |
| 180 | G140 | V144 | V185 | P196 | T226 | P245 | R257 | L276 | R310 | G317 |
| 181 | R213 | A217 | V259 | P270 | S303 | P326 | R339 | L358 | K403 | G410 |
| 182 | R210 | V214 | V256 | P267 | S300 | P323 | I336 | L355 | K400 | G407 |
| 183 | R210 | V214 | V256 | P267 | S300 | P323 | I336 | L355 | K400 | G407 |
| 184 | R207 | V211 | V253 | P264 | S297 | P320 | I333 | L352 | K397 | G404 |
| 185 | R211 | A215 | V257 | P268 | S301 | P324 | R337 | L356 | K401 | G408 |
| 186 | R215 | I219 | V261 | P272 | S305 | A328 | R341 | L360 | K405 | G412 |
| 187 | R223 | A227 | V269 | P280 | S313 | P336 | R349 | L368 | K413 | G420 |
| 188 | R212 | I216 | V258 | P269 | S302 | A325 | R338 | L357 | K402 | G409 |

In non-limiting embodiments, an amino acid at one or more position(s) listed in column 1 of Table 1.1 or as listed in Table 1.2 is replaced with any other amino acid. In another embodiment, the polypeptide comprises one or more amino acid substitutions, additions, or deletions corresponding to the amino acid substitution(s) or deletion(s) listed in Tables 1.1-1.2. In yet another embodiment, the polypeptide comprises one or more substitutions corresponding to a conservative variant of the amino acids listed in column 2 of Table 1.1. In particular embodiments, the compositions of the invention comprise a mutant HPPD polypeptide derived from any one of SEQ ID NOs 1-4, wherein the polypeptide contains one to ten amino acid substitution(s) corresponding to the amino acid substitutions listed in column 2 of Table 1.1, such as at least one, at least two, at least three, at least four, at least five, at least six, at least seven, at least eight, at least nine, or at least ten substitutions, and where the polypeptide has HPPD enzymatic activity. Table 1.3 shows non-limiting example combinations of 2, 3, 4, 5, 6, 7, 8, 9, and 10 mutations wherein each mutatable position is with reference to SEQ ID NOS: 1-3.

TABLE 1.3
Exemplary HPPD mutation combinations

| Number of | Combination of Mutatable amino acid positions relative to any of |
|---|---|
| mutations | SEQ ID NOS: 1-3 |
| 2 | 214/218; 214/327; 214/340; 214/359; 214/411; 218/327; 218/340; 218/359; |
| | 218/411; 327/340; 327/359; 327/411; 340/359; 340/411; 359/411; 214/260; |
| | 214/271; 214/304; 214/404; 218/260; 218/271; 218/304; 218/404; 327/260; |
| | 327/271; 327/304; 327/404; 340/260; 340/271; 340/304; 340/404; 359/260; |
| | 359/271; 359/304; 359/404; 411/260; 411/271; 411/304; 411/404; 260/271; |
| | 260/304; 260/404; 217/304; 271/404; 304/404 |
| 3 | 214/218/327; 214/218/340; 214/218/359; 214/218/411; 214/218/260; 214/218/271; |
| | 214/218/304; 214/218/404; 214/327/340; 214/327/359; 214/327/411; 214/327/260; |
| | 214/327/271; 214/327/304; 214/327/404; 214/340/359; 214/340/411; 214/340/260; |
| | 214/340/271; 214/340/304; 214/340/404; 214/359/411; 214/359/260; 214/359/271; |
| | 214/359/304; 214/359/404; 214/411/260; 214/411/271; 214/411/304; 214/411/404; |
| | 214/260/271; 214/260/304; 214/260/404; 214/271/304; 214/271/404; 214/304/404 |
| 4 | 214/218/260/271; 214/218/260/304; 214/218/260/327; 214/218/260/340; |
| | 214/218/260/359; 214/218/260/404; 214/218/260/411; 214/260/271/304; |
| | 214/260/271/327; 214/260/271/340; 214/260/271/359; 214/260/271/404; |
| | 214/260/271/411; 214/218/271/304; 214/218/271/327; 214/218/271/340; |
| | 214/218/271/359; 214/218/271/404; 214/218/271/411; 218/260/271/304; |
| | 218/260/271/327; 218/260/271/340; 218/260/271/359; 218/260/271/404; |
| | 218/260/271/411; 260/271/304/327; 260/271/304/340; 260/271/304/359; |
| | 260/271/304/404; 260/271/304/411; 260/271/304/218; 271/304/327/340; |
| | 271/304/327/359; 271/304/327/404; 271/304/327/411; 271/304/327/214; |
| | 271/304/327/218; 304/327/340/359; 304/327/340/404; 304/327/340/411; |
| | 304/327/340/214; 304/327/340/218; 304/327/340/260; 327/340/359/404; |
| | 327/340/359/411; 327/340/359/214; 327/340/359/218; 327/340/359/271; |
| | 340/359/404/411; 340/359/404/201; 340/359/404/218; 340/359/271; |
| | 340/359/404/304; 359/404/411/214; 359/404/411/218; 359/404/411/271; |
| | 359/404/411/304; 359/404/411/327 |
| 5 | 214/218/260/271/304; 214/218/260/271/327; 214/218/260/271/340; |
| | 214/218/260/271/359; 214/218/260/271/404; 214/218/260/271/411; |
| | 218/260/271/304/327; 218/260/271/304/340; 218/260/271/304/404; |
| | 218/260/271/304/411; 260/271/304/327/340; 260/271/304/327/359; |
| | 260/271/304/327/404; 260/271/304/327/411; 271/304/327/340/359; |
| | 271/304/327/340/404; 271/304/327/340/411; 304/327/340/359/404; |
| | 304/327/340/359/411; 304/327/340/359/214; 304/327/340/359/218; |
| | 304/327/340/359/404/260 |
| 6 | 214/218/260/271/304/327; 214/218/260/271/304/340; 214/218/260/271/304/359; |
| | 214/218/260/271/304/404; 214/218/260/271/304/411; 214/260/271/304/327/359; |
| | 214/260/271/304/327/404; 214/260/271/304/327/411; 214/218/271/304/327/340; |
| | 214/218/271/304/327/404; 214/218/271/304/327/411; 214/218/260/304/327/340; |
| | 214/218/260/304/327/359; 214/218/260/304/327/404; 214/218/260/304/327/411; |
| | 214/218/260/271/327/340; 214/218/260/271/327/359; 214/218/260/271/327/404; |
| | 214/218/260/271/327/411; 214/218/260/271/304/340; 214/218/260/271/304/359; |
| | 214/218/260/271/304/404; 214/218/260/271/304/411; 218/260/271/304/327/340; |
| | 218/260/271/304/327/359; 218/260/271/304/327/404; 218/260/271/304/327/411 |
| 7 | 214/218/260/271/304/327/340; 214/218/260/271/304/327/359; |
| | 214/218/260/271/304/327/404; 214/218/260/271/304/327/411; |
| | 218/260/271/304/327/340/359; 218/260/271/304/327/340/404; |
| | 218/260/271/304/327/340/411; 260/271/304/327/340/359/404; |
| | 260/271/304/327/340/359/411; 271/304/327/340/359/404/411; |
| | 214/260/271/304/327/340/359; 214/260/271/304/327/340/404; |
| | 214/260/271/304/327/340/411; 214/218/271/304/327/340/359; |
| | 214/218/271/304/327/340/404; 214/218/271/304/327/340/411; |
| | 214/218/271/304/327/340/359; 214/218/271/304/327/340/404; |
| | 214/218/271/304/327/340/411; 214/218/260/304/327/340/359; |
| | 214/218/260/304/327/340/404; 214/218/260/304/327/340/411; |
| | 214/218/260/271/327/340/359; 214/218/260/271/327/340/404; |
| | 214/218/260/271/327/340/411; 214/218/260/271/304/340/359; |
| | 214/218/260/271/304/340/404; 214/218/260/271/304/340/411 |
| 8 | 214/218/260/271/304/327/340/359; 214/218/260/271/304/327/340/404; |
| | 214/218/260/271/304/327/340/411; 214/218/260/271/304/327/404/411; |
| | 214/260/271/304/327/340/359/404; 214/260/271/304/327/340/359/411; |
| | 214/218/271/304/327/340/359/404; 214/218/271/304/327/340/359/404; |
| | 214/218/260/304/327/340/359/404; 214/218/260/304/327/340/359/404; |
| | 214/218/260/271/327/340/359/404; 214/218/260/271/327/340/359/404; |
| | 214/218/260/271/304/327/340/359; 214/218/260/271/304/327/340/404; |
| | 214/218/260/271/304/327/340/411; 218/260/271/304/327/340/359/404; |
| | 218/260/271/304/327/340/359/411; 218/271/304/327/340/359/404/411; |
| | 218/260/304/327/340/359/404/411; 218/260/271/327/340/359/404/411; |
| | 218/260/271/304/340/359/404/411; 218/260/271/304/327/359/404/411; |
| | 218/260/271/304/327/340/404/411; 260/271/304/327/340/359/404/411; |
| | 214/271/304/327/340/359/404/411 |
| 9 | 214/218/260/271/304/327/340/359/404; 218/260/271/304/327/340/359/404/411; |
| | 214/260/271/304/327/340/359/404/411; 214/218/271/304/327/340/359/404/411; |
| | 214/218/260/304/327/340/359/404/411; 214/218/260/271/327/340/359/404/411; |
| | 214/218/260/271/304/340/359/404/411; 214/218/260/271/304/327/359/404/411; |
| | 214/218/260/271/304/327/340/404/411; 214/218/260/271/304/327/340/359/411 |
| 10 | 214/218/260/271/304/327/340/359/404/411 |

For example, the polypeptide may comprise a mutation corresponding to amino acid position 260 of SEQ ID NO: 1 or 2 or 3 or 4, wherein that amino acid is replaced with an alanine or a conservative variant of alanine, e.g., glycine, valine, leucine, or isoleucine. As another example, the polypeptide may comprise a mutation corresponding to amino acid position 214 of SEQ ID NO: 1 or 2 or 3 or 4, wherein that amino acid is replaced with glycine or a conservative variant of glycine, e.g., alanine, valine, leucine, or isoleucine. As yet another example, the polypeptide may comprise two mutations corresponding to amino acid positions 214 and 271 of SEQ ID NO: 1 or 2 or 3 or 4, wherein the amino acid at each position is replaced with the indicated amino acid or a conservative variant thereof.

In particular embodiments, the amino acid sequence of the mutant HPPD polypeptide of the invention is selected from the group consisting of SEQ ID NOs: 4-63 and 122-127.

In an embodiment, an isolated or recombinant polypeptide comprises an amino acid sequence encoding a 4-hydroxyphenylpyruvate dioxygenase (HPPD) protein that is tolerant to an HPPD inhibitor herbicide. In non-limiting embodiments, said mutant HPPD protein comprises an amino acid sequence having at least 50%, 60%, 65%, 75%, 80%, 85%, 90%, 91%, 92%, 93%, 94%, 95%, 96%, 97%, 98%, or 99% sequence identity to SEQ ID NO: 1, 2 or 3, wherein said amino acid sequence comprises a substitution at an amino acid position corresponding to amino acid position 214, position 271, or position 304 of SEQ ID NO: 1 or 2 or 3. In one example embodiment of said protein, the amino acid position corresponding to amino acid position 214 of SEQ ID NO: 1 or 2 or 3 is substituted with a glycine. In another example embodiment of said protein, the amino acid position corresponding to amino acid position 271 of SEQ ID NO: 1 or 2 or 3 is substituted with an asparagine. In another example embodiment of said protein, the amino acid position corresponding to amino acid position 304 of SEQ ID NO: 1 or 2 or 3 is substituted with a threonine.

In another example embodiment, said mutant HPPD protein has an amino acid sequence having at least 50%, 60%, 65%, 75%, 80%, 85%, 90%, 91%, 92%, 93%, 94%, 95%, 96%, 97%, 98%, or 99% sequence identity to SEQ ID NO: 1 or 2 or 3, and further comprises a substitution at one or more amino acid positions corresponding to amino acid positions 218, 260, 327, 340, 359, or 411 of SEQ ID NO: 1, 2 or 3. In one example embodiment of said protein, the amino acid position corresponding to amino acid position 218 of SEQ ID NO: 1 or 2 or 3 is substituted with an Isoleucine. In another example embodiment of said protein, the amino acid position corresponding to amino acid position 260 of SEQ ID NO: 1 or 2 or 3 is substituted with an Alanine. In another example embodiment of said protein, the amino acid position corresponding to amino acid position 327 of SEQ ID NO: 1 or 2 or 3 is substituted with an Arginine. In another example embodiment of said protein, the amino acid position corresponding to amino acid position 340 of SEQ ID NO: 1 or 2 or 3 is substituted with a Glutamic acid. In another example embodiment of said protein, the amino acid position corresponding to amino acid position 359 of SEQ ID NO: 1 or 2 or 3 or 2 or 3 is substituted with a methionine. In another example embodiment of said protein, the amino acid position corresponding to amino acid position 411 of SEQ ID NO: 1 is substituted with an Alanine. In another example embodiment of said protein, the amino acid position corresponding to amino acid position 404 of SEQ ID NO: 1 is substituted with an Asparagine.

In another example embodiment, said mutant HPPD protein has an amino acid sequence having at least 50%, 60%, 65%, 75%, 80%, 85%, 90%, 91%, 92%, 93%, 94%, 95%, 96%, 97%, 98%, or 99%, sequence identity to SEQ ID NO: 1 or 2 or 3, wherein said amino acid sequence comprises a substitution at an amino acid position corresponding to each of amino acid positions 218, 327, 340 and 359 of SEQ ID NO: 1 or 2 or 3. For example, the amino acid position corresponding to amino acid position 218 of SEQ ID NO:1 or 2 or 3 is substituted with an I, the amino acid position corresponding to amino acid position 327 of SEQ ID NO: 1 or 2 or 3 is substituted with an R, the amino acid position corresponding to amino acid position 340 of SEQ ID NO:1 or 2 or 3 is substituted with an E, and/or the amino acid position corresponding to amino acid position 359 is substituted with an M.

In another example embodiment, said mutant HPPD protein has an amino acid sequence having at least 50%, 60%, 65%, 75%, 80%, 85%, 90%, 91%, 92%, 93%, 94%, 95%, 96%, 97%, 98%, or 99% sequence identity to SEQ ID NO: 1, wherein said amino acid sequence comprises a substitution at an amino acid position corresponding to each of amino acid positions 218, 327, 340, 359 and G411 of SEQ ID NO: 1. For example, the amino acid position corresponding to amino acid position 218 of SEQ ID NO:1 is substituted with an I, the amino acid position corresponding to amino acid position 327 of SEQ ID NO: 1 is substituted with an R, the amino acid position corresponding to amino acid position 340 of SEQ ID NO:1 is substituted with an E, and the amino acid position corresponding to amino acid position 359 is substituted with an M, and the amino acid position corresponding to amino acid position 411 is substituted with an A.

In another example embodiment, said mutant HPPD protein has an amino acid sequence having at least 50%, 60%, 65%, 75%, 80%, 85%, 90%, 91%, 92%, 93%, 94%, 95%, 96%, 97%, 98%, or 99% sequence identity to SEQ ID NO: 1 or 2 or 3, wherein said amino acid sequence comprises a substitution at an amino acid position corresponding to each of amino acid positions 218, 260, 327, 340, 359 and 411 of SEQ ID NO: 1 or 2 or 3. For example, the amino acid position corresponding to amino acid position 218 of SEQ ID NO: 1 or 2 or 3 is substituted with an I, the amino acid position corresponding to amino acid position 260 is substituted with an A, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, the amino acid position corresponding to amino acid position 359 of SEQ ID NO: 1 or 2 or 3 is substituted with an M, and the amino acid position corresponding to amino acid position 411 of SEQ ID NO: 1 or 2 or 3 is substituted with an A.

In another example embodiment, said mutant HPPD protein has an amino acid sequence having at least 50%, 60%, 65%, 75%, 80%, 85%, 90%, 91%, 92%, 93%, 94%, 95%, 96%, 97%, 98%, or 99%, sequence identity to SEQ ID NO: 1 or 2 or 3, wherein said amino acid sequence comprises a substitution at an amino acid position corresponding to each of amino acid positions 218, 271, 327, 340 and 359 of SEQ ID NO: 1 or 2 or 3. For example, the amino acid position corresponding to amino acid position 218 of SEQ ID NO: 1 or 2 or 3 is substituted with an I, the amino acid position corresponding to amino acid position 271 of SEQ ID NO: 1 or 2 or 3 is substituted with an N, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, and the amino acid position corresponding to amino acid position 359 of SEQ ID NO: 1 or 2 or 3 is substituted with an M.

In another example embodiment, said mutant HPPD protein has an amino acid sequence having at least 50%, 60%, 65%, 75%, 80%, 85%, 90%, 91%, 92%, 93%, 94%, 95%, 96%, 97%, 98%, or 99% sequence identity to SEQ ID NO: 1 or 2 or 3, wherein said amino acid sequence comprises a substitution at an amino acid position corresponding to each of amino acid positions 214, 218, 327, 340, 359 and 411 of SEQ ID NO: 1 or 2 or 3. For example, the amino acid position corresponding to amino acid position 214 of SEQ ID NO: 1 or 2 or 3 is substituted with a G, the amino acid position corresponding to amino acid position 218 of SEQ ID NO: 1 or 2 or 3 is substituted with an I, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, the amino acid position corresponding to amino acid position 359 is substituted with a Y, and the amino acid position corresponding to amino acid position 411 is substituted with an A.

In another example embodiment, said mutant HPPD protein has an amino acid sequence having at least 50%, 60%, 65%, 75%, 80%, 85%, 90%, 91%, 92%, 93%, 94%, 95%, 96%, 97%, 98%, or 99% sequence identity to SEQ ID NO: 1 or 2 or 3, wherein said amino acid sequence comprises a substitution at an amino acid position corresponding to each of amino acid positions 214, 218, 304, 327, 340, 359 and 411 of SEQ ID NO: 1 or 2 or 3. For example, the amino acid position corresponding to amino acid position 214 of SEQ ID NO: 1 is substituted with a G, the amino acid position corresponding to amino acid 218 of SEQ ID NO: 1 or 2 or 3 is substituted with an I, the amino acid position corresponding to amino acid position 304 of SEQ ID NO: 1 or 2 or 3 is substituted with a T, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, and the amino acid position corresponding to amino acid position 359 of SEQ ID NO: 1 or 2 or 3 is substituted with a Y; and the amino acid position corresponding to amino acid position 411 of SEQ ID NO: 1 or 2 or 3 is substituted with an A.

In another example embodiment, said mutant HPPD protein has an amino acid sequence having at least 85%, 90%, 91%, 92%, 93%, 94%, 95%, 96%, 97%, 98%, or 99% sequence identity to any one of SEQ ID NOS: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 122, 123, 124, 125, 126, or 127. In yet another example embodiment, said mutant HPPD protein comprises the amino acid sequence of any one of SEQ ID NOS: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 122, 123, 124, 125, 126, or 127.

In further embodiments, the mutant HPPD protein of any of the above-mentioned embodiments further comprises a polypeptide motif comprising one or more amino acid substitutions or deletions corresponding to the motifs set forth in SEQ ID NO: 59, 60, 61, 62 or 63, wherein a position of the one or more amino acid substitutions of the motif are relative to corresponding one or more amino acids of SEQ ID NO: 1 or 2 or 3.

In further embodiments, an isolated or recombinant polynucleotide is provided encoding any of the herein-disclosed mutant HPPD proteins or polypeptides. As non-limiting embodiments, the isolated or recombinant polynucleotide encoding the mutant HPPD protein comprises nucleotide sequence having at least 85%, 90%, 91%, 92%, 93%, 94%, 95%, 96%, 97%, 98%, 99% or 100% identity to SEQ ID NO: 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 128, 129, 130, 131, 132, or 133. In embodiments of the isolated or recombinant polynucleotide, the nucleotide sequence of the isolated or recombinant polynucleotide is optimized for expression in a plant. In further embodiments of the isolated or recombinant polynucleotide, the polynucleotide is operably linked to a promoter. In example embodiments, the promoter drives expression in a plant or plant cell.

In embodiments, an expression cassette is provided comprising the herein disclosed isolated or recombinant polynucleotides encoding a mutant HPPD protein. In embodiments, the expression cassette further comprises, in addition to the isolated or recombinant polynucleotide encoding a mutant HPPD protein, another operably linked recombinant or isolated polynucleotide sequence encoding a polypeptide that confers a desirable trait. In example embodiments, the desirable trait is resistance to a herbicide, for example, the desirable trait is resistance to an HPPD inhibitor, glyphosate, a PPO inhibitor, or glufosinate or a Solanesyl Diphosphate Synthase (SDPS) inhibitor. In example embodiments, the polypeptide that confers a desirable trait is a cytochrome P450 or variant thereof. In other example embodiments, the polypeptide that confers a desirable trait is an EPSPS (5-enol-pyrovyl-shikimate-3-phosphate-synthase). In still other example embodiments, the polypeptide that confers a desirable trait is a Solanesyl Diphosphate Synthase (SDPS). In still other example embodiments, the polypeptide that confers a desirable trait is a phosphinothricin acetyl transferase (PAT) or a PPO. Non-limiting examples of mutant PPO polypeptides that can be used in combination with the mutant HPPD proteins provided herein include: US2015252379, U.S. Pat. Nos. 10,041,087, 10,087,460, 10,308,953, US2019161478 US2020270625, which disclose various mutant PPO proteins from Amaranthus and Alopecurus myosuroides and other organisms, each of which is herein incorporated by reference in its entirety. Additional examples of mutant PPO polypeptides that be used in combination with the mutant HPPD proteins provided herein include: U.S. Pat. No. 10,717,985, US2020277619, US2019330650, U.S. Pat. No. 10,844,395, US20210095305, WO2020251313, and WO2021133049, which disclose various mutant IPPO proteins from Cyanobacteria, each of which is herein incorporated by reference in its entirety. Non-limiting examples of mutant SPDS polypeptides that can be used in combination with the mutant HPPD proteins provided herein including US provisional application No. US62/850,248, filed 20 May 2019, entitled “Compositions and methods for weed control”, which disclose various mutant SPDS proteins, which is herein incorporated by reference in its entirety.

In embodiments, a vector is provided comprising an expression cassette comprising the isolated or recombinant polynucleotide encoding any of the herein disclosed mutant HPPD proteins. In example embodiments, the vector comprises one of SEQ ID NOs: 119, 120 or 121.

In embodiments, a cell is provided comprising a heterologous polynucleotide encoding any of the herein disclosed mutant HPPD polypeptides. In example embodiments, the cell is a plant cell. In embodiments, a plant or plant part is provided having stably integrated into its genome a heterologous polynucleotide encoding a mutant HPPD polypeptide. In example embodiments of the plant or plant part, an expression cassette comprising an isolated or recombinant polynucleotide encoding a mutant HPPD protein is stably incorporated into the genome of the plant. In example embodiments of the plant or plant part, the polynucleotide encoding said heterologous polypeptide has been introduced into the plant or plant part by transformation. In example embodiments of the plant or plant part, the polynucleotide encoding said heterologous polypeptide has been introduced into the genome of the plant or plant part by genome modification. In further embodiments, said recombinant polypeptide confers upon the plant increased herbicide tolerance as compared to the corresponding wild-type variety of the plant when expressed therein. In example embodiments of the plant or plant part, the plant is a monocot, for example, the plant is corn, rye, barley, rice, sorghum, oat, sorghum, sugarcane, switch grass, miscanthus grass, or wheat. In other example embodiments of the plant or plant part, the plant is a dicot, for example, the plant is soybean, sunflower, tomato, sugarbeet, tobacco, a cole crop, potato, sweet potato, cassava, safflower, trees, alfalfa, pea, and cotton.

In embodiments, the plant part comprises a seed that has stably incorporated into its genome a polynucleotide encoding a mutant HPPD polypeptide. In example embodiments, the seed is true breeding for an increased resistance to an HPPD inhibiting herbicide as compared to a wild-type variety of the seed.

In embodiments, a method is provided for conferring resistance to an HPPD inhibitor in a plant, the method comprising introducing an expression cassette comprising an isolated or recombinant polynucleotide encoding a herein-disclosed mutant HPPD protein into the plant or introducing a polynucleotide encoding a mutant HPPD polypeptide into the plant.

In further embodiments, a method of controlling undesired vegetation in an area of cultivation is provided wherein the method comprises the steps of: providing, at said area of cultivation, a plant having stably integrated into its genome a heterologous polynucleotide encoding a mutant HPPD polypeptide; and applying to said area of cultivation, an effective amount of an HPPD inhibitor herbicide or a composition comprising one or more additional herbicides. In example embodiments of the method, the plant comprises at least one additional heterologous nucleic acid comprising a nucleotide sequence encoding a herbicide tolerance enzyme. In example embodiments of the method, the HPPD inhibitor herbicide is applied simultaneously or sequentially with one or more additional herbicides. In example embodiments of the method, the HPPD inhibitor herbicide is selected from the group consisting of bicyclopyrone (CAS RN 352010-68-5), benzobicyclon (CAS RN 156963-66-5), benzofenap (CAS RN 82692-44-2), ketospiradox (CAS RN 192708-91-1) or its free acid (CAS RN 187270-87-7), isoxachlortole (CAS RN 141112-06-3), isoxaflutole (CAS RN 141112-29-0), mesotrione (CAS RN 104206-82-8), pyrasulfotole (CAS RN 365400-11-9), pyrazolynate (CAS RN 58011-68-0), pyrazoxyfen (CAS RN 71561-11-0), sulcotrione (CAS RN 99105-77-8), tefuryltrione (CAS RN 473278-76-1), tembotrione (CAS RN 335104-84-2), topramezone (CAS RN 210631-68-8), and agrochemically acceptable salts thereof. In an example embodiment, the HPPD inhibitor is mesotrione.

In example embodiments, a method of identifying or selecting a transformed plant cell, plant tissue, plant or part thereof is provided comprising the steps of: providing a transformed plant or plant part thereof, wherein said transformed plant or plant part comprises a polynucleotide encoding a mutant HPPD polypeptide operably linked to a promoter that drives expression in the plant or plant part; contacting the transformed plant or plant part with at least one HPPD inhibitor compound; determining whether the plant or plant part is affected by the HPPD inhibiting compound; and identifying or selecting the transformed plant or plant part having said polynucleotide. In another example embodiment, a method if provided for growing a plant transformed with a polynucleotide encoding a mutant HPPD polypeptide while controlling weeds in the vicinity of said plant, said method comprising the steps of: growing said plant; and applying an effective amount of a herbicide composition comprising an HPPD inhibitor to the plant and weeds.

In embodiments, a combination useful for weed control is provided comprising a polynucleotide encoding a mutant HPPD polypeptide as disclosed herein, which polynucleotide is capable of being expressed in a plant to thereby provides to that plant tolerance to an HPPD inhibiting herbicide; and an HPPD inhibiting herbicide. In embodiments, a process is provided for preparing a combination useful for weed control comprising: providing a polynucleotide encoding a mutant HPPD polypeptide as disclosed herein, which polynucleotide is capable of being expressed in a plant to thereby provide to that plant tolerance to an HPPD inhibiting herbicide; and (b) providing an HPPD inhibiting herbicide. In example embodiments of the process, said step of providing a polynucleotide comprises providing a plant containing the polynucleotide. In other example embodiments of the process, said step of providing a polynucleotide comprises providing a seed containing the polynucleotide. In example embodiments of the process, the plant is a monocot, for example, the plant is corn, rye, barley, rice, sorghum, oat, sorghum, sugarcane, switch grass, miscanthus grass, or wheat. In other example embodiments of the process, the plant is a dicot, for example, the plant is soybean, sunflower, tomato, sugarbeet, tobacco, a cole crop, potato, sweet potato, cassava, safflower, trees, alfalfa, pea, and cotton. In example embodiments of the process, the HPPD inhibiting herbicide is selected from the group consisting of bicyclopyrone (CAS RN 352010-68-5), benzobicyclon (CAS RN 156963-66-5), benzofenap (CAS RN 82692-44-2), ketospiradox (CAS RN 192708-91-1) or its free acid (CAS RN 187270-87-7), isoxachlortole (CAS RN 141112-06-3), isoxaflutole (CAS RN 141112-29-0), mesotrione (CAS RN 104206-82-8), pyrasulfotole (CAS RN 365400-11-9), pyrazolynate (CAS RN 58011-68-0), pyrazoxyfen (CAS RN 71561-11-0), sulcotrione (CAS RN 99105-77-8), tefuryltrione (CAS RN 473278-76-1), tembotrione (CAS RN 335104-84-2), topramezone (CAS RN 210631-68-8), and agrochemically acceptable salts thereof. In an example embodiment, the HPPD inhibiting herbicide is mesotrione. In further embodiments, the process further comprises a step of applying the HPPD inhibiting herbicide to the seed. Embodiments are also provided for use of the disclosed combination to control weeds at a plant cultivation site.

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the invention can be produced either from a nucleic acid disclosed herein, or by the use of standard molecular biology techniques. For example, a truncated protein of the invention can be produced by expression of a recombinant nucleic acid of the invention in an appropriate host cell, or alternatively by a combination of ex vivo procedures, such as protease digestion and purification. Accordingly, the present invention also provides nucleic acid molecules comprising polynucleotide sequences that encode mutant HPPD polypeptides that have HPPD enzymatic activity and that confer resistance or tolerance in plants to certain classes of herbicides that inhibit HPPD, and variants and fragments thereof. In general, the invention includes any polynucleotide sequence that encodes any of the mutant HPPD polypeptides described herein, as well as any polynucleotide sequence that encodes HPPD polypeptides having one or more conservative amino acid substitutions relative to the mutant HHPD polypeptides described herein. Conservative substitution tables providing functionally similar amino acids are well known in the art. The following five groups each contain amino acids that are conservative substitutions for one another: Aliphatic group: Glycine (G), Alanine (A), Valine (V), Leucine (L), Isoleucine (I); Aromatic group: Phenylalanine (F), Tyrosine (Y), Tryptophan (W); Sulfur-containing group: Methionine (M), Cysteine (C); Basic group: Arginine (R), Lysine (K), Histidine (H); Acidic group: Aspartic acid (D), Glutamic acid (E), Asparagine (N), Glutamine (Q).

In one embodiment, the present invention provides a polynucleotide sequence encoding an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to any of SEQ ID NOs: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 122, 123, 124, 125, 126, or 126 - where the HPPD amino acid sequence derives from a plant, where the polypeptide has HPPD enzymatic activity, and where the polypeptide contains one or more substitutions, additions or deletions as discussed infra.

In another embodiment, the present invention provides a polynucleotide sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to any of SEQ ID NOs SEQ ID NOs: 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 128, 129, 130, 131, 132, or 133.

As used herein, “nucleic acid” includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

As used herein, the terms “encoding” or “encoded” when used in the context of a specified nucleic acid mean that the nucleic acid comprises the requisite information to direct translation of the nucleotide sequence into a specified protein or amino acid sequence. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

As used herein, a “recombinant polynucleotide” comprises a polynucleotide that is not in its native or naturally occurring state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the polynucleotide sequence can be operably linked to a heterologous promoter which drives transcription of the polynucleotide sequence. As another example, the polynucleotide sequence can be cloned into a vector, or otherwise recombined with one or more additional nucleic acids that it is not combined with in nature. A “recombinant polypeptide” is a polypeptide produced by translation of a recombinant polynucleotide.

The invention encompasses isolated or purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. For example, the isolated polynucleotide or polypeptide is separated from other cellular components with which it is typically associated by any of various nucleic acid or protein purification methods. In specific embodiments, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of interfering enzyme activities and that is capable being characterized in respect of its catalytic, kinetic and molecular properties includes quite crude preparations of protein (for example recombinantly produced in cell extracts) having less than about 98%, 95% 90%, 80%, 70%, 60% or 50% (by dry weight) of contaminating protein as well as preparations further purified by methods known in the art to have 40%, 30%, 20%, 10%, 5%, or 1% (by dry weight) of contaminating protein. In embodiments, an “isolated polypeptide” is more enriched in (or out of) a cell than the polypeptide in its natural state in a wild-type cell, e.g., more than about 5% enriched, more than about 10% enriched, or more than about 20%, or more than about 50%, or more, enriched. Alternatively, this may be denoted as: 105%, 110%, 120%, 150% or more, enriched relative to wild type standardized at 100%. Such an enrichment is not the result of a natural response of a wild-type plant.

The proteins of the invention may be altered in various ways including amino acid substitutions, deletions, truncations, and insertions. Methods for such manipulations are generally known in the art. For example, amino acid sequence variants and fragments of the mutant HPPD proteins can be prepared by mutations in the DNA. Methods for mutagenesis and polynucleotide alterations are well known in the art. See, for example, Kunkel (1985) Proc. Natl. Acad. Sci. USA 82: 488-492; Kunkel et al. (1987) Methods in Enzymol. 154: 367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that often do not affect biological activity of the protein of interest may be found in the model of Dayhoff et al. (1978) Atlas of Protein Sequence and Structure (Natl. Biomed. Res. Found., Washington, D.C.). Conservative substitutions, such as exchanging one amino acid with another having similar properties, may be optimal.

The polynucleotides of the invention can also be used to isolate corresponding sequences from other organisms, particularly other plants. In this manner, methods such as PCR, hybridization, and the like can be used to identify such sequences based on their sequence homology to the sequences set forth herein.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art. See, for example, Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Methods for preparation of probes for hybridization and for construction of cDNA and genomic libraries are generally known in the art and are disclosed in Sambrook et al. (1989) Molecular Cloning: A Laboratory Manual (2^{nd} ed., Cold Spring Harbor Laboratory Press, Plainview, New York).

By “hybridizing to” or “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2× SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1× SSC at 45° C. for 15 minutes. An example low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2× SSC, 0.1% SDS at 50° C., more desirably in 7% sodium dodecyl sulfate (SDS), 0.5 M NaPO₄, 1 mM EDTA at 50° C. with washing in 1× SSC, 0.1% SDS at 50° C., more desirably still in 7% sodium dodecyl sulfate (SDS), 0.5 M NaPO₄, 1 mM EDTA at 50° C. with washing in 0.5× SSC, 0.1% SDS at 50° C., preferably in 7% sodium dodecyl sulfate (SDS), 0.5 M NaPO₄, 1 mM EDTA at 50° C. with washing in 0.1× SSC, 0.1% SDS at 50° C., more preferably in 7% sodium dodecyl sulfate (SDS), 0.5 M NaPO₄, 1 mM EDTA at 50° C. with washing in 0.1× SSC, 0.1% SDS at 65° C.

Fragments and variants of the disclosed nucleotide sequences and proteins encoded thereby are also encompassed by the present invention. “Fragment” is intended to mean a portion of the nucleotide sequence or a portion of the amino acid sequence and hence protein encoded thereby. Fragments of a nucleotide sequence may encode protein fragments that retain the biological activity of the mutant HPPD protein and hence have HPPD enzymatic activity. Alternatively, fragments of a nucleotide sequence that are useful as hybridization probes or in mutagenesis and shuffling reactions to generate yet further HPPD variants generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the polypeptides of the invention.

A fragment of a nucleotide sequence that encodes a biologically active portion of a mutant HPPD protein of the invention will encode at least 15, 25, 30, 40, 50, 60, 70, 80, 90, 100, 110, 120, 150, 180, 200, 250, 300, 350 contiguous amino acids, or up to the total number of amino acids present in a full-length mutant HPPD polypeptide provided herein, (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 122, 123, 124, 125, 126, or 126). Fragments of a nucleotide sequence that are useful as hybridization probes or PCR primers generally need not encode a biologically active portion of an HPPD protein.

As used herein, “full-length sequence” in reference to a specified polynucleotide means having the entire nucleic acid sequence of a native or mutated HPPD sequence. “Native sequence” is intended to mean an endogenous sequence, i.e., a non-engineered sequence found in an organism's genome.

Thus, a fragment of a nucleotide sequence of the invention may encode a biologically active portion of a mutant HPPD polypeptide, or it may be a fragment that can be used as a hybridization probe, etc., or PCR primer using methods disclosed below. A biologically active portion of a mutant HPPD polypeptide can be prepared by isolating a portion of one of the nucleotide sequences of the invention, expressing the encoded portion of the mutant HPPD protein (e.g., by recombinant expression in vitro), and assessing the activity of the encoded portion of the mutant HPPD protein. Nucleic acid molecules that are fragments of a nucleotide sequence of the invention comprise at least 15, 20, 50, 75, 100, 150, 200, 300, 400, 500, 600, 700, 800, 900, 1000, 1100, 1200, or 1300 contiguous nucleotides, or up to the number of nucleotides present in a full-length nucleotide sequence disclosed herein (i.e., any one of SEQ ID NOS: 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 128, 129, 130, 131, 132, or 133).

“Variants” is intended to mean substantially similar sequences. For polynucleotides, a variant comprises a deletion and/or addition of one or more nucleotides at one or more internal sites within the reference polynucleotide and/or a substitution of one or more nucleotides at one or more sites in the mutant HPPD polynucleotide. Similarly, for polypeptides, a variant comprises a deletion and/or addition of one or more amino acids at one or more internal sites within the reference polypeptide and/or a substitution of one or more amino acids at one or more sites in the mutant HPPD polypeptide. As used herein, a “reference” polynucleotide or polypeptide can comprise a mutant HPPD nucleotide sequence or amino acid sequence. Alternatively, the “reference” (or “control”) polynucleotide or polypeptide comprises a native polynucleotide or polypeptide. As a non-limiting example, a mutant HPPD polypeptide comprising amino acid substitutions at two sites may be used herein as a reference for a mutant HPPD polypeptide comprising amino acid substitutions at the same two sites and one or more additional sites. Alternatively, a first mutant HPPD polypeptide comprising amino acid substitutions at three sites may be used as a reference for a second, different mutant HPPD polypeptide comprising amino acid substitutions at three sites, wherein the three sites of the second polypeptide are partially overlapping or non-overlapping with the three sites of the first polypeptide. As used herein, a “native” polynucleotide or polypeptide comprises a naturally occurring nucleotide sequence or amino acid sequence, respectively. One of skill in the art will recognize that variants of the nucleic acids of the invention will be constructed such that the open reading frame is maintained. For polynucleotides, conservative variants include those sequences that, because of the degeneracy of the genetic code, encode the amino acid sequence of one of the mutant HPPD polypeptides of the invention. Naturally occurring allelic variants such as these can be identified with the use of well-known molecular biology techniques, such as, for example, with polymerase chain reaction (PCR) and hybridization techniques as outlined below. Variant polynucleotides also include synthetically derived polynucleotides, such as those generated, for example, by using site-directed mutagenesis but which still encode a mutant HPPD protein of the invention. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Mutant HPPD polypeptides of the invention can be generated by editing the endogenous HPPD gene in situ by way of genome modification techniques in order to provide a mutant HPPD polypeptide that is tolerant to an HPPD-inhibiting herbicide. Introduction may be accomplished by any manner known in the art, including and not limited to: introgression, transgenic, or through the use of a DNA modification enzyme.

Particularly, the modification to the nucleic acid sequence can be introduced by way of site-directed nucleases (SDN). More particularly, the SDN is selected from: meganuclease, zinc finger, transcription activator like effector nucleases system (TALEN) or Clustered Regularly Interspaced Short Palindromic Repeats system (CRISPR) system. SDN is also referred to as “genome editing”, or genome editing with engineered nucleases (GEEN). This is a type of genetic engineering in which DNA is inserted, deleted or replaced in the genome of an organism using engineered nucleases that create site-specific double-strand breaks (DSBs) at desired locations in the genome. The induced double-strand breaks are repaired through non-homologous end-joining (NHEJ) or homologous recombination (HR), resulting in targeted mutations ('edits'). Particularly SDN may comprises techniques such as: Meganucleases, Zinc finger nucleases (ZFNs), Transcription Activator-Like Effector-based Nucleases (TALEN) (Feng et al. 2013 Cell Res. 23, 1229-1232, Sander & Joung Nat. Biotechnol. 32, 347-355 2014), and the Clustered Regularly Interspaced Short Palindromic Repeats (CRISPR-Cas) system.

Accordingly, the current disclosure is also directed to vectors useful for editing. The vector includes a nucleic acid that encodes a DNA modification enzyme, such as a site-directed nuclease, e.g., a Cas9 nuclease, a Cfpl nuclease, a dCas9-FokI, a dCpfl-Fokl, a chimeric Cas9-cytidine deaminase, a chimeric Cas9-adenine deaminase, a chimeric FEN1-FokI, and a Mega-TALs, a nickase Cas9 (nCas9), a chimeric dCas9 non-Fokl nuclease and a dCpfl non-Fokl nuclease. The plasmid also includes at least one guide RNA. Vactors can also include additional components, for example, they may include a gRNA promoter, e.g., prOsU3-01, which is the Rice U3 promoter for pol III dependent transcription of non-coding RNAs, to regulate expression of the at least one gRNA. Vectors may similarly include additional features such as selectable markers, e.g Phosphomannose Isomerase (PMI) and can be used with mannose selection to recover stably transformed plants. Additional features include regulatory sequences, e.g., promoters and terminators for regulating expression of selectable markers.

Vectors may further include additional features to assist with transformation, e.g. features to assist with Agrobacterium-mediated transformation as described above. Target sequences may vary and may include a 15-25 nucleotide long sequence including a sequence, e.g., a 3 nucleotide sequence, that encodes an amino acid of Table 1 or Table 2.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptides of SEQ ID NOs: 4-63, and 122-128 are disclosed. Non-limiting examples of such polynucleotide sequences are disclosed at SEQ ID NOs: 64-118 and 128-133. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity across the entirety of the HPPD sequences described herein.

“Variant” protein is intended to mean a protein derived from the reference protein by deletion or addition of one or more amino acids at one or more internal sites in the mutant HPPD protein and/or substitution of one or more amino acids at one or more sites in the mutant HPPD protein. Variant proteins encompassed by the present invention are biologically active, that is they continue to possess the desired biological activity of the mutant HPPD protein, that is, HPPD enzymatic activity and/or herbicide tolerance as described herein. Such variants may result from, for example, genetic polymorphism or from human manipulation. Biologically active variants of a mutant HPPD protein of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity across the entirety of the amino acid sequence for the mutant HPPD protein as determined by sequence alignment programs and parameters described elsewhere herein. A biologically active variant of a protein of the invention may differ from that protein by as few as 1-15 amino acid residues, as few as 1-10 amino acid residues, such as 6-10 amino acid residues, as few as 5 amino acid residues, as few as 4, 3, 2, or even 1 amino acid residue.

Methods of alignment of sequences for comparison are well known in the art and can be accomplished using mathematical algorithms such as the algorithm of Myers and Miller (1988) CABIOS 4: 11-17; the local alignment algorithm of Smith et al. (1981) Adv. Appl. Math. 2: 482; the global alignment algorithm of Needleman and Wunsch (1970) J. Mol. Biol. 48: 443-453; and the algorithm of Karlin and Altschul (1990) Proc. Natl. Acad. Sci. USA 872264, modified as in Karlin and Altschul (1993) Proc. Natl. Acad. Sci. USA 90: 5873-5877. Computer implementations of these mathematical algorithms can be utilized for comparison of sequences to determine sequence identity. Such implementations include but are not limited to: CLUSTAL in the PC/Gene program (available from Intelligenetics, Mountain View, California); the ALIGN program (Version 2.0), GAP, BESTFIT, BLAST, FASTA, and TFASTA in the GCG Wisconsin Genetics Software Package, Version 10 (available from Accelrys Inc., 9685 Scranton Road, San Diego, California, USA), and BLOSUM62 in the Geneious Prime Software Package (available from Biomatters, Inc., 2365 Northside Dr. Suite 560, San Diego, CA 92108).

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48: 443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/ and as described in the following publication: “The EMBL-EBI search and sequence analysis tools APIs in 2019.” Madeira et al. Nucleic Acids Research, June 2019, 47(W1):W636-W641. The term “equivalent program” as used herein refers to any sequence comparison program t hat, for any two sequences in question, generates an alignment having identical nucleotide or amino acid residue matches and an identical percent sequence identity when compared to the corresponding alignment generated by EMBOSS Needle. In a preferred embodiment, substantially identical nucleic acid or amino acid sequences may perform substantially the same function.

Two nucleotide sequences can also be considered to be substantially identical when the two sequences hybridize to each other under stringent conditions. In representative embodiments, two nucleotide sequences considered to be substantially identical hybridize to each other under highly stringent conditions.

The terms “stringent conditions” or “stringent hybridization conditions” include reference to conditions under which a nucleic acid will selectively hybridize to a target sequence to a detectably greater degree than other sequences (e.g., at least 2-fold over a non-target sequence), and optionally may substantially exclude binding to non-target sequences. Stringent conditions are sequence-dependent and will vary under different circumstances. By controlling the stringency of the hybridization and/or washing conditions, target sequences can be identified that can be up to 100% complementary to the reference nucleotide sequence. Alternatively, conditions of moderate or even low stringency can be used to allow some mismatching in sequences so that lower degrees of sequence similarity are detected. For example, those skilled in the art will appreciate that to function as a primer or probe, a nucleic acid sequence only needs to be sufficiently complementary to the target sequence to substantially bind thereto so as to form a stable double-stranded structure under the conditions employed. Thus, primers or probes can be used under conditions of high, moderate or even low stringency Likewise, conditions of low or moderate stringency can be advantageous to detect homolog, ortholog and/or paralog sequences having lower degrees of sequence identity than would be identified under highly stringent conditions.

The terms “complementary” or “complementarity” (and similar terms), as used herein, refer to the natural binding of polynucleotides under permissive salt and temperature conditions by base-pairing. For example, the sequence “A-G-T” binds to the complementary sequence “T-C-A.” Complementarity between two single-stranded molecules may be partial, in which only some of the nucleotides bind, or it may be complete when total complementarity exists between the single stranded molecules. The degree of complementarity between nucleic acid strands has significant effects on the efficiency and strength of hybridization between the molecules. As used herein, the term “substantially complementary” (and similar terms) means that two nucleic acid sequences are at least about 50%, 60%, 70%, 75%, 80%, 85%, 90%, 95%, 96%, 97%, 98%, 99% or more complementary. Alternatively, the term “substantially complementary” (and similar terms) can mean that two nucleic acid sequences can hybridize together under high stringency conditions (as described herein).

As used herein, “specifically” or “selectively” hybridizing (and similar terms) refers to the binding, duplexing, or hybridizing of a molecule to a particular nucleic acid target sequence under stringent conditions when that sequence is present in a complex mixture (e.g., total cellular DNA or RNA) to the substantial exclusion of non-target nucleic acids, or even with no detectable binding, duplexing or hybridizing to non-target sequences. Specifically or selectively hybridizing sequences typically are at least about 40% complementary and are optionally substantially complementary or even completely complementary (i.e., 100% identical).

For DNA-DNA hybrids, the Tₘ can be approximated from the equation of Meinkoth and Wahl, Anal. Biochem., 138: 267-84 (1984): Tₘ=81.5° C.+16.6 (log M)+0.41 (% GC)-0.61 (% formamide)-500/L; where M is the molarity of monovalent cations, % GC is the percentage of guanosine and cytosine nucleotides in the DNA, % formamide is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired degree of identity. For example, if sequences with >90% identity are sought, the Tₘ can be decreased 10° C. Generally, stringent conditions are selected to be about 5° C. lower than the thermal melting point (Tₘfor the specific sequence and its complement at a defined ionic strength and pH. However, highly stringent conditions can utilize a hybridization and/or wash at the thermal melting point (Tₘ) or 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). If the desired degree of mismatching results in a Tₘ of less than 45° C. (aqueous solution) or 32° C. (formamide solution), optionally the SSC concentration can be increased so that a higher temperature can be used. An extensive guide to the hybridization of nucleic acids is found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier, New York (1993); Current Protocols in Molecular Biology, chapter 2, Ausubel, et al., eds, Greene Publishing and Wiley-Interscience, New York (1995); and Green & Sambrook, In: Molecular Cloning, A Laboratory Manual, 4th Edition, Cold Spring Harbor Press, Cold Spring Harbor, N.Y. (2012).

Typically, stringent conditions are those in which the salt concentration is less than about 1.5 M Na ion, typically about 0.01 to 1.0 M Na ion concentration (or other salts) at about pH 7.0 to pH 8.3 and the temperature is at least about 30° C. for short probes (e.g., 10 to 50 nucleotides) and at least about 60° C. for longer probes (e.g., greater than 50 nucleotides). Stringent conditions may also be achieved with the addition of destabilizing agents such as formamide or Denhardt's (5 g Ficoll, 5 g polyvinylpyrrolidone, 5 g bovine serum albumin in 500 ml of water). Exemplary low stringency conditions include hybridization with a buffer solution of 30% to 35% formamide, 1 M NaCl, 1% SDS (sodium dodecyl sulfate) at 37° C. and a wash in 1× to 2× SSC (20× SSC=3.0 M NaCl/0.3 M trisodium citrate) at 50° C. to 55° C. Exemplary moderate stringency conditions include hybridization in 40% to 45% formamide, 1 M NaCl, 1% SDS at 37° C. and a wash in 0.5× to 1× SSC at 55° C. to 60° C. Exemplary high stringency conditions include hybridization in 50% formamide, 1 M NaCl, 1% SDS at 37° C. and a wash in SSC at 60° C. to 65° C. A further non-limiting example of high stringency conditions include hybridization in 4× SSC, 5× Denhardt's, 0.1 mg/ml boiled salmon sperm DNA, and 25 mM Na phosphate at 65° C. and a wash in 0.1× SSC, 0.1% SDS at 65° C. Another illustration of high stringency hybridization conditions includes hybridization in 7% SDS, 0.5 M NaPO₄, 1 mM EDTA at 50° C. with washing in 2× SSC, 0.1% SDS at 50° C., alternatively with washing in 1× SSC, 0.1% SDS at 50° C., alternatively with washing in 0.5× SSC, 0.1% SDS at 50° C., or alternatively with washing in 0.1× SSC, 0.1% SDS at 50° C., or even with washing in 0.1× SSC, 0.1% SDS at 65° C. Those skilled in the art will appreciate that specificity is typically a function of post-hybridization washes, the relevant factors being the ionic strength and temperature of the final wash solution.

Nucleic acids that do not hybridize to each other under stringent conditions are still substantially identical if the proteins that they encode are substantially identical (e.g., due to the degeneracy of the genetic code).

A further indication that two nucleic acids or proteins are substantially identical is that the protein encoded by the first nucleic acid is immunologically cross reactive with the protein encoded by the second nucleic acid. Thus, a protein is typically substantially identical to a second protein, for example, where the two proteins differ only by conservative substitutions. Gene Stacking

In certain embodiments the polynucleotides of the invention encoding native or mutant HPPD polypeptides or variants thereof that retain HPPD enzymatic activity (e.g., a polynucleotide sequence encoding an amino acid sequence selected from the group consisting of SEQ ID NOs: 4-63; and 122-127 or a variant or fragment thereof) can be stacked with any combination of polynucleotide sequences of interest in order to create plants with a desired trait. A trait, as used herein, refers to the phenotype derived from a particular sequence or groups of sequences. For example, the polynucleotides encoding a mutant HPPD polypeptide, or variant thereof that retains HPPD enzymatic activity, may be stacked with any other polynucleotides encoding polypeptides that confer a desirable trait, including but not limited to resistance to diseases, insects, and herbicides, tolerance to heat and drought, reduced time to crop maturity, improved industrial processing, such as for the conversion of starch or biomass to fermentable sugars, and improved agronomic quality, such as high oil content and high protein content.

In a particular embodiment of the invention, polynucleotides may be stacked (or, alternatively, multiple expression cassettes may be stacked on a single polynucleotide) so as to express more than one type of HPPD polypeptide within a plant. This is a particular advantage where, for example, one HPPD is particularly suitable for providing resistance to one class of HPPD herbicide while the other provides improved tolerance to a different class of HPPD herbicide. Stacking HPPD polypeptides is also an advantage where one polypeptide expresses inherent herbicide-resistance but is somewhat labile. This herbicide-resistant HPPD can then be stabilised in mixed expression with, for example, similar but less temperature-labile HPPDs through the formation of mixed enzyme dimers.

Exemplary polynucleotides that may be stacked with polynucleotides of the invention encoding a mutant HPPD polypeptide or variant thereof that retains HPPD enzymatic activity include polynucleotides encoding polypeptides conferring resistance to pests/pathogens such as viruses, nematodes, insects or fungi, and the like. Exemplary polynucleotides that may be stacked with polynucleotides of the invention include polynucleotides encoding: polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48: 109), lectins (Van Damme et al. (1994) Plant Mol. Biol. 24: 825, pentin (described in U.S. Pat. No. 5,981,722), and the like; traits desirable for disease or herbicide resistance (e.g., fumonisin detoxification genes (U.S. Pat. No. 5,792,931); avirulence and disease resistance genes (Jones et al. (1994) Science 266: 789; Martin et al. (1993) Science 262: 1432; Mindrinos et al. (1994) Cell 78: 1089); a gene encoding an aryloxyalkanoate dioxygenase conferring resistance to certain classes of auxin and acetylCoA carboxylase herbicides (e.g. in PCT Publication Nos. WO 2008/141154, WO 2007/053482 or a tfdA gene giving resistance to 2,4 D in U.S. Pat. No. 6,153,401); a gene encoding a dicamba monoxygenase (Behrens et al. (2007) Science, 316, 1185) conferring resistance to dicamba; a gene encoding a homogentisate solanesyltransferase (HST) conferring resistance to HST-inhibiting herbicides (PCT Publication No. WO 2010/029311); a gene encoding a nitrilase conferring resistance to a nitrile-containing herbicide (e.g the bxnA bromoxynil nitrilase); acetolactate synthase (ALS) mutants that lead to herbicide resistance such as the S4 and/or Hra mutations; glyphosate resistance (e.g., 5-enol-pyrovyl-shikimate-3-phosphate-synthase (EPSPS) gene, described in U.S. Pat. Nos. 4,940,935 and 5,188,642; or the glyphosate N-acetyltransferase (GAT) gene, described in Castle et al. (2004) Science, 304: 1151-1154; and in U.S. Patent Application Publication Nos. 20070004912, 20050246798, and 20050060767)); glufosinate resistance (e.g, phosphinothricin acetyl transferase genes PAT and BAR, described in U.S. Pat. Nos. 5,561,236 and 5,276,268); a cytochrome P450 or variant thereof that confers herbicide resistance or tolerance to, inter alia, HPPD herbicides (U.S. Patent Application Publication No. 20090011936; U.S. Pat. Nos. 6,380,465; 6,121,512; 5,349,127; 6,649,814; and 6,300,544; and PCT Publication No. WO 2007/000077); and traits desirable for processing or process products such as high oil (e.g., U.S. Pat. No. 6,232,529); modified oils (e.g., fatty acid desaturase genes (U.S. Pat. No. 5,952,544; PCT Publication No. WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5.602,321; beta-ketothiolase, polyhydroxybutyrate synthase, and acetoacetyl-CoA reductase (Schubert et al. (1988) J. Bacteriol. 170: 5837-5847) facilitate expression of polyhydroxyalkanoates (PHAs)).

Thus, in one embodiment, the polynucleotides encoding a native or mutant HPPD polypeptide or variant thereof that retains HPPD enzymatic activity are stacked with one or more polynucleotides encoding polypeptides that confer resistance or tolerance to an herbicide. In one embodiment, the desirable trait is resistance or tolerance to an HPPD inhibitor. In another embodiment, the desirable trait is resistance or tolerance to glyphosate. In another embodiment, the desirable trait is resistance or tolerance to glufosinate. In further embodiments the desirable trait is resistance or tolerance to an HST inhibitor herbicide, an auxin herbicide or a PSII herbicide. In other embodiments, the desirable trait is resistance to glyphosate, a PPO inhibitor, or glufosinate or a Solanesyl Diphosphate Synthase inhibitor. In example embodiments, the polypeptide that confers a desirable trait is a cytochrome P450 or variant thereof. In other example embodiments, the polypeptide that confers a desirable trait is an EPSPS (5-enol-pyrovyl-shikimate-3-phosphate-synthase). In still other example embodiments, the polypeptide that confers a desirable trait is a phosphinothricin acetyl transferase (PAT) or a PPO. Non-limiting examples of mutant PPO polypeptides that can be used in combination with the mutant HPPD proteins provided herein include: US2015252379, U.S. Pat. Nos. 10,041,087, 10,087,460, 10,308,953, US2019161478, US2020270625, which disclose various mutant PPO proteins from Amaranthus and Alopecurus myosuroides and other organisms, each of which is herein incorporated by reference in its entirety. Additional examples of mutant PPO polypeptides that be used in combination with the mutant HPPD proteins provided herein include: U.S. Pat. No. 10,717,985, US2020277619, US2019330650, U.S. Pat. No. 10,844,395, US20210095305, WO2020251313, and WO2021133049, which disclose various mutant PPO proteins from Cyanobacteria, each of which is herein incorporated by reference in its entirety. In further example embodiments, the polypeptide that confers a desirable trait is an SDPS. Non-limiting examples of mutant SPDS polypeptides that can be used in combination with the mutant HPPD proteins provided herein including US provisional application No. U.S. Ser. No. 62/850,248, filed 20 May 2019, entitled “Compositions and methods for weed control”, which disclose various mutant SPDS proteins, which is herein incorporated by reference in its entirety.

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, PCT Publication Nos. WO 99/25821, WO 99/25854, WO 99/25840, WO 99/25855, and WO 99/25853.

### Plant Expression Cassettes

The compositions of the invention may additionally contain nucleic acid sequences for transformation and expression in a plant of interest. The nucleic acid sequences may be present in DNA constructs or expression cassettes. “Expression cassette” as used herein means a nucleic acid molecule capable of directing expression of a particular nucleotide sequence in an appropriate host cell, comprising a promoter operatively linked to the nucleotide sequence of interest (i.e., a polynucleotide encoding a mutant HPPD polypeptide or variant thereof that retains HPPD enzymatic activity, alone or in combination with one or more additional nucleic acid molecules encoding polypeptides that confer desirable traits) which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components (e.g., a promoter that is heterologous to the mutant HPPD polypeptide that it transcribes). The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. Additionally, the promoter can also be specific to a particular tissue or organ or stage of development.

The present invention encompasses the transformation of plants with expression cassettes capable of expressing a polynucleotide of interest to produce a polypeptide of interest, i.e., a polynucleotide encoding a mutant HPPD polypeptide or variant thereof that retains HPPD enzymatic activity, alone or in combination with one or more additional nucleic acid molecules encoding polypeptides that confer desirable traits. The expression cassette will include, in the 5′-3′ direction of transcription, a transcriptional and translational initiation region (i.e., a promoter) and a polynucleotide open reading frame encoding the polypeptide of interest. The expression cassette may optionally comprise a transcriptional and translational termination region (i.e., termination region) functional in plants. In some embodiments, the expression cassette comprises a selectable marker gene to allow for selection for stable transformants. In some embodiments, the expression cassette may include one or more additional regulatory elements to enhance expression of the polypeptide of interest, such as an enhancer, an intron, etc. Expression constructs of the invention may also comprise a leader sequence and/or a sequence allowing for inducible expression of the polynucleotide of interest. See, Guo et al. (2003) Plant J. 34: 383-92 and Chen et al. (2003) Plant J. 36: 731-40 for examples of sequences allowing for inducible expression.

The regulatory sequences of the expression construct are operably linked to the polynucleotide of interest. By “operably linked” is intended a functional linkage between a promoter and a second sequence wherein the promoter sequence initiates and mediates transcription of the DNA sequence corresponding to the second sequence. Generally, operably linked means that the nucleotide sequences being linked are contiguous.

Any promoter capable of driving expression in the plant of interest may be used in the practice of the invention. The promoter may be native or analogous or foreign or heterologous or exogenous to the plant host. The terms “heterologous” and “exogenous” when used herein to refer to a nucleic acid sequence (e.g. a DNA or RNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling, gene editing, mutagenesis, etc. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g., DNA) sequence is a nucleic acid (e.g., DNA or RNA) sequence naturally associated with a host cell into which it is introduced.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to that sequence. The promoters that are used for expression of the transgene(s) can be a strong plant promoter, a viral promoter, or a chimeric promoter composed of operably linked elements such as: TATA box from any gene (or synthetic, based on analysis of plant gene TATA boxes), optionally fused to the region 5′ to the TATA box of plant promoters (which direct tissue and temporally appropriate gene expression), optionally fused to 1 or more enhancers (such as the 35S enhancer, FMV enhancer, CMP enhancer, RUBISCO SMALL SUBUNIT enhancer, PLASTOCYANIN enhancer).

Exemplary constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313: 810-812); rice actin (McElroy et al. (1990) Plant Cell 2: 163-171); ubiquitin (Christensen et al. (1989) Plant Mol. Biol. 12: 619-632 and Christensen et al. (1992) Plant Mol. Biol. 18: 675-689); pEMU (Last et al. (1991) Theor. Appl. Genet. 81: 581-588); MAS (Velten et al. (1984) EMBO J. 3: 2723-2730); ALS promoter (U.S. Pat. No. 5,659,026), and the like. Other constitutive promoters are included in, for example, U.S. Pat. Nos. 5,608,149; 5,608,144; 5,604,121; 5,569,597; 5,466,785; 5,399,680; 5,268,463; 5,608,142; and 6,177,611.

Appropriate plant or chimeric promoters are useful for applications such as expression of transgenes in certain tissues, while minimizing expression in other tissues, such as seeds, or reproductive tissues. Exemplary cell type- or tissue-preferential promoters drive expression preferentially in the target tissue, but may also lead to some expression in other cell types or tissues as well. Methods for identifying and characterizing promoter regions in plant genomic DNA include, for example, those described in the following references: Jordano, et al., Plant Cell, 1: 855-866 (1989); Bustos, et al., Plant Cell, 1: 839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

In other embodiments of the present invention, inducible promoters may be desired. Inducible promoters drive transcription in response to external stimuli such as chemical agents or environmental stimuli. For example, inducible promoters can confer transcription in response to hormones such as giberellic acid or ethylene, or in response to light or drought.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV terminator, the tml terminator, the nopaline synthase terminator and the pea rbcs E9 terminator. These can be used in both monocotyledons and dicotyledons. In addition, a gene's native transcription terminator may be used.

Generally, the expression cassette will comprise a selectable marker gene for the selection of transformed cells. Selectable marker genes are utilized for the selection of transformed cells or tissues. In one example, the marker gene is phosphomannose isomerase (PMI) encoding for an enzyme that converts mannose-6-phosphate to fructose-6-phosphate. Only transformed cells having the marker gene are capable of utilizing mannose as a carbon source.

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize Adhl gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronze 1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from tobacco mosaic virus (TMV, the “W-sequence”), maize chlorotic mottle virus (MCMV), and alfalfa mosaic virus (AMV) have been shown to be effective in enhancing expression (e.g., Gallie et al. Nucl. Acids Res. 15: 8693-8711 (1987); Skuzeski et al. Plant Molec. Biol. 15: 65-79 (1990)). Other leader sequences known in the art include but are not limited to: picomavirus leaders, for example, EMCV leader (encephalomyocarditis 5′ noncoding region) (Elroy-Stein, O., Fuerst, T. R., and Moss, B. PNAS USA 86: 6126-6130 (1989)); potyvirus leaders, for example, tobacco etch virus (TEV) leader (Allison et al., 1986); maize dwarf mosaic virus (MDMV) leader; Virology 154: 9-20); human immunoglobulin heavy-chain binding protein (BiP) leader, (Macejak, D. G., and Samow, P., Nature 353: 90-94 (1991); untranslated leader from the coat protein mRNA of alfalfa mosaic virus (AMV RNA 4), (Jobling, S. A., and Gehrke, L., Nature 325: 622-625 (1987); tobacco mosaic virus leader (TMV), (Gallie, D. R. et al., Molecular Biology of RNA, 237-256 (1989); and maize chlorotic mottle virus leader (MCMV) (Lommel, S. A. et al., Virology 81: 382-385 (1991). See also, Della-Cioppa et al., Plant Physiology 84: 965-968 (1987).

The present invention also relates to nucleic acid constructs comprising one or more of the expression cassettes described above. The construct can be a vector, such as a plant transformation vector. In one embodiment, the vector is a plant transformation vector comprising a polynucleotide comprising the sequence set forth in SEQ ID NOs: 119-121.

### Plants

As used herein, the term “plant part” or “plant tissue” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. The aforementioned term also includes plant products, such as grain, fruits, and nuts.

Plants useful in the present invention include plants that are transgenic for at least a polynucleotide encoding a mutant HPPD polypeptide or variant thereof that retains HPPD enzymatic activity, alone or in combination with one or more additional nucleic acid molecules encoding polypeptides that confer desirable traits. The type of plant selected depends on a variety of factors, including for example, the downstream use of the harvested plant material, amenability of the plant species to transformation, and the conditions under which the plants will be grown, harvested, and/or processed. One of skill will further recognize that additional factors for selecting appropriate plant varieties for use in the present invention include high yield potential, good stalk strength, resistance to specific diseases, drought tolerance, rapid dry down and grain quality sufficient to allow storage and shipment to market with minimum loss.

Plants according to the present invention include any plant that is cultivated for the purpose of producing plant material that is sought after by man or animal for either oral consumption, or for utilization in an industrial, pharmaceutical, or commercial process. The invention may be applied to any of a variety of plants, including, but not limited to maize, wheat, rice, barley, soybean, cotton, sorghum, beans in general, rape/canola, alfalfa, flax, sunflower, safflower, millet, rye, sugarcane, sugar beet, cocoa, tea, Brassica, cotton, coffee, sweet potato, flax, peanut, clover; vegetables such as lettuce, tomato, cucurbits, cassava, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, and coconut; and flowers such as orchids, carnations and roses. Other plants useful in the practice of the invention include perennial grasses, such as switchgrass, prairie grasses, indiangrass, big bluestem grass and the like. It is recognized that mixtures of plants may be used.

The HPPD sequences and active variants and fragments thereof disclosed herein may be introduced into any plant species, including, but not limited to, monocots and dicots. Examples of plant species of interest include, but are not limited to, corn (Zea mays), Brassica sp. (e.g., B. napus, B. raga, B. juncea), particularly those Brassica species useful as sources of seed oil, alfalfa (Medicago sativa), rice (Oryza sativa), rye (Secale cereale), sorghum {Sorghum bicolor, Sorghum vulgare), millet (e.g., pearl millet (Pennisetum glaucum), proso millet (Panicum mitiaceum), foxtail millet (Setaria itatica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solatium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera, pineapple (Ananas comosus), citrus trees (Citrus spp.), cocoa (Theobroma cacao), tea (Camellia sinensis), banana (Musa spp.), avocado (Peryea americana), fig (Ficus carica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. meld), Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia puicherrima), and chrysanthemum.

Conifers that may he employed in practicing methods of the present disclosure include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis), and Poplar and Eucalyptus. In specific embodiments, plants of the present disclosure are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In addition, the term “crops” is to be understood as also including crops that have been rendered tolerant to herbicides or classes of herbicides (such as, for example, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors) as a result of conventional methods of breeding or genetic engineering. Examples of crops that have been rendered tolerant to herbicides or classes of herbicides by genetic engineering methods include glyphosate- and glufosinate-resistant crop varieties commercially available under the trade names RoundupReady® and LibertyLink®. The method according to the present invention is especially suitable for the protection of soybean crops which have also been rendered tolerant to any combination of glyphosate, dicamba, 2,4-D and/or glufosinate and where HPPD herbicides are used in a weed control programme along with other such herbicides (e.g glufosinate and/or glyphosate) for weed control.

It is further contemplated that the constructs of the invention may be introduced into plant varieties having improved properties suitable or optimal for a particular downstream use. For example, naturally-occurring genetic variability results in plants with resistance or tolerance to HPPD inhibitors or other herbicides, and such plants are also useful in the methods of the invention. The method according to the present invention can be further optimized by crossing the transgenes that provide a level of tolerance with soybean cultivars that exhibit an enhanced level of tolerance to HPPD inhibitors that is found in a small percentage of soybean lines.

Plant Transformation

Once an herbicide resistant or tolerant mutant HPPD polynucleotide, alone or in combination with one or more additional nucleic acid molecules encoding polypeptides that confer desirable traits, has been cloned into an expression system, it is introduced into a plant cell. The expression cassettes of the present invention can be introduced into the plant cell in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide is intended to mean that a polynucleotide is introduced into the plant and does not integrate into the genome of the plant.

“Stable transformation” or “stably transformed” is intended to mean that a polynucleotide, for example, a nucleotide construct described herein, introduced into a plant integrates into the genome of the plant and is capable of being inherited by the progeny thereof, more particularly, by the progeny of multiple successive generations.

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptll gene, which confers resistance to kanamycin and related antibiotics (Messing & Vierra Gene 19: 259-268 (1982); Bevan et al., Nature 304: 184-187 (1983)), the pat and bar genes, which confer resistance to the herbicide glufosinate (also called phosphinothricin; see White et al., Nucl. Acids Res 18: 1062 (1990), Spencer et al. Theor. Appl. Genet 79: 625-631 (1990) and U.S. Pat. Nos. 5,561,236 and 5,276,268), the hph gene, which confers resistance to the antibiotic hygromycin (Blochinger & Diggelmann, Mol. Cell Biol. 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), the glyphosate N-acetyltransferase (GAT) gene, which also confers resistance to glyphosate (Castle et al. (2004) Science, 304: 1151-1154; U.S. Patent App. Pub. Nos. 20070004912, 20050246798, and 20050060767); and the mannose-6-phosphate isomerase gene, which provides the ability to metabolize mannose (U.S. Pat. Nos. 5,767,378 and 5,994,629). Alternatively, and in one preferred embodiment the HPPD gene of the current invention is, in combination with the use of an HPPD herbicide as selection agent, itself used as the selectable marker.

Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens. These typically carry at least one T-DNA border sequence and include vectors such as pBIN19 (Bevan, Nucl. Acids Res. (1984)). For the construction of vectors useful in Agrobacterium transformation, see, for example, U.S. Patent Application Publication No. 2006/0260011.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g., PEG and electroporation) and microinjection. The choice of vector depends largely on the preferred selection for the species being transformed. For the construction of such vectors, see, e.g, U.S. Patent Application Publication No. 20060260011.

For expression of a nucleotide sequence of the present invention in plant plastids, plastid transformation vector pPH143 (see PCT Publication No. WO 97/32011, Example 36) is used. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g., pCIB200 or pCIB2001) to an appropriate Agrobacterium strain which may depend of the complement of vir genes carried by the host Agrobacterium strain either on a co-resident Ti plasmid or chromosomally (e.g., strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e., co-transformation) and both of these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

European patents EP 0 292 435 and EP 0 392 225, and PCT Publication No. WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, PCT Publication No. WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, PCT Publication No. WO 93/21335 describes techniques for the transformation of rice via electroporation.

European patent EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology 11: 1553-1558 (1993)) and Weeks et al. (Plant Physiol. 102:1077-1084 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSOG35) is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/l NAA, 5 mg/l GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described. See, PCT Publication No. WO 94/00977, U.S. Patent No. 5,591,616, and Negrotto et al., Plant Cell Reports 19: 798-803 (2000). For example, rice (Oryza sativa) can be used for generating transgenic plants. Various rice cultivars can be used (Hiei et al., 1994, Plant Journal 6: 271-282; Dong et al., 1996, Molecular Breeding 2: 267-276; Hiei et al., 1997, Plant Molecular Biology, 35: 205-218). Also, the various media constituents described below may be either varied in quantity or substituted. Embryogenic responses are initiated and/or cultures are established from mature embryos by culturing on MS-CIM medium (MS basal salts, 4.3 g/l; B5 vitamins (200×), 5 ml/l; sucrose, 30 g/l; proline, 500 mg/l; glutamine, 500 mg/l; casein hydrolysate, 300 mg/l; 2,4-D (1 mg/ml), 2 ml/l; adjust pH to 5.8 with 1 N KOH; phytagel, 3 g/l). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/l spectinomycin and any other appropriate antibiotic) for about 2 days at 28° C. Agrobacterium is re-suspended in liquid MS-CIM medium. The Agrobacterium culture is diluted to an OD⁶⁰⁰ of 0.2-0.3 and acetosyringone is added to a final concentration of 200 μM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with ticarcillin (400 mg/l) to inhibit the growth of Agrobacterium. For constructs utilizing the PMI selectable marker gene (Reed et al., In Vitro Cell. Dev. Biol.-Plant 37: 127-132), cultures are transferred to selection medium containing mannose as a carbohydrate source (MS with 2% mannose, 300 mg/l ticarcillin) after 7 days, and cultured for 3-4 weeks in the dark. Resistant colonies are then transferred to regeneration induction medium (MS with no 2,4-D, 0.5 mg/l IAA, 1 mg/l zeatin, 200 mg/l timentin 2% mannose and 3% sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% sorbitol) for 2 weeks and then moved to the greenhouse when they are large enough and have adequate roots. Plants are transplanted to soil in the greenhouse (To generation), grown to maturity, and the Ti seed is harvested.

The plants obtained via transformation with a nucleic acid sequence of interest in the present invention can be any of a wide variety of plant species, including those of monocots and dicots; however, the plants used in the method of the invention are preferably selected from the list of agronomically important target crops set forth elsewhere herein. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

For the transformation of plastids, seeds of Nicotiana tabacum c.v. “Xanthienc” are germinated seven per plate in a 1″ circular array on T agar medium and bombarded 12-14 days after sowing with 1 μm tungsten particles (M10, Biorad, Hercules, Calif.) coated with DNA from plasmids pPH143 and pPH145 essentially as described (Svab, Z. and Maliga, P. (1993) PNAS 90, 913-917). Bombarded seedlings are incubated on T medium for two days after which leaves are excised and placed abaxial side up in bright light (350-500 μmot photons/m²/s) on plates of RMOP medium (Svab, Z., Hajdukiewicz, P. and Maliga, P. (1990) PNAS 87, 8526-8530) containing 500 μg/ml spectinomycin dihydrochloride (Sigma, St. Louis, MO). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346349) is separated on 1% Tris-borate (TBE) agarose gels, transferred to nylon membranes (Amersham) and probed with ³²P-labeled random primed DNA sequences corresponding to a 0.7 kb BamHI/HindIII DNA fragment from pC8 containing a portion of the rps 7/12 plastid targeting sequence. Homoplasmic shoots are rooted aseptically on spectinomycin-containing MS/IBA medium (McBride, K. E. et al. (1994) PNAS 91, 7301-7305) and transferred to the greenhouse.

The genetic properties engineered into the transgenic seeds and plants described above are passed on by sexual reproduction or vegetative growth and can thus be maintained and propagated in progeny plants. Generally, maintenance and propagation make use of known agricultural methods developed to fit specific purposes such as tilling, sowing or harvesting.

Use of the advantageous genetic properties of the transgenic plants and seeds according to the invention can further be made in plant breeding. Depending on the desired properties, different breeding measures are taken. The relevant techniques are well known in the art and include but are not limited to hybridization, inbreeding, backcross breeding, multi-line breeding, variety blend, interspecific hybridization, aneuploid techniques, etc. Thus, the transgenic seeds and plants according to the invention can be used for the breeding of improved plant lines that, for example, increase the effectiveness of conventional methods such as herbicide or pesticide treatment or allow one to dispense with said methods due to their modified genetic properties.

The term “variety” as used herein refers to a group of plants within a species defined by the sharing of a common set of characteristics or traits accepted by those skilled in the art as sufficient to distinguish one cultivar or variety from another cultivar or variety. A cultivar or variety is considered “true breeding” for a particular trait if, when the true-breeding cultivar or variety is self-pollinated, all of the progeny contain the trait. The terms “breeding line” or “line” refer to a group of plants within a cultivar defined by the sharing of a common set of characteristics or traits accepted by those skilled in the art as sufficient to distinguish one breeding line or line from another breeding line or line. A breeding line or line is considered “true breeding” for a particular trait if, when the true-breeding line or breeding line is self-pollinated, all of the progeny contain the trait. As an example, an HPPD-resistant trait arises from a mutation in an HPPD gene of a plant or a seed of the plant.

Many suitable methods for transformation using suitable selection markers such as kanamycin, binary vectors such as from Agrobacterium and plant regeneration such as, for example, from tobacco leaf discs are well known in the art. Optionally, a control population of plants are likewise transformed with a polynucleotide expressing the control HPPD. Alternatively, an untransformed dicot plant such as Arabidopsis or tobacco can be used as a control since this, in any case, expresses its own endogenous HPPD.

### Herbicide Resistance

The present invention provides transgenic plants, plant cells, tissues, and seeds that have been transformed with a nucleic acid molecule encoding a mutant HPPD or variant thereof that confers resistance or tolerance to herbicides, alone or in combination with one or more additional nucleic acid molecules encoding polypeptides that confer desirable traits.

In one embodiment, the transgenic plants of the invention exhibit resistance or tolerance to application of herbicide in an amount of from about 5 to about 2,000 grams per hectare (g/ha), including, for example, about 5 g/ha, about 10 g/ha, about 15 g/ha, about 20 g/ha, about 25 g/ha, about 30 g/ha, about 35 g/ha, about 40 g/ha, about 45 g/ha, about 50 g/ha, about 55 g/ha, about 60 g/ha, about 65 g/ha, about 70 g/ha, about 75 g/ha, about 80 g/ha, about 85 g/ha, about 90 g/ha, about 95 g/ha, about 100 g/ha, about 110 g/ha, about 120 g/ha, about 130 g/ha, about 140 g/ha, about 150 g/ha, about 160 g/ha, about 170 g/ha, about 180 g/ha, about 190 g/ha, about 200 g/ha, about 210 g/ha, about 220 g/ha, about 230 g/ha, about 240 g/ha, about 250 g/ha, about 260 g/ha, about 270 g/ha, about 280 g/ha, about 290 g/ha, about 300 g/ha, about 310 g/ha, about 320 g/ha, about 330 g/ha, about 340 g/ha, about 350 g/ha, about360 g/ha, about 370 g/ha, about 380 g/ha, about 390 g/ha, about 400 g/ha, about 410 g/ha, about 420 g/ha, about 430 g/ha, about 440 g/ha, about 450 g/ha, about 460 g/ha, about 470 g/ha, about 480 g/ha, about 490 g/ha, about 500 g/ha, about 510 g/ha, about 520 g/ha, about 530 g/ha, about 540 g/ha, about 550 g/ha, about 560 g/ha, about 570 g/ha, about 580 g/ha, about 590 g/ha, about 600 g/ha, about 610 g/ha, about 620 g/ha, about 630 g/ha, about 640 g/ha, about 650 g/ha, about 660 g/ha, about 670 g/ha, about 680 g/ha, about 690 g/ha, about 700 g/ha, about 710 g/ha, about 720 g/ha, about 730 g/ha, about 740 g/ha, about 750 g/ha, about 760 g/ha, about 770 g/ha, about 780 g/ha, about 790 g/ha, about 800 g/ha, about 810 g/ha, about 820 g/ha, about 830 g/ha, about 840 g/ha, about 850 g/ha, about 860 g/ha, about 870 g/ha, about 880 g/ha, about 890 g/ha, about 900 g/ha, about 910 g/ha, about 920 g/ha, about 930 g/ha, about 940 g/ha, about 950 g/ha, about 960 g/ha, about 970 g/ha, about 980 g/ha, about 990 g/ha, about 1,000, g/ha, about 1,010 g/ha, about 1,020 g/ha, about 1,030 g/ha, about 1,040 g/ha, about 1,050 g/ha, about 1,060 g/ha, about 1,070 g/ha, about 1,080 g/ha, about 1,090 g/ha, about 1,100 g/ha, about 1,110 g/ha, about 1,120 g/ha, about 1,130 g/ha, about 1,140 g/ha, about 1,150 g/ha, about 1,160 g/ha, about 1,170 g/ha, about 1,180 g/ha, about 1,190 g/ha, about 1,200 g/ha, about 1,210 g/ha, about 1,220 g/ha, about 1,230 g/ha, about 1,240 g/ha, about 1,250 g/ha, about 1,260 g/ha, about 1,270 g/ha, about 1,280 g/ha, about 1,290 g/ha, about 1,300 g/ha, about 1,310 g/ha, about 1,320 g/ha, about 1,330 g/ha, about 1,340 g/ha, about 1,350 g/ha, about360 g/ha, about 1,370 g/ha, about 1,380 g/ha, about 1,390 g/ha, about 1,400 g/ha, about 1,410 g/ha, about 1,420 g/ha, about 1,430 g/ha, about 1,440 g/ha, about 1,450 g/ha, about 1,460 g/ha, about 1,470 g/ha, about 1,480 g/ha, about 1,490 g/ha, about 1,500 g/ha, about 1,510 g/ha, about 1,520 g/ha, about 1,530 g/ha, about 1,540 g/ha, about 1,550 g/ha, about 1,560 g/ha, about 1,570 g/ha, about 1,580 g/ha, about 1,590 g/ha, about 1,600 g/ha, about 1,610 g/ha, about 1,620 g/ha, about 1,630 g/ha, about 1,640 g/ha, about 1,650 g/ha, about 1,660 g/ha, about 1,670 g/ha, about 1,680 g/ha, about 1,690 g/ha, about 1,700 g/ha, about 1,710 g/ha, about 1,720 g/ha, about 1,730 g/ha, about 1,740 g/ha, about 1,750 g/ha, about 1,760 g/ha, about 1,770 g/ha, about 1,780 g/ha, about 1,790 g/ha, about 1,800 g/ha, about 1,810 g/ha, about 1,820 g/ha, about 1,830 g/ha, about 1,840 g/ha, about 1,850 g/ha, about 1,860 g/ha, about 1,870 g/ha, about 1,880 g/ha, about 1,890 g/ha, about 1,900 g/ha, about 1,910 g/ha, about 1,920 g/ha, about 1,930 g/ha, about 1,940 g/ha, about 1,950 g/ha, about 1,960 g/ha, about 1,970 g/ha, about 1,980 g/ha, about 1,990 g/ha, or about 2,000.

The average value and distribution of herbicide tolerance or resistance levels of a range of primary plant transformation events are evaluated in the normal manner based upon plant damage, meristematic bleaching symptoms, etc., at a range of different concentrations of herbicides. These data can be expressed in terms of, for example, GR₅₀ values derived from dose/response curves having “dose” plotted on the x-axis and “percentage kill”, “herbicidal effect”, “numbers of emerging green plants” etc. plotted on the y-axis where increased GR₅₀ values correspond to increased levels of inherent inhibitor-tolerance (e.g., increased k_{off}/K_{mHPP} value) and/or level of expression of the expressed HPPD polypeptide.

The methods of the present invention are especially useful to protect crops from the herbicidal injury of HPPD inhibitor herbicides. HPPD inhibitors are selected from the group consisting of bicyclopyrone (CAS RN 352010-68-5), bipyrazone (CAS RN 1622908-18-2), benquitrione (CAS RN 1639426-14-4), benzobicyclon (CAS RN 156963-66-5), benzofenap (CAS RN 82692-44-2), cypyrafluone (CAS RN 1855929-45-1), ketospiradox (CAS RN 192708-91-1) or its free acid (CAS RN 187270-87-7), dioxopyritrione (CAS RN 2222257-79-4 =Compound C), isoxachlortole (CAS RN 141112-06-3), fenquinotrione (CAS RN 1342891-70-6), fenpyrazone (CAS RN 1992017-55-6), isoxaflutole (CAS RN 141112-29-0), lancotrione (CAS RN 1486617-21-3), mesotrione (CAS RN 104206-82-8), pyrasulfotole (CAS RN 365400-11-9), pyrazolynate (CAS RN 58011-68-0), pyrazoxyfen (CAS RN 71561-11-0), sulcotrione (CAS RN 99105-77-8), tefuryltrione (CAS RN 473278-76-1), tembotrione (CAS RN 335104-84-2), tolpyralate (CAS RN 1101132-67-5), topramezone (CAS RN 210631-68-8), tripyrasulfone (CAS RN 1911613-97-2) and agrochemically acceptable salts thereof.

HPPD inhibitors further include compounds disclosed in WO2012/002096 (for example 6-(2,6-dioxocyclohexanecarbonyl)-4-(4-fluorophenyl)-2-methyl-1,2,4-triazine-3,5-dione), WO2012/126932 (for example 2-methyl-N-(5-methyl-1,3,4-oxadiazol-2-yl)-3-methylsulfonyl-4-(trifluoromethyl)benzamide), WO2012/028579 (for example 2-chloro-3-methylsulfanyl-N-(1-methyltetrazol-5-yl)-4-(trifluoromethyl)benzamide), WO2013/092834,

WO2013/139760, WO2013/144231, WO2014/192936, WO2015/128424, WO2016/038173, WO2016/135196, WO2018/050677 (for example N-(1-methyltetrazol-5-yl)-2-(1,2,4-triazol-1-yl)-6-(trifluoromethyl)pyridine-3-carboxamide=Compound D), WO2018/077875, WO2019/141740, WO2019/196904 (for example 3-(3-chlorophenyl)-6-(5-hydroxy-1,3-dimethyl-pyrazole-4-carbonyl)-1,5-dimethyl-quinazoline-2,4-dione and [4-[3-(3-chlorophenyl)-1,5-dimethyl-2,4-dioxo-quinazoline-6-carbonyl]-2,5-dimethyl-pyrazol-3-yl] N,N-diethylcarbamate), WO2019/243358, WO2020/108518 (for example 2-fluoro-N-(5-methyl-1,3,4-oxadiazol-2-yl)-3-[(R)-propylsulfinyl]-4-(trifluoromethyl) benzamide and 2-fluoro-N-(5-methyl-1,3,4-oxadiazol-2-yl)-3-propylsulfinyl-4-(trifluoromethyl) benzamide), WO2020/189576 (for example 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, WO2021/013969, WO2021/094505 and WO2021/209383.

### Methods of Use

The present invention further provides a method of selectively controlling weeds at a locus comprising crop plants and weeds, wherein the plants are obtained by any of the methods of the current invention described above, wherein the method comprises application to the locus of a weed controlling amount of one or more herbicides. Any of the transgenic plants described herein may be used within these methods of the invention. The term “locus” may include soil, seeds, seedlings, field, as well as established vegetation. Herbicides can suitably be applied pre-emergence or post-emergence of the crop or weeds.

The term “weed controlling amount” is meant to include, functionally, an amount of herbicide which is capable of affecting the growth or development of a given weed. Thus, the amount may be small enough to simply retard or suppress the growth or development of a given weed, or the amount may be large enough to irreversibly destroy a given weed. Further, the amount may be any amount there-between.

Thus, the present invention provides a method of controlling weeds at a locus comprising applying to the locus a weed-controlling amount of one or more herbicides, where the locus comprises a transgenic plant that has been transformed with a nucleic acid molecule encoding a mutant HPPD polypeptide or variant thereof that confers resistance or tolerance to HPPD herbicides, alone or in combination with one or more additional nucleic acid molecules encoding polypeptides that confer desirable traits. In one embodiment, the desirable trait is resistance or tolerance to an herbicide, including, for example, herbicides selected from the group consisting of an HPPD inhibitor, glyphosate, and glufosinate. In another embodiment, the locus comprises a transgenic plant that has been transformed with any combination of nucleic acid molecules described above, including one or more nucleic acid molecules encoding a mutant HPPD polypeptide or variant thereof that confers resistance or tolerance to an herbicide in combination with at least one, at least two, at least three, or at least four additional nucleic acid molecules encoding polypeptides that confer desirable traits.

In one embodiment, the present invention provides transgenic plants and methods useful for the control of unwanted plant species in crop fields, wherein the crop plants are made resistant to HPPD chemistry by transformation to express genes encoding mutant HPPD polypeptides, and where an HPPD herbicide is applied as an over-the-top application in amounts capable of killing or impairing the growth of unwanted vegetation or plant species (weed species, or, for example, carry-over or “rogue” or “volunteer” crop plants in a field of desirable crop plants). The application may be pre-or post emergence of the crop plants or of the unwanted species and may be combined with the application of other herbicides to which the crop is naturally tolerant, or to which it is resistant via expression of one or more other herbicide resistance transgenes. See, e.g., U.S. Patent Application Publication No. 2004/0058427 and PCT Publication No. WO 98/20144.

The one or more other herbicides that are applied in combination with the HPPD inhibiting herbicide can be applied sequentially (e.g., in succession) or simultaneously without affecting the yield or growth of the herbicide resistant plant. This includes applying the one or more other herbicides in accordance with a schedule based on the application of the HPPD inhibiting herbicide. When applied simultaneously, an effective amount of each herbicide is applied to the plant at the same time (e.g., at the same stage of plant growth, and/or as constituents of a herbicide composition comprising the one or more of herbicides and the HPPD inhibiting herbicide). As a non-limiting example, application of a herbicide composition comprising mesotrione and glufosinate to a plant comprising the mutant HPPD of the present disclosure is an example of simultaneous application. When applied sequentially, an effective amount of each herbicide is applied to the plant in succession. As a non-limiting example, application of mesotrione following application of glufosinate to a plant comprising the mutant HPPD of the present disclosure is an example of simultaneous application. In an alternate example of simultaneous application, mesotrione application occurs before application of glufosinate to a plant comprising the mutant HPPD of the present disclosure.

In another embodiment, the invention also relates to a method of protecting crop plants from herbicidal injury. In the cultivation of crop plants, especially on a commercial scale, correct crop rotation is crucially important for yield stability (the achievement of high yields of good quality over a long period) and for the economic success of an agronomic business. For example, across large areas of the main maize-growing regions of the USA (the “central corn belt”), soya is grown as the subsequent crop to maize in over 75% of cases. Selective weed control in maize crops is increasingly being carried out using HPPD inhibitor herbicides. Although that class of herbicides has excellent suitability for that purpose, it can result in agronomically unacceptable phytotoxic damage to the crop plants in subsequent crops (“carry-over” damage). For example, certain soya varieties are sensitive to even very small residues of such HPPD inhibitor herbicides. Accordingly, the herbicide resistant or tolerant plants of the invention are also useful for planting in a locus of any short term carry-over of herbicide from a previous application (e.g., by planting a transgenic plant of the invention in the year following application of an herbicide to reduce the risk of damage from soil residues of the herbicide).

Generally, the term “herbicide” is used herein to mean an active ingredient that kills, controls or otherwise adversely modifies the growth of plants. The “effective amount” or “effective concentration” of the herbicide is intended to mean an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest, such as weeds growing in the vicinity of the herbicide-resistant. Such an amount is known to those of ordinary skill in the art. Herbicidal activity is exhibited by herbicides useful for the present invention when they are applied directly to the plant or to the locus of the plant (such as at an area of cultivation where the plant is grown) at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted to promote non-selective or selective herbicidal action. In one example, the herbicide is applied postemergence to relatively immature undesirable vegetation to achieve the maximum control of weeds.

By “herbicide resistance” or “herbicide tolerance”, such as in the case of a “herbicide-resistant” or “herbicide-tolerant” plant, it is intended that a plant that is resistant to at least one herbicide at a level that would normally kill, or inhibit the growth of, a normal or wild-type plant. By mutant HPPD protein that is “herbicide-resistant” or “herbicide-tolerant”, it is intended that such an HPPD protein comprises one or more amino acid deletions, additions, or substitutions relative to a corresponding native or wild-type HPPD protein resulting in a higher HPPD activity relative to the HPPD activity of the corresponding native or wild-type HPPD protein, when in the presence of at least one herbicide that is known to interfere with HPPD activity and at a concentration or level of the herbicide that is known to inhibit the HPPD activity of a wild-type HPPD protein. Furthermore, the HPPD activity of such a herbicide-tolerant or herbicide-resistant mutated HPPD protein may be referred to herein as “herbicide-tolerant” or “herbicide-resistant” HPPD activity.

Non-limiting embodiments of the invention include:

1. An isolated or recombinant polypeptide comprising an amino acid sequence encoding a 4-hydroxyphenylpyruvate dioxygenase (HPPD) protein that is tolerant to an HPPD inhibitor herbicide compound, wherein said protein comprises:

(a) an amino acid sequence having at least 50%, 60%, 65%, 75%, 80%, 85%, 90%, or 95% sequence identity to SEQ ID NO: 1 or 2 or 3, wherein said amino acid sequence comprises a substitution at an amino acid position corresponding to amino acid positions 214, position 271 or position 304 of SEQ ID NO: 1 or 2 or 3;
(b) the amino acid sequence of (a), wherein the amino acid position corresponding to amino acid position 214 of SEQ ID NO: 1 or 2 or 3 is substituted with a G;
(c) the amino acid sequence of (a) wherein the amino acid position corresponding to amino acid position 271 of SEQ ID NO: 1 or 2 or 3 is substituted with an N;
(d) the amino acid sequence of (a) wherein the amino acid position corresponding to amino acid position 304 of SEQ ID NO: 1 or 2 or 3 is substituted with a T;
(e) the amino acid sequence of (a) further comprising a substitution at one or more amino acid position corresponding to amino acid position 218, 260, 327, 340, 359, or 411 of SEQ ID NO: 1 or 2 or 3;
(f) the amino acid sequence of (e), wherein the amino acid position corresponding to amino acid position 218 of SEQ ID NO: 1 or 2 or 3 is substituted with an I;
(g) the amino acid sequence of (e), wherein the amino acid position corresponding to amino acid position 260 of SEQ ID NO: 1 or 2 or 3 is substituted with an A;
(h) the amino acid sequence of (e), wherein the amino acid position corresponding to amino acid position 327 of SEQ ID NO: 1 or 2 or 3 is substituted with an R;
(i) the amino acid sequence of (e), wherein the amino acid position corresponding to amino acid position 340 of SEQ ID NO: 1 or 2 or 3 is substituted with an E;
(j) the amino acid sequence of (e), wherein the amino acid position corresponding to amino acid position 359 of SEQ ID NO: 1 or 2 or 3 is substituted with an M;
(k) the amino acid sequence of (e), wherein the amino acid position corresponding to amino acid position G411 of SEQ ID NO: 1 or 2 or 3 is substituted with an A;
(l) an amino acid sequence having at least 50%, 60%, 65%, 75%, 80%, 85%, 90%, or 95% sequence identity to SEQ ID NO: 1 or 2 or 3, wherein said amino acid sequence comprises a substitution at an amino acid position corresponding to each of amino acid positions 218, 327, 340 and 359 of SEQ ID NO: 1 or 2 or 3;
(m) an amino acid sequence of (1), wherein the amino acid position corresponding to amino acid position 218 of SEQ ID NO: 1 or 2 or 3 is substituted with an I, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, and the amino acid position corresponding to amino acid position 359 is substituted with an M;
(n) an amino acid sequence having at least 50%, 60%, 65%, 75%, 80%, 85%, 90%, or 95% sequence identity to SEQ ID NO: 1 or 2 or 3, wherein said amino acid sequence comprises a substitution at an amino acid position corresponding to each of amino acid positions 218, 327, 340, 359 and G411 of SEQ ID NO: 1 or 2 or 3;
(m) an amino acid sequence of (n), wherein the amino acid position corresponding to amino acid position 218 of SEQ ID NO: 1 or 2 or 3 is substituted with an I, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, and the amino acid position corresponding to amino acid position 359 is substituted with an M, and the amino acid position corresponding to amino acid position 411 is substituted with an A;
(o) an amino acid sequence having at least 50%, 60%, 65%, 75%, 80%, 85%, 90%, or 95% sequence identity to SEQ ID NO: 1 or 2 or 3, wherein said amino acid sequence comprises a substitution at an amino acid position corresponding to each of amino acid positions 218, 260, 327, 340, 359 and 411 of SEQ ID NO: 1 or 2 or 3;
(p) an amino acid sequence of (o), wherein the amino acid position corresponding to amino acid position 218 of SEQ ID NO: 1 or 2 or 3 is substituted with an I, the amino acid position corresponding to amino acid position 260 of SEQ ID NO: 1 or 2 or 3 is substituted with an A, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, the amino acid position corresponding to amino acid position 359 of SEQ ID NO: 1 or 2 or 3 is substituted with an M, and the amino acid position corresponding to amino acid position 411 of SEQ ID NO: 1 or 2 or 3 is substituted with an A;
(q) an amino acid sequence having at least 50%, 60%, 65%, 75%, 80%, 85%, 90%, or 95% sequence identity to SEQ ID NO: 1 or 2 or 3, wherein said amino acid sequence comprises a substitution at an amino acid position corresponding to each of amino acid positions 218, 271, 327, 340 and 359 of SEQ ID NO: 1;
(r) an amino acid sequence of (q), wherein the amino acid position corresponding to amino acid position 218 of SEQ ID NO: 1 or 2 or 3 is substituted with an I, the amino acid position corresponding to amino acid position 271 of SEQ ID NO: 1 or 2 or 3 is substituted with an N, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, and the amino acid position corresponding to amino acid position 359 of SEQ ID NO: 1 or 2 or 3 is substituted with an M;
(s) an amino acid sequence having at least 50%, 60%, 65%, 75%, 80%, 85%, 90%, or 95% sequence identity to SEQ ID NO: 1 or 2 or 3, wherein said amino acid sequence comprises a substitution at an amino acid position corresponding to each of amino acid positions 214, 218, 327, 340, 359 and 411 of SEQ ID NO: 1 or 2 or 3;
(t) an amino acid sequence of (s), wherein the amino acid position corresponding to amino acid position 214 of SEQ ID NO: 1 or 2 or 3 is substituted with a G, the amino acid position corresponding to amino acid position 218 of SEQ ID NO: 1 or 2 or 3 is substituted with an I, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, the amino acid position corresponding to amino acid position 359 is substituted with a Y, and the amino acid position corresponding to amino acid position 411 is substituted with an A;
(u) an amino acid sequence having at least 50%, 60%, 65%, 75%, 80%, 85%, 90%, or 95% sequence identity to SEQ ID NO: 1 or 2 or 3, wherein said amino acid sequence comprises a substitution at an amino acid position corresponding to each of amino acid positions 214, 218, 304, 327, 340, 359 and 411 of SEQ ID NO: 1 or 2 or 3;
(v) an amino acid sequence of (u), wherein the amino acid position corresponding to amino acid position 214 of SEQ ID NO: 1 is substituted with a G, the amino acid position corresponding to amino acid 218 of SEQ ID NO: 1 or 2 or 3 is substituted with an I, the amino acid position corresponding to amino acid position 304 of SEQ ID NO: 1 or 2 or 3 is substituted with a T, the amino acid position corresponding to amino acid position 327 of SEQ ID NO: 1 or 2 or 3 is substituted with an R, the amino acid position corresponding to amino acid position 340 of SEQ ID NO: 1 or 2 or 3 is substituted with an E, and the amino acid position corresponding to amino acid position 359 is substituted with a Y; and the amino acid position corresponding to amino acid position 411 of SEQ ID NO: 1 or 2 or 3 is substituted with an A;
(w) an amino acid sequence having at least 85%, 90%, 95%, or 98% sequence identity to any one of SEQ ID NOS: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 122, 123, 124, 125, 126, or 127;
(x) an amino acid sequence set forth any one of SEQ ID NOS: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 122, 123, 124, 125, 126, or 127;
(y) an amino acid sequence of (a)-(w), further comprising a polypeptide motif comprising one or more amino acid substitutions or deletions corresponding to the motifs set forth in SEQ ID NO: 59, 60, 61, 62 or 63 and wherein a position of the one or more amino acid substitutions of the motif are relative to corresponding one or more amino acids of SEQ ID NO: 1 or 2 or 3.
2. An isolated or recombinant polynucleotide encoding the polypeptide of embodiment 1.
3. The isolated or recombinant polynucleotide of embodiment 2, wherein the polynucleotide comprises SEQ ID NO: 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 128, 129, 130, 131, 132, or 133.
4. The isolated or recombinant polynucleotide of embodiment 2 or 3, wherein the nucleotide sequence of the isolated polynucleotide is optimized for expression in a plant.
5. The isolated or recombinant polynucleotide of embodiment 2, 3, or 4, wherein said polynucleotide is operably linked to a promoter.
6. The isolated or recombinant polynucleotide of embodiment 5, wherein the promoter drives expression in a plant or plant cell.
7. An expression cassette comprising the isolated polynucleotide of embodiment 2, 3, 4, 5 or 6.
8. The expression cassette of embodiment 7, further comprising an operably linked recombinant or isolated polynucleotide sequence encoding a polypeptide that confers a desirable trait.
9. The expression cassette of embodiment 8, wherein the desirable trait is resistance to an herbicide.
10. The expression cassette of embodiment 9, wherein said desirable trait is resistance to an HPPD inhibitor, glyphosate, PPO inhibitor, or glufosinate.
11. The expression cassette of embodiment 10, wherein said polypeptide that confers a desirable trait is a cytochrome P450 or variant thereof.
12. The expression cassette of embodiment 10, wherein said polypeptide that confers a desirable trait is an EPSPS (5-enol-pyrovyl-shikimate-3-phosphate-synthase).
13. The expression cassette of embodiment 10, wherein said polypeptide that confers a desirable trait is a phosphinothricin acetyl transferase (PAT) or a PPO.
14. A vector comprising the expression cassette of embodiment 7, 8, or 9.
15. The vector of embodiment 14, wherein the vector comprises one of SEQ ID NOs: 119, 120 or 121.
16. A cell comprising a heterologous polynucleotide encoding the polypeptide of claim embodiment.
17. The cell of embodiment 16, wherein said cell is a plant cell.
18. A plant or plant part having stably integrated into its genome a heterologous polynucleotide encoding the polypeptide of embodiment 1.
19. The plant or plant part of embodiment 18, wherein said plant has stably incorporated into its genome the expression cassette of any one of embodiments 7-13.
20. The plant or plant part of embodiment 18, wherein said polynucleotide encoding said heterologous polypeptide has been introduced into the plant or plant part by transformation.
21. The plant or plant part of embodiment 18, wherein said polynucleotide encoding said heterologous polypeptide has been introduced into the genome by genome modification.
22. The plant or plant part of embodiment 18, 19, 20 or 21, wherein said recombinant polypeptide confers upon the plant increased herbicide tolerance as compared to the corresponding wild-type variety of the plant when expressed therein.
23. The plant or plant part of embodiments 18, 19, 20, 21 or 22, wherein said plant is a monocot.
24. The plant or plant part of embodiment 23, wherein said monocot is corn, rye, barley, rice, sorghum, oat, sorghum, sugarcane, switch grass, miscanthus grass, or wheat
25. The plant or plant part of embodiment 18, 19, 20, 21, or 22, wherein said plant is a dicot.
26. The plant or plant part of embodiment 25, wherein said dicot is soybean, sunflower, tomato, sugarbeet, tobacco, a cole crop, potato, sweet potato, cassava, safflower, trees, alfalfa, pea, and cotton.
27. A seed produced by the plant of any one of embodiments 18-26, wherein said seed has stably incorporated into its genome a polynucleotide encoding the polypeptide of embodiment 1.
28. A seed of claim 27, wherein the seed is true breeding for an increased resistance to an HPPD inhibiting herbicide as compared to a wild-type variety of the seed.
29. A method for conferring resistance to an HPPD inhibitor in a plant, the method comprising introducing the expression cassette of any one of embodiments 7-13 into the plant or introducing a polynucleotide encoding a polypeptide of claim 1 into the plant.
30. A method of controlling undesired vegetation in an area of cultivation, the method comprising
a) providing, at said area of cultivation, a plant of any one of embodiments 18-26,
b) applying to said area of cultivation, an effective amount of an HPPD inhibitor compound.
31. The method of embodiment 30, wherein the plant comprises at least one additional heterologous nucleic acid comprising a nucleotide sequence encoding a herbicide tolerance enzyme.
32. The method of embodiment 30 or 31, wherein the HPPD inhibitor herbicide is applied simultaneously or sequentially with one or more additional herbicide.
33. The method of any one of embodiments 29-32, or the compositions of any one of embodiments 1-28, wherein the one or more HPPD inhibitors are selected from the group consisting of bicyclopyrone (CAS RN 352010-68-5), benzobicyclon (CAS RN 156963-66-5), benzofenap (CAS RN 82692-44-2), ketospiradox (CAS RN 192708-91-1) or its free acid (CAS RN 187270-87-7), isoxachlortole (CAS RN 141112-06-3), isoxaflutole (CAS RN 141112-29-0), mesotrione (CAS RN 104206-82-8), pyrasulfotole (CAS RN 365400-11-9), pyrazolynate (CAS RN 58011-68-0), pyrazoxyfen (CAS RN 71561-11-0), sulcotrione (CAS RN 99105-77-8), tefuryltrione (CAS RN 473278-76-1), tembotrione (CAS RN 335104-84-2), topramezone (CAS RN 210631-68-8), and agrochemically acceptable salts thereof.
34. The method of any one of embodiments 29-32 or the composition of any one of embodiments 1-28, wherein the one or more HPPD inhibitors is mesotrione.
35. A method of identifying or selecting a transformed plant cell, plant tissue, plant or part thereof comprising:
i) providing a transformed plant or plant part thereof, wherein said transformed plant or plant part comprises a polynucleotide encoding a polypeptide of claim 1 operably linked to a promoter that drives expression the plant or plant part;
ii) contacting the transformed plant or plant part with at least one HPPD inhibitor compound;
iii) determining whether the plant or plant part is affected by the HPPD inhibiting compound; and
iv) identifying or selecting the transformed plant or plant part having said polynucleotide.
35. A method for growing a plant of any one of embodiments 18-26 while controlling weeds in the vicinity of said plant, said method comprising the steps of:
a) growing said plant; and
b) applying an effective amount of a herbicide composition comprising an HPPD inhibitor to the plant and weeds.
36. A combination useful for weed control, comprising
(a) a polynucleotide encoding a polypeptide of embodiment 1, which polynucleotide is capable of being expressed in a plant to thereby provides to that plant tolerance to an HPPD inhibiting herbicide; and
(b) an HPPD inhibiting herbicide.
37. A process for preparing a combination useful for weed control comprising
(a) providing a polynucleotide encoding a HPPD polypeptide of embodiment 1, which polynucleotide is capable of being expressed in a plant to thereby provide to that plant tolerance to an HPPD inhibiting herbicide; and
(b) providing an HPPD inhibiting herbicide.
38. The process according to embodiment 37, wherein said step of providing a polynucleotide comprises providing a plant containing the polynucleotide.
39. The process according to embodiment 37, wherein said step of providing a polynucleotide comprises providing a seed containing the polynucleotide.
40. The process according to claim 39, further comprising a step of applying the HPPD inhibiting herbicide to the seed.
41. Use of a combination of embodiments 26 to control weeds at a plant cultivation site.
42. The method of claim 35 or the process according to any of claims 37-40 wherein the plant is a monocot, optionally wherein the monocot is corn, rye, barley, rice, sorghum, oat, sorghum, sugarcane, switch grass, miscanthus grass, or wheat
43. The method of claim 35 or the process according to any of claims 37-40 wherein the plant is a dicot, optionally wherein the dicot is soybean, sunflower, tomato, sugarbeet, tobacco, a cole crop, potato, sweet potato, cassava, safflower, trees, alfalfa, pea, and cotton.
44. The method of claim 35 or the process according to any of claims 37-43, wherein the one or more HPPD inhibitors are selected from the group consisting of bicyclopyrone, benzobicyclon, benzofenap, ketospiradox or its free acid, isoxachlortole, isoxaflutole, mesotrione, pyrasulfotole, pyrazolynate, pyrazoxyfen, sulcotrione, tefuryltrione, tembotrione, topramezone, and agrochemically acceptable salts thereof.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1: Cloning, Expression and Assay of Avena, Alopecurus and Apera-Derived HPPD Sequences and Determination of IC50 Values of the Resulting HPPD Mutants Versus Various HPPD Herbicides

DNA sequences (SEQ ID NOs: 65-118, 128, 130, 131, and 133), synthesized for expression in E. coli, by GeneWiz (USA) to encode HPPD variants (SEQ ID NOs: 5-58, 122, 124, 125 and 127) derived from either Avena sativa (SEQ ID NOs: 122, 124), Alopecurus myosuroides (SEQ ID NO: 126) or Apera spica-venti (SEQ ID NOs: 123, 125) were transformed into E coli BL21 (DE3) (New England Biolabs) and grown in 0.5 ml Terrific broth +50 ug/ml Kanamycin media in a 96-deepwell block overnight (37° C., 900rpm). 25 ul of the overnight cultures were inoculated into fresh 96-deepwell block containing 1 ml Formedium Autoinduction Media (AIM). The AIM cultures were grown for 3h at 37° C. (900rpm) and then transferred to 20° C. overnight (900rpm) to allow protein expression. The overnight cultures were centrifuged and 500 ul of Cellytic B (Sigma Aldrich) was added to resuspend the cell pellets. The coupled HGO assay (Siehl et al, 2014) is used to generate IC50 values for the HPPD variants of interest.

E. coli BL21 (DE3) cells were transformed with a pET24a vector containing a C-terminally his-tagged Arabidopsis HGO gene, grown and induced overnight as described for the HPPD variants above. The cell pellet was resuspended in phosphate buffered saline at pH 7.4 containing 10% Glycerol, 1mm Tris[2-carboxyethyl] phosphine-HCl (TCEP) plus 30 mM Imidazole and lysed using lysozyme/benzonase. The extract was clarified by low speed centrifugation and purified in the same buffer down a 1 ml His Gravitrap column. The bound HGO was eluted in 0.25M imidazole and the fractions pooled and beaded into liquid nitrogen. All procedures apart from the centrifugation and beading steps were carried out under a nitrogen atmosphere.

The coupled HGO assay is carried out by adding 10 ul of HPPD extract to a 96-well plate and then adding 40 ul of reaction buffer (50 mM Bis Tris Propane, pH7.0, 25 mM sodium-L-ascorbate, 400μM mercaptoethanol, 50 μM Fe(II)SO₄). The plates are left to incubate for 5 minutes on ice. 1 ul of the HPPD inhibitor of interest is added to the wells at a range of concentrations to allow an IC50 value to be calculated (such as 0.1-500 uM). 200 ul of HPP/HGO (197 μM hydroxyphenylpyruvic acid, 16 μl/ml Homogentisic Acid Oxidase in reaction buffer as described above) buffer is then added to the well and the reaction is regularly monitored (e.g. every 30 seconds-5 minutes) for absorbance at 330 nm for a given time period (e.g., for between 15-60 minutes). The data can then be used to calculate the IC50 value for each HPPD variant of interest.

HPPD inhibitors of interest tested were selected from the group consisting of bicyclopyrone (CAS RN 352010-68-5), bipyrazone (CAS RN 1622908-18-2), benquitrione (CAS RN 1639426-14-4), benzobicyclon (CAS RN 156963-66-5), benzofenap (CAS RN 82692-44-2), cypyrafluone (CAS RN 1855929-45-1), ketospiradox (CAS RN 192708-91-1) or its free acid (CAS RN 187270-87-7), dioxopyritrione (CAS RN 2222257-79-4, herein referred to as Herbicide C), isoxachlortole (CAS RN 141112-06-3), fenquinotrione (CAS RN 1342891-70-6), fenpyrazone (CAS RN 1992017-55-6), isoxaflutole (CAS RN 141112-29-0), lancotrione (CAS RN 1486617-21-3), mesotrione (CAS RN 104206-82-8), pyrasulfotole (CAS RN 365400-11-9), pyrazolynate (CAS RN 58011-68-0), pyrazoxyfen (CAS RN 71561-11-0), sulcotrione (CAS RN 99105-77-8), tefuryltrione (CAS RN 473278-76-1), tembotrione (CAS RN 335104-84-2), tolpyralate (CAS RN 1101132-67-5), topramezone (CAS RN 210631-68-8), tripyrasulfone (CAS RN 1911613-97-2) and agrochemically acceptable salts thereof.

HPPD inhibitors further include compounds disclosed in WO2012/002096 (for example 6-(2,6-dioxocyclohexanecarbonyl)-4-(4-fluorophenyl)-2-methyl-1,2,4-triazine-3,5-dione), WO2012/126932 (for example 2-methyl-N-(5-methyl-1,3,4-oxadiazol-2-yl)-3-methylsulfonyl-4-(trifluoromethyl)benzamide), WO2012/028579 (for example 2-chloro-3-methylsulfanyl-N-(1-methyltetrazol-5-yl)-4-(trifluoromethyl)benzamide), WO2013/092834, WO2013/139760, WO2013/144231, WO2014/192936, WO2015/128424, WO2016/038173, WO2016/135196, WO2018/050677 (for example N-(1-methyltetrazol-5-yl)-2-(1,2,4-triazol-1-yl)-6-(trifluoromethyl)pyridine-3-carboxamide, herein referred to as Herbicide D), WO2018/077875, WO2019/141740, WO2019/196904 (for example 3-(3-chlorophenyl)-6-(5-hydroxy-1,3-dimethyl-pyrazole-4-carbonyl)-1,5-dimethyl-quinazoline-2,4-dione and [4-[3-(3-chlorophenyl)-1,5-dimethyl-2,4-dioxo-quinazoline-6-carbonyl]-2,5-dimethyl-pyrazol-3-yl] N,N-diethylcarbamate), WO2019/243358, WO2020/108518 (for example 2-fluoro-N-(5-methyl-1,3,4-oxadiazol-2-yl)-3-[(R)-propylsulfinyl]-4-(trifluoromethyl) benzamide and 2-fluoro-N-(5-methyl-1,3,4-oxadiazol-2-yl)-3-propylsulfinyl-4-(trifluoromethyl) benzamide), WO2020/189576 (for example 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, WO2021/013969, WO2021/094505 and PCT/EP2021/059431.

Table 2 shows the IC50 values for HPPD variants of interest for Mesotrione, Bicyclopyrone, Herbicide C and Herbicide D.

| | | IC50 μM | | | |
|---|---|---|---|---|---|
| SEQUENCE | HPPD | | | Herbicide | Herbicide |
| IDENTITY | Variant | Mesotrione | Bicyclopyrone | C | D |
| SEQ ID NO: | AVESA | 11.52 | 0.38 | 0.25 | 6.73 |
| 122 | V162 | | | | |
| SEQ ID NO: | AVESA | 47.18 | 55.65 | 0.61 | >500 |
| 124 | V180 | | | | |
| SEQ ID NO: | AVESA | 18.56 | — | — | 63.18 |
| 5 | V200 | | | | |
| SEQ ID NO: | AVESA | 24.35 | — | — | 106.40 |
| 17 | V209 | | | | |
| SEQ ID NO: | AVESA | 134.10 | >500 | 0.89 | >500 |
| 8 | V201 | | | | |
| SEQ ID NO: | AVESA | 114.20 | >500 | 1.30 | >500 |
| 20 | V296 | | | | |
| SEQ ID NO: | AVESA | >500 | 39.30 | >50 | 182.50 |
| 14 | V208 | | | | |
| SEQ ID NO: | AVESA | ~50 | 10.21 | >50 | 128.70 |
| 32 | V306 | | | | |
| SEQ ID NO: | AVESA | >500 | — | <0.19 | 262.40 |
| 35 | V307 | | | | |
| SEQ ID NO: | AVESA | 31.50 | <0.39 | ~0.2 | 205.40 |
| 38 | V313 | | | | |
| SEQ ID NO: | AVESA | 346.80 | 101.80 | >50 | 133.60 |
| 44 | V332 | | | | |
| SEQ ID NO: | AVESA | ~50 | 10.23 | >50 | 197.00 |
| 47 | V349 | | | | |
| SEQ ID NO: | AVESA | 136.90 | >500 | 14.29 | >500 |
| 50 | V351 | | | | |
| SEQ ID NO: | AVESA | >500 | 42.10 | 0.28 | 243.40 |
| 53 | V357 | | | | |
| SEQ ID NO: | AVESA | 20.60 | <0.39 | <0.19 | 176.90 |
| 56 | V358 | | | | |
| SEQ ID NO: | APESV | 12.63 | <0.39 | 0.26 | 11.35 |
| 123 | V162 | | | | |
| SEQ ID NO: | APESV | 32.22 | — | — | 85.94 |
| 6 | V200 | | | | |
| SEQ ID NO: | APESV | — | 1.00 | — | — |
| 15 | V208 | | | | |
| SEQ ID NO: | APESV | 44.81 | — | — | 94.98 |
| 18 | V209 | | | | |
| SEQ ID NO: | ALOME | 12.80 | 0.53 | 0.25 | 10.52 |
| 126 | V162 | | | | |
| SEQ ID NO: | ALOME | 31.90 | — | — | 77.81 |
| 7 | V200 | | | | |
| SEQ ID NO: | ALOME | 31.90 | — | — | 96.85 |
| 19 | V209 | | | | |

It is apparent from the IC50 data in Table 2 that in comparison to the Avena control sequence SEQ ID NO: 122, the additional mutations found in one or more variants, e.g. the variants of SEQ ID NO: 50, SEQ ID NO: 8 or SEQ ID NO: 14, show increased tolerance to Mesotrione, Bicyclopyrone, as well as Herbicide D. The equivalent mutations in the Alopecurus and Apera HPPD genes also display a similar increase in IC50 values compared to the control sequences (SEQ ID NO:126 and SEQ ID NO: 123 respectively). It is also seen from Table 2 that the additional mutations found in some of the HPPD variants, e.g. SEQ IDs NO: 20 or SEQ ID NO: 32, can increase the IC50 values to either herbicide C or D or both C and D. An example is SEQ ID NO: 14 which increases the IC50 value for herbicide C by >200 fold or SEQ ID NO: 17 which increases the IC50 value for herbicide D by ˜14 fold or SEQ ID NO: 8 which increases the IC50 value for herbicide D by ˜80 fold.

Using the data in the Table it becomes clear that the different HPPD variants have differing levels of tolerance to each of the herbicides listed; for example SEQ ID NO: 53 has an IC50 fold improvement over SEQ ID NO: 122 of >43× to mesotrione, 110× to Bicyclopyrone, 36× to herbicide D, but no improvement to herbicide C; whereas SEQ ID NO:47 has fold of improvements of 4.3× to Mesotrione, 27 to Bicyclopyrone, >200× to herbicide C and 29× to herbicide D compared to SEQ ID NO: 122.

### Example 2: Construction of Binary Vectors for Transformation of Plants With HPPD Variants

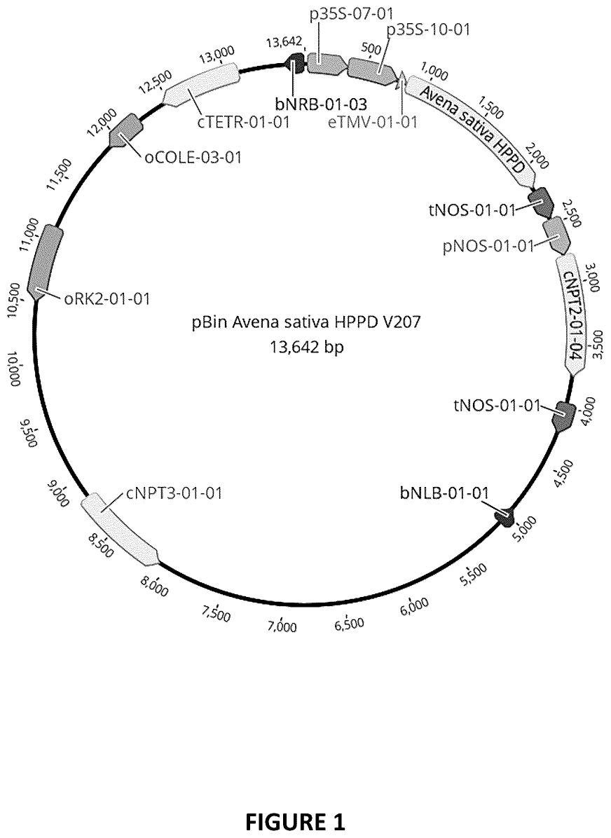

The Binary Vectors described above were constructed using a combination of methods well known to those skilled in the art such as overlap PCR, DNA synthesis, restriction fragment sub-cloning and ligation. Their unique structures are made explicit in FIGS. 1 (vector pBinAvenaSativaHPPDV207), 2 (vector pBinAvenaSativaHPPDV208), and 3 (vector pBinAvenaSativaHPPDV209), and in the sequence listings (SEQ ID NOS: 119-121). Additional information regarding the vectors shown in FIGS. 1-3 are provided below.

The features used in FIG. 1 (vector pBinAvenaSativaHPPDV207) are described as follows: HPPD gene encoding SEQ ID NO: 11—Avena sativa HPPD (Start: 843, End: 2169); Neomycin phosphotransferase—cNPT2-01-04 (Start: 2771 End: 3746); Neomycin phosphotransferase—cNPT3-01-01 (Start: 8048 End: 8839); gene for tetracycline resistance—cTETR-01-01 (Start: 12452 End: 13102); Tobacco Mosaic Virus (TMV) Omega 5′UTR leader sequence—eTMV-01-01 (Start: 773 End: 840); 35S promoter from Cauliflower Mosaic Virus (CaMV)—p35S-07-01 (Start: 21 End: 347); 35S promoter from Cauliflower Mosaic Virus (CaMV)—p35S-10-01 (Start: 348 End: 764); Nos promoter—pNOS-01-01 (Start: 2463 End: 2769); Left border repeat region of T-DNA of Agrobacterium tumefaciens nopaline ti-plasmid—bNLB-01-01 (Start: 4925 End: 5072); Right border repeat region of T-DNA of Agrobacterium tumefaciensnopaline ti-plasmid—bNRB-01-03 (Start: 13476 End: 13637); RK2 origin or replication—oRK2-01-01 (Start: 10496 End: 11113); ColE1 origin of replication—oCOLE-03-01 (Start: 11903 End: 12218); Terminator for Nopaline synthase—tNOS-01-01 (Start: 2196 End: 2450; Also Start: 3965 End: 4219).

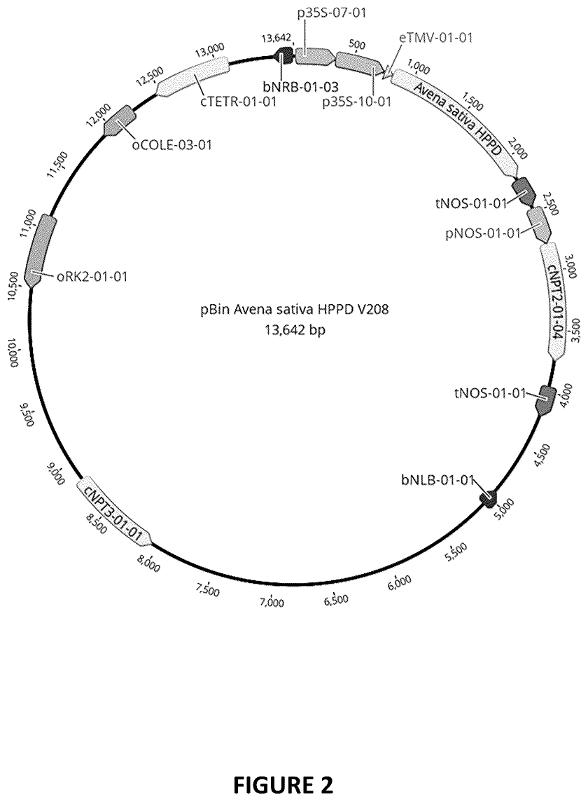

The features used in FIG. 2 (vector pBinAvenaSativaHPPDV208) are described as follows: HPPD gene encoding SEQ ID NO: 14—Avena sativa HPPD (Start: 843, End: 2169); Neomycin phosphotransferase—cNPT2-01-04 (Start: 2771 End: 3746); Neomycin phosphotransferase—cNPT3-01-01 (Start: 8048 End: 8839); gene for tetracycline resistance—cTETR-01-01 (Start: 12452 End: 13102); Tobacco Mosaic Virus (TMV) Omega 5′UTR leader sequence—eTMV-01-01 (Start: 773 End: 840); 35S promoter from Cauliflower Mosaic Virus (CaMV)—p35S-07-01 (Start: 21 End: 347); 35S promoter from Cauliflower Mosaic Virus (CaMV)—p35S-10-01 (Start: 348 End: 764); Nos promoter—pNOS-01-01 (Start: 2463 End: 2769); Left border repeat region of T-DNA of Agrobacterium tumefaciens nopaline ti-plasmid—bNLB-01-01 (Start: 4925 End: 5072); Right border repeat region of T-DNA of Agrobacterium tumefaciens nopaline ti-plasmid—bNRB-01-03 (Start: 13476 End: 13637); RK2 origin or replication—oRK2-01-01 (Start: 10496 End: 11113); ColE1 origin of replication—oCOLE-03-01 (Start: 11903 End: 12218); Terminator for Nopaline synthase—tNOS-01-01 (Start: 2196 End: 2450; Also Start: 3965 End: 4219).

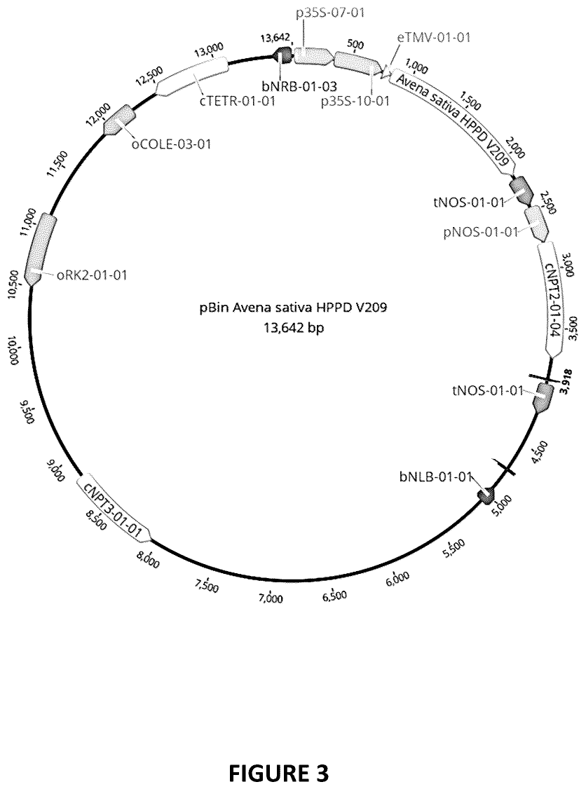

The features used in FIG. 3 (vector pBinAvenaSativaHPPDV209) are described as follows: HPPD gene encoding SEQ ID NO: 17—Avena sativa HPPD (Start: 843, End: 2169); Neomycin phosphotransferase—cNPT2-01-04 (Start: 2771 End: 3746); Neomycin phosphotransferase—cNPT3-01-01 (Start: 8048 End: 8839); gene for tetracycline resistance—cTETR-01-01 (Start: 12452 End: 13102); Tobacco Mosaic Virus (TMV) Omega 5′UTR leader sequence—eTMV-01-01 (Start: 773 End: 840); 35S promoter from Cauliflower Mosaic Virus (CaMV)—p35S-07-01 (Start: 21 End: 347); 35S promoter from Cauliflower Mosaic Virus (CaMV)—p35S-10-01 (Start: 348 End: 764); Nos promoter—pNOS-01-01 (Start: 2463 End: 2769); Left border repeat region of T-DNA of Agrobacterium tumefaciens nopaline ti-plasmid—bNLB-01-01 (Start: 4925 End: 5072); Right border repeat region of T-DNA of Agrobacterium tumefaciens nopaline ti-plasmid—bNRB-01-03 (Start: 13476 End: 13637); RK2 origin or replication—oRK2-01-01 (Start: 10496 End: 11113); ColE1 origin of replication—oCOLE-03-01 (Start: 11903 End: 12218); Terminator for Nopaline synthase—tNOS-01-01 (Start: 2196 End: 2450; Also Start: 3965 End: 4219).

### Example 3: Preparation and Testing of Stable Transgenic Plants Lines Expressing a Heterologous HPPD Enzyme

Avena sativa HPPD or orthologues and variants thereof, for example SEQ IDs 1-63 and 122-127, were expressed in transgenic tobacco. DNA sequences that encode these polypeptides (optimized for tobacco or, optionally, codon optimized according to a target crop such as soybean) were prepared synthetically. Each sequence was designed to include a 5′ fusion with TMV omega 5′ leader sequence such that they are flanked at the 5′ end with XhoI and at the 3′ end with KpnI to facilitate direct cloning into a suitable binary vector for Agrobacterium-based plant transformation.

In one example, the TMV omega 5′ leader and a HPPD encoding gene of interest is excised using XhoI/KpnI and cloned into similarly digested binary vector pBIN 19 (Bevan, Nucl. Acids Res. (1984) behind a double enhanced 35S promoter ahead of a NOS 3′ transcription terminator and then transformed into E. coli DH5 alpha competent cells. DNA recovered from the E. coli is used to transform Agrobacterium tumefaciens LBA4404, and transformed bacteria are selected on media contain rifampicin and kanamycin. Tobacco tissue is subjected to Agrobacterium-mediated transformation using methods well described in the art or as described herein. For example, a master plate of Agrobacterium tumefaciens containing the HPPD expressing the binary vector is used to inoculate 10 ml LB (L broth) containing 100 mg/l Rifampicin plus 50 mg/l Kanamycin using a single bacterial colony. This is incubated overnight at 28° C. shaking at 200 rpm. This entire overnight culture is used to inoculate a 50 ml volume of LB containing the same antibiotics. Again this is cultured overnight at 28° C. shaking at 200 rpm. The Agrobacterium cells are pelleted by centrifuging at 3000 rpm for 15 minutes and then resuspended in MS (Murashige and Skoog) medium containing 30 g/l sucrose, pH 5.9 to an OD (600 nM)=0.6. This suspension is dispensed in 25 ml aliquots into petri dishes.

Clonally micro-propagated tobacco shoot cultures are used to excise young (not yet fully expanded) leaves. The mid rib and outer leaf margins are removed and discarded, and the remaining lamina cut into 1 cm squares. These are transferred to the Agrobacterium suspension for 20 minutes. Explants are then removed, dabbed on sterile filter paper to remove excess suspension, then transferred onto solid NBM medium (MS medium containing 30 g/l sucrose, 1 mg/l BAP (benzylaminopurine) and 0.1 mg/l NAA (napthalene acetic acid) at pH 5.9 and solidified with 8 g/l Plantagar), with the abaxial surface of each explant in contact with the medium. Approximately 7 explants are transferred per plate, which are then sealed and maintained in a lit incubator at 25° C. for a 16 hour photoperiod for 3 days.

Explants are then transferred onto NBM medium containing 100 mg/l Kanamycin plus antibiotics to prevent further growth of Agrobacterium (200 mg/l timentin with 250 mg/l carbenicillin). Further subculture onto this same medium was then performed every 2 weeks.

As shoots start to regenerate from the callusing leaf explants, these are removed to Shoot elongation medium (MS medium, 30 g/l sucrose, 8 g/l Plantagar, 100 mg/l Kanamycin, 200 mg/l timentin, 250 mg/l carbenicillin, pH 5.9). Stable transgenic plants readily root within 2 weeks. To provide multiple plants per event to ultimately allow more than one herbicide test per transgenic plant, all rooting shoots are micropropagated to generate 3 or more rooted clones.

Putative transgenic plants that are rooting and showing vigorous shoot growth on the medium incorporating Kanamycin are analysed by PCR using primers that amplified a 500bp fragment within the HPPD transgene. Evaluation of this same primer set on untransformed tobacco showed conclusively that these primers would not amplify sequences from the native tobacco HPPD gene.

Transformed shoots are divided into 2 or 3 clones and regenerated from kanamycin resistant callus. Shoots are rooted on MS agar containing kanamycin. Surviving rooted explants are re-rooted to provide approximately 50 kanamycin resistant events represented by about 3 clonal plantlets from each event.

Once rooted, plantlets are transferred from agar and potted into 50% peat, 50% John Innes Soil No. 3 or, for example, MetroMix® 380 soil (Sun Gro Horticulture, Bellevue, WA) with slow-release fertilizer in 3 inch round or 4 inch square pots and left regularly watered to establish for 8-12d in the glass house. Glass house conditions are about 24-27° C. day; 18-21° C. night and approximately a 14h (or longer in UK summer) photoperiod. Humidity is adjusted to ˜65% and light levels used are up to 2000 umol/m² at bench level. Once new tissue emerged and plants reach the 2-4 leaf stage, some of the clones from each event are sprayed with an HPPD inhibitor of interest selected from the herbicides listed in reference to Example 1. For example, plants are sprayed with rates of from 150 -800 g/ha of mesotrione. For example Callisto® is mixed in water with 0.2-0.25% X-77 surfactant and sprayed from a boom on a suitable track sprayer moving at 2 mph in a DeVries spray chamber (Hollandale, MN) with the nozzle about 2 inches from the plant tops. Spray volume is suitably 25 gallons per acre or, for example, 200 l/ha at a rate of 150g of mesotrione/ha.

Plants are assessed for damage and scored at, for example, 7 and 14 days after treatment (DAT). About 20-30 TO events are produced for a number of HPPD variant genes. The results were obtained (average resistance level, number of plants exhibiting less than 10% damage at a given rate, etc.) for each HPPD variant and, accordingly, each set is scored for resistance and compared with the results obtained with expression of the corresponding native HPPD (SEQ ID NOs: 1-2) as well as the corresponding base HPPD variants SEQ IDs 122-125 (used as control or reference). It may be found for example that expression of some HPPDs results in plants that exhibit substantial resistance to HPPD inhibitors and furthermore the herbicide resistance is significantly (1.4-2×) enhanced over that conferred by like expression of the control or reference sequences SEQ ID NOs: 122-125.

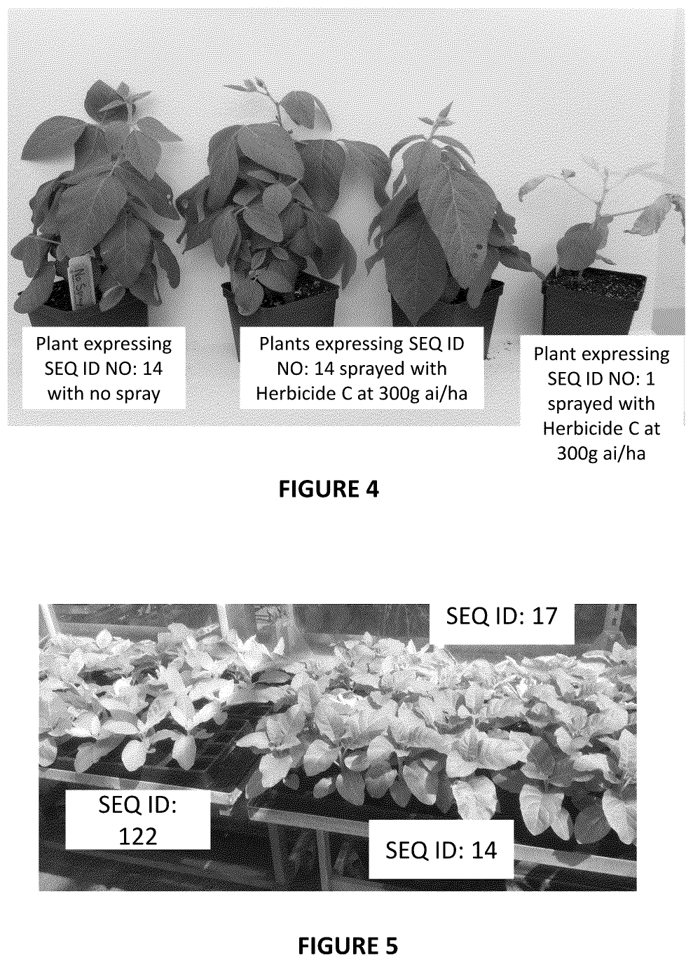

FIG. 4 shows a comparison between transgenic plants expressing multiple copies of SEQ ID NO: 14 sprayed with Herbicide C at 300 ai/ha relative to a no-spray control. In addition, the transgenic plants are compared to a transgenic plant expressing the wild-type HPPD gene (SEQ ID NO: 1) from which the HPPD variant of SEQ ID NO: 14 is derived. Almost no chlorosis was observed in some of the multi-copy events transformed with the HPPD variant SEQ ID NO: 14. In addition, there was significantly reduced stunting compared to the plants expressing the wild-type HPPD.

Plants of events showing the least damage are grown to flowering, then bagged and allowed to self. The seed from selected events are collected and sown again in pots, and tested again for herbicide resistance in a spray test for resistance to HPPD herbicide (for example mesotrione). Single copy events amongst the Ti plant lines are identified by their 3:1 segregation ratio (with respect to kanamycin and/or herbicide) and by quantitative RT-PCR. Seed from the thus selected Ti tobacco (var. Samsun) lines are sown in 3 inch diameter pots containing 50% peat and 50% John Innes Soil No. 3. After growth to the 3 leaf stage, plants are sprayed as described above in order to test for herbicide tolerance relative to like-treated non-transgenic tobacco plants, and also in comparison with like-treated Ti plants expressing the base HPPD SEQ IDs 122-125. HPPD expression levels were monitored by Western analysis.

TABLE 3.1

| SEQ ID 122 | | SEQ ID 14 | |
|---|---|---|---|
| Event ID | Herbicide C 100 g/ha | Event ID | Herbicide C 100 g/ha |
| 3526 | 95 | 2095 | 0 |
| 3527 | 90 | 2096 | 5 |
| 3530 | 90 | 2098 | 20 |
| 3531 | 90 | 2099 | 80 |
| 3532 | 90 | 2102 | 0 |
| 3533 | 90 | 2103 | 85 |
| 3535 | 90 | 2114 | 85 |
| 3536 | 70 | 2115 | 70 |
| 3537 | 95 | 2116 | 15 |
| 3538 | 96 | 2118 | 65 |
| 3541 | 95 | 2122 | 85 |
| 3552 | 95 | 2123 | 30 |
| 3554 | 95 | 2126 | 20 |
| 3557 | 95 | 2128 | 25 |
| 3561 | 90 | 2130 | 30 |
| 3563 | 90 | 2132 | 70 |
| 3565 | 90 | 2133 | 20 |
| 3572 | 95 | 2134 | 85 |
| 3576 | 95 | 2136 | 20 |
| 3591 | 96 | 2138 | 55 |
| | | 2143 | 40 |
| | | 2144 | 85 |
| | | 2145 | 0 |
| | | 2152 | 70 |
| | | 2156 | 10 |
| | | 2160 | 10 |
| | | 2166 | 15 |
| | | 2167 | 85 |
| | | 2168 | 80 |
| | | 2169 | 35 |

Table 3.1 shows the herbicidal damage scoring on transgenic tobacco plants 7 days after treatment. A score of 100=complete death and 0=no damage observed.

Tobacco plants expressing either SEQ ID NO: 122 or SEQ ID NO: 14 were sprayed with herbicide C at a rate of equivalent to 100g/ha. It is clear from the results that many lines expressing SEQ ID NO: 14 show significantly improved tolerance to the herbicide in comparison to SEQ ID NO: 122 with several showing less than 10% damage.

FIG. 5 shows an example comparison between tobacco plants expressing either SEQ ID NO: 122 or SEQ ID NO: 14 or SEQ ID NO: 17 that were sprayed with herbicide C at a rate of equivalent to 50 g/ha. Transgenic plants expressing SEQ ID NO: 14 displayed significantly reduced chlorosis (yellowing of leaf tissue) as compared to the reference plant expressing SEQ ID NO: 122 as well as transgenic plants expressing an alternate HPPD mutant SEQ ID NO: 17.

In a further spray test the best events from each population were then treated with either 800 g/ha Mesotrione, 400 g/ha Tembotrione or 400 g/ha Isoxaflutole. The herbicide damage scores are shown in Table 3.2.

TABLE 3.2

| SEQ ID 122 | | | | SEQ ID 14 | | | |
|---|---|---|---|---|---|---|---|
| Event | Mesotrione | Tembotrione | Isoxaflutole | Event | Mesotrione | Tembotrione | Isoxaflutole |
| ID | (800 gai/ha) | (400 gai/ha) | (400 gai/ha) | ID | (800 gai/ha) | (400 gai/ha) | (400 gai/ha) |
| 3526 | 0 | 55 | 25 | 2095 | 0 | 25 | 5 |
| 3532 | 5 | 65 | 50 | 2102 | 0 | 30 | 0 |
| 3535 | 0 | 40 | 5 | 2133 | 0 | 25 | 5 |
| 3536 | 0 | 50 | 5 | 2145 | 0 | 25 | 5 |
| 3537 | 0 | 45 | 10 | 2156 | 0 | 25 | 0 |
| 3538 | 10 | 50 | 5 | | | | |
| 3541 | 0 | 55 | 15 | | | | |
| 3554 | 1 | 55 | 10 | | | | |
| 3561 | 0 | 45 | 10 | | | | |
| 3576 | 0 | 45 | 15 | | | | |

Table 3.2 shows the herbicidal damage scoring on transgenic tobacco plants 14 days after treatment. A score of 100=complete death and 0=no damage observed.

The results show that the plants expressing SEQ ID 14 are equally tolerant to Mesotrione and Isoxaflutole as those expressing SEQ ID 122. The results from the Tembotrione spray show that the SEQ ID 14 expressing plants had increased tolerance compared to SEQ ID 122 expressing plants.

The tobacco lines expressing SEQ ID 122 were now compared to a population of tobacco which express SEQ ID 20. The plants were sprayed with 800g/ha Bicyclopyrone and scored for damage. Table 3.3 shows the damage scorings.

TABLE 3.3

| SEQ ID 122 | | SEQ ID 20 | |
|---|---|---|---|
| Event ID | Bicyclopyrone 800 g/ha | Event ID | Bicyclopyrone 800 g/ha |
| 3526 | 60 | 3267 | 0 |
| 3527 | 55 | 3268 | 85 |
| 3530 | 55 | 3269 | 75 |
| 3531 | 55 | 3276 | 65 |
| 3532 | 70 | 3281 | 10 |
| 3533 | 60 | 3283 | 55 |
| 3535 | 55 | 3284 | 60 |
| 3536 | 40 | 3292 | 50 |
| 3537 | 60 | 3295 | 40 |
| 3538 | 50 | 3298 | 65 |
| 3541 | 55 | 3303 | 10 |
| 3552 | 55 | 3315 | 65 |
| 3554 | 65 | 3317 | 75 |
| 3557 | 60 | 3319 | 40 |
| 3561 | 35 | 3322 | 65 |
| 3563 | 65 | 3326 | 30 |
| 3565 | 45 | 3327 | 80 |
| 3572 | 55 | 3333 | 70 |
| 3576 | 55 | 3338 | 25 |
| 3591 | 75 | 3343 | 50 |

Table 3.3 shows the herbicidal damage scoring on transgenic tobacco plants 14 days after treatment. A score of 100=complete death and 0=no damage observed.

The results demonstrated that, in comparison to events expressing SEQ ID 122, several events that express SEQ ID 20 have increased tolerance to Bicyclopyrone e.g. events # 3267 and 3281.

In a further experiment, transgenic tobacco events were created which express sequence SEQ ID 125 or SEQ ID 9. These plants were then sprayed with either 100 g/ha or 200 g/ha of herbicide C.

TABLE 3.4

| SEQ ID 125 | | | SEQ ID 9 | | |
|---|---|---|---|---|---|
| Event | Herbicide C | Herbicide C | Event | Herbicide C | Herbicide C |
| ID | 100 g/ha | 200 g/ha | ID | 100 g/ha | 200 g/ha |
| 3444 | 65 | 85 | 2864 | 75 | 90 |
| 3458 | 70 | 85 | 2868 | 10 | 45 |
| 3462 | 10 | 80 | 2874 | 10 | 40 |
| 3468 | 10 | 85 | 2889 | 70 | 70 |
| 3473 | 10 | 60 | 2898 | 25 | 45 |
| 3478 | 15 | 80 | 2904 | 10 | 35 |
| 3482 | 5 | 75 | 2913 | 5 | 15 |
| 3484 | 20 | 80 | 2923 | 75 | 85 |
| 3493 | 50 | 85 | 2926 | 80 | 99 |
| 3503 | 70 | 85 | 2928 | 10 | 45 |

Table 3.4 shows the herbicidal damage scoring on transgenic tobacco plants 14 days after treatment. A score of 100=complete death and 0=no damage observed.

The results of the spray test demonstrate that plants expressing either SEQ ID 125 or SEQ ID 9 display tolerance to 100 g/ha herbicide C. However those plants expressing SEQ ID 9 demonstrate higher tolerance to 200 g/ha herbicide C compared to those expressing SEQ ID 125 (e.g. event #3482).

### Example 4: Preparation and Testing of Stable Transgenic Plants Lines Expressing a Heterologous HPPD Enzyme

Transgenic plants expressing wild type Avena sativa HPPD or orthologues and variants thereof, were generated using the methods described previously in Example 3. Tables 4.1-4.9 show the herbicidal damage scoring on transgenic tobacco plants 7 days after treatment. A score of 100=complete death and 0=no damage observed.

TABLE 4.1
Spray test data for wild-type tobacco plants

| | Mesotrione | Tembotrione | Isoxaflutole | Herbicide C | Herbicide C | Bicyclopyrone | Topramezone | Herbicide D |
|---|---|---|---|---|---|---|---|---|
| Event | (800 gai/ha) | (400 gai/ha) | (400 gai/ha) | (100 g/ha) | (200 g/ha) | (800 g/ha) | (100 g/ha) | (2000 g/ha) |
| 1 | 95 | 95 | 95 | 95 | 95 | 95 | 99 | 96 |
| 2 | 95 | 97 | 95 | 95 | 95 | 95 | 97 | 95 |
| 3 | 95 | 98 | 95 | 95 | 96 | 95 | 97 | 95 |
| 4 | 95 | 97 | 95 | 95 | 95 | 95 | 95 | 95 |
| 5 | 95 | 97 | 95 | 95 | 95 | 95 | 98 | 99 |
| 6 | 95 | 97 | 97 | 99 | 95 | 95 | 98 | 96 |
| 7 | 95 | 97 | 95 | 96 | 95 | 95 | 97 | 97 |
| 8 | 95 | 97 | 95 | 95 | 97 | 95 | 97 | 97 |
| 9 | 95 | 97 | 95 | 95 | 98 | 95 | nt | 96 |
| 10 | 97 | 97 | 96 | 95 | 97 | 95 | nt | nt |

TABLE 4.2
Spray test data for plants expressing mutant HPPD (SEQ ID NO: 124)

| | Mesotrione | Isoxaflutole | Herbicide C | Herbicide C | Bicyclopyrone | Topramezone | Herbicide D |
|---|---|---|---|---|---|---|---|
| Event | (800 gai/ha) | (400 gai/ha) | (100 g/ha) | (200 g/ha) | (800 g/ha) | (100 g/ha) | (2000 g/ha) |
| 3619 | 5 | 5 | 60 | 65 | 5 | 0 | 20 |
| 3625 | 0 | 5 | 60 | 65 | 0 | 0 | 70 |
| 3626 | 0 | 5 | 35 | 50 | 0 | 0 | 65 |
| 3637 | 0 | 0 | 65 | 60 | 0 | 0 | 70 |
| 3665 | 0 | 5 | 60 | 60 | 0 | 0 | 65 |
| 3676 | 5 | 5 | 60 | 65 | 10 | 0 | 60 |
| 3681 | 0 | 5 | 65 | 65 | 0 | 0 | 25 |
| 3682 | 0 | 5 | 60 | 65 | 5 | 0 | 55 |
| 3685 | 0 | 5 | 55 | 65 | 0 | 0 | 30 |
| 3686 | 0 | 5 | 25 | 65 | 0 | 0 | 50 |

TABLE 4.3
Spray test data for plants expressing mutant HPPD (SEQ ID NO: 125)

| | Mesotrione | Isoxaflutole | Herbicide C | Herbicide C | Bicyclopyrone | Topramezone | Herbicide D |
|---|---|---|---|---|---|---|---|
| Event | (800 gai/ha) | (400 gai/ha) | (100 g/ha) | (200 g/ha) | (800 g/ha) | (100 g/ha) | (2000 g/ha) |
| 3444 | 0 | 5 | 65 | 85 | 5 | 0 | 45 |
| 3458 | 0 | 0 | 70 | 85 | 5 | 0 | 70 |
| 3462 | 5 | 0 | 10 | 80 | 0 | 0 | 20 |
| 3468 | 0 | 0 | 10 | 85 | 0 | 0 | 60 |
| 3473 | 0 | 0 | 10 | 60 | 5 | 0 | 60 |
| 3478 | 0 | 0 | 15 | 80 | 10 | 0 | 25 |
| 3482 | 0 | 0 | 5 | 75 | 5 | 0 | 75 |
| 3484 | 0 | 0 | 20 | 80 | 0 | 0 | 40 |
| 3493 | 0 | 5 | 50 | 85 | 5 | 0 | 25 |
| 3503 | 0 | 0 | 70 | 85 | 0 | 0 | 25 |

TABLE 4.4
Spray test data for plants expressing mutant HPPD (SEQ ID NO: 122)

| | Mesotrione | Isoxaflutole | Herbicide C | Herbicide C | Bicyclopyrone | Topramezone | Herbicide D |
|---|---|---|---|---|---|---|---|
| Event | (800 gai/ha) | (400 gai/ha) | (100 g/ha) | (200 g/ha) | (800 g/ha) | (100 g/ha) | (2000 g/ha) |
| 3526 | 0 | 25 | 95 | 95 | 60 | 0 | 85 |
| 3532 | 5 | 50 | 95 | 95 | 70 | 0 | 70 |
| 3535 | 0 | 5 | 35 | 95 | 55 | 0 | 60 |
| 3536 | 0 | 5 | 45 | 95 | 40 | 0 | 65 |
| 3537 | 0 | 10 | 95 | 99 | 60 | 0 | 80 |
| 3538 | 10 | 5 | 80 | 95 | 50 | 0 | 70 |
| 3541 | 0 | 15 | 95 | 97 | 55 | 0 | 65 |
| 3554 | 1 | 10 | 96 | 95 | 65 | 0 | 75 |
| 3561 | 0 | 10 | 95 | 98 | 35 | 0 | 75 |
| 3576 | 0 | 15 | 95 | 95 | 55 | 0 | 80 |

TABLE 4.5
Spray test data for plants expressing mutant HPPD (SEQ ID NO: 8)

| | Mesotrione | Isoxaflutole | Herbicide C | Herbicide C | Bicyclopyrone | Topramezone | Herbicide D |
|---|---|---|---|---|---|---|---|
| Event | (800 gai/ha) | (400 gai/ha) | (100 g/ha) | (200 g/ha) | (800 g/ha) | (100 g/ha) | (2000 g/ha) |
| 3191 | 0 | 10 | 10 | 85 | 65 | 0 | 5 |
| 3200 | 60 | 30 | 70 | 85 | 60 | 10 | 5 |
| 3202 | 5 | 10 | 5 | 80 | 10 | 0 | 5 |
| 3219 | 30 | 5 | 10 | 40 | 10 | 0 | 10 |
| 3224 | 0 | 5 | 35 | 75 | 30 | 0 | 5 |
| 3225 | 0 | 30 | 70 | 85 | 20 | 0 | 5 |
| 3227 | 10 | 25 | 70 | 80 | 50 | 0 | 5 |
| 3236 | 0 | 20 | 65 | 80 | 25 | 0 | 10 |
| 3260 | 5 | 20 | 80 | 95 | 30 | 5 | 5 |
| 3266 | 30 | 20 | 85 | 85 | 50 | 0 | 5 |

TABLE 4.6
Spray test data for plants expressing mutant HPPD (SEQ ID NO: 9)

| | Mesotrione | Isoxaflutole | Herbicide C | Herbicide C | Bicyclopyrone | Topramezone | Herbicide D |
|---|---|---|---|---|---|---|---|
| Event | (800 gai/ha) | (400 gai/ha) | (100 g/ha) | (200 g/ha) | (800 g/ha) | (100 g/ha) | (2000 g/ha) |
| 2864 | 5 | 20 | 75 | 90 | 5 | 0 | 5 |
| 2868 | 5 | 0 | 10 | 45 | 5 | 0 | 0 |
| 2874 | 0 | 5 | 10 | 40 | 0 | 0 | 0 |
| 2889 | 65 | 75 | 70 | 70 | 10 | 35 | 40 |
| 2898 | 10 | 10 | 25 | 45 | 25 | 80 | 5 |
| 2904 | 5 | 5 | 10 | 35 | 5 | 0 | 0 |
| 2913 | 0 | 0 | 5 | 15 | 5 | 5 | 0 |
| 2923 | 65 | 65 | 75 | 85 | 50 | nt | 20 |
| 2926 | 70 | 65 | 80 | 99 | 70 | 90 | 85 |
| 2928 | 40 | 10 | 10 | 45 | 0 | 10 | 5 |

TABLE 4.7
Spray test data for plants expressing mutant HPPD (SEQ ID NO: 21)

| | Mesotrione | Isoxaflutole | Herbicide C | Herbicide C | Bicyclopyrone | Topramezone | Herbicide D |
|---|---|---|---|---|---|---|---|
| Event | (800 gai/ha) | (400 gai/ha) | (100 g/ha) | (200 g/ha) | (800 g/ha) | (100 g/ha) | (2000 g/ha) |
| 2933 | 0 | 5 | 5 | 40 | 5 | 65 | 85 |
| 2936 | 0 | 0 | 10 | 75 | 0 | 0 | 0 |
| 2938 | 0 | 5 | 10 | 99 | 5 | 0 | 0 |
| 2962 | 0 | 5 | 75 | 90 | 5 | 0 | 0 |
| 2969 | 0 | 5 | 25 | 45 | 5 | 0 | 10 |
| 2975 | 10 | 25 | 90 | 90 | 0 | 0 | 0 |
| 2983 | 5 | 0 | 30 | 85 | 5 | 0 | 0 |
| 2992 | 10 | 5 | 45 | 80 | 0 | 0 | 0 |
| 3004 | 5 | 5 | 25 | 85 | 0 | 0 | 0 |
| 3013 | 0 | 5 | 25 | 85 | 0 | 0 | 0 |

TABLE 4.8
Spray test data for plants expressing mutant HPPD (SEQ ID NO: 14)

| | | | Herbicide | Bicyclo- | |
|---|---|---|---|---|---|
| | Mesotrione | Isoxaflutole | C | pyrone | Topramezone |
| Event | (800 gai/ha) | (400 gai/ha) | (200 g/ha) | (800 g/ha) | (100 g/ha) |
| 2095 | 0 | 5 | 10 | 0 | nt |
| 2102 | 0 | 0 | 20 | 0 | nt |
| 2133 | 0 | 5 | 35 | 0 | nt |
| 2145 | 0 | 5 | 20 | 0 | nt |
| 2156 | 0 | 0 | 35 | 0 | nt |

TABLE 4.9
Spray test data for plants expressing mutant HPPD (SEQ ID NO: 15)

| | Mesotrione | Tembotrione | Isoxaflutole | | Mesotrione | Tembotrione | Isoxaflutole |
|---|---|---|---|---|---|---|---|
| Event | (800 gai/ha) | (400 gai/ha) | (400 gai/ha) | Event | (800 gai/ha) | (400 gai/ha) | (400 gai/ha) |
| 4528 | 0 | 0 | 0 | 4556 | 0 | 0 | 5 |
| 4529 | 0 | 0 | 0 | 4557 | 10 | 0 | 0 |
| 4530 | 0 | 0 | 0 | 4562 | 0 | 0 | 5 |
| 4531 | 0 | 0 | 0 | 4563 | 0 | 0 | 0 |
| 4532 | 0 | 0 | 0 | 4564 | 0 | 0 | 0 |
| 4534 | 0 | 0 | 0 | 4565 | 0 | 0 | 0 |
| 4536 | 35 | 0 | 0 | 4567 | 20 | 0 | 1 |
| 4537 | 0 | 0 | 5 | 4575 | 0 | 0 | 0 |
| 4538 | 1 | 0 | 0 | 4576 | 0 | 0 | 5 |
| 4540 | 0 | 0 | 5 | 4579 | 0 | 0 | 5 |
| 4541 | 5 | 0 | 0 | 4585 | 5 | 0 | 0 |
| 4542 | 0 | 0 | 5 | 4601 | 5 | 0 | 0 |
| 4544 | 0 | 0 | 0 | 4603 | 0 | 0 | 0 |
| 4547 | 0 | 0 | 0 | 4607 | 85 | 65 | 80 |
| 4552 | 0 | 0 | 0 | 4609 | 0 | 0 | 0 |

A number of conclusions were derived from the data in Tables 4.1-4.9. The properties of the mutant HPPDs of SEQ ID NOs: 4-63 and 122-127 indicated that certain substitutions at certain positions provided significant improvements in herbicide tolerance relative to the corresponding control or reference HPPD sequence from which the mutant was derived (e.g., SEQ ID NOs: 122-125). As shown by the lower damage rating, the transgenic plants expressing mutant HPPDs comprising one or more of the mutations at the positions disclosed at Table 1 had significantly improved herbicide tolerance to one or more of known HPPD herbicides such as mesotrione, tembotrione, isoxaflutole, and bicyclopyrone.

Tobacco plants expressing the mutant HPPD SEQ ID 14 displayed reduced damage when sprayed with 200 g/ha of Herbicide C compared to tobacco plants expressing HPPD SEQ IDs 122, 124 or 125. Tobacco plants expressing the mutant HPPDs SEQ ID 8, 9 or 21 displayed significantly reduced damage when sprayed with 2000 g/ha of Herbicide D compared to tobacco plants expressing HPPD SEQ IDs 122, 124 or 125. Many of the transgenic events expressing the mutant HPPDs SEQ ID 8, 9 or 21 exhibit no damage at all following spray with 200 0g/ha of Herbicide D. This demonstrates the enhanced tolerance of these events to herbicides C and D compared to the corresponding controls.

Thus, a number of the new HPPD sequences described herein offer significant improvements over the prior art in respect of providing better options for providing tolerance to HPPD herbicides and especially in respect of the chemical classes exemplified.

All patents, patent applications and publications mentioned in the specification are indicative of the level of those skilled in the art to which this invention pertains. All patents, patent applications and publications are herein incorporated by reference to the same extent as if each individual publication or patent application was specifically and individually indicated to be incorporated by reference.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

TABLE 5
Summary of sequences in sequence listing

| SEQ ID NO: | DNA/PRT | Description |
|---|---|---|
| SEQ ID NO: 1 | PRT | Native HPPD |
| SEQ ID NO: 2 | PRT | Native HPPD |
| SEQ ID NO: 3 | PRT | Native HPPD |
| SEQ ID NO: 4 | PRT | V0, Avena sativa A111 deletion |
| SEQ ID NO: 5 | PRT | V200, Avena sativa, V218I + V260A + A327R + 1340E + L359M |
| SEQ ID NO: 6 | PRT | V200, Apera spica-venti, V218I + V260A + A327R + 1340E + L368M |
| SEQ ID NO: 7 | PRT | V200, Alopecurus myosuroides, V218I + V260A + A327R + 1340E + L359M |
| SEQ ID NO: 8 | PRT | V201, Avena sativa, V218I + V260A + A327R + 1340E + L359M + G411A |
| SEQ ID NO: 9 | PRT | V201, Apera spica-venti, V218I + V260A + A327R + 1340E + L359M + G411A |
| SEQ ID NO: 10 | PRT | V201, Alopecurus myosuroides, V218I + V260A + A327R + 1340E + L359M + G411A |
| SEQ ID NO: 11 | PRT | V207, Avena sativa, V218I + V260T + A327R + 1340E + L359M |
| SEQ ID NO: 12 | PRT | V207, Apera spica-venti, V218I + V260T + A327R + 1340E + L359M |
| SEQ ID NO: 13 | PRT | V207, Alopecurus myosuroides, V2171 + V260T + A326R + 1339E + L358M |
| SEQ ID NO: 14 | PRT | V208, Avena sativa, V218I + P271N + A327R + 1340E + L359M |
| SEQ ID NO: 15 | PRT | V208, Apera spica-venti, V218I + P271N + A327R + 1340E + L359M |
| SEQ ID NO: 16 | PRT | V208, Alopecurus myosuroides, V218I + P271N + A327R + 1340E + L359M |
| SEQ ID NO: 17 | PRT | V209, Avena sativa, R214G + V218I + V260A + A327R + 1340E + L359M |
| SEQ ID NO: 18 | PRT | V209, Apera spica-venti, R214G + V218I + V260A + A327R + 1340E + L359M |
| SEQ ID NO: 19 | PRT | V209, Alopecurus myosuroides, R214G + V218I + V260A + A327R + 1340E + L359M |
| SEQ ID NO: 20 | PRT | V296, Avena sativa, R214G + V218I + A327R + 1340E + L359Y + G411A |
| SEQ ID NO: 21 | PRT | V296, Apera spica-venti, R214G + V218I + A327R + 1340E + L359Y + G411A |
| SEQ ID NO: 22 | PRT | V296, Alopecurus myosuroides, R214G + V218I + A327R + 1340E + L359Y + G411A |
| SEQ ID NO: 23 | PRT | V300, Avena sativa, R214G + V218I + V260A + A327R + 1340E + L359Y + G411A |
| SEQ ID NO: 24 | PRT | V300, Apera spica-venti, R214G + V218I + V260A + A327R + 1340E + L359Y + G411A |
| SEQ ID NO: 25 | PRT | V300, Alopecurus myosuroides, R214G + V218I + V260A + A327R + 1340E + L359Y + G411A |
| SEQ ID NO: 26 | PRT | V301, Avena sativa, R214G + V218I + V260A + A327R + 1340E + L359M + G411A |
| SEQ ID NO: 27 | PRT | V301, Apera spica-venti, R214G + V218I + V260A + A327R + 1340E + L359M + G411A |
| SEQ ID NO: 28 | PRT | V301, Alopecurus myosuroides, R214G + V218I + V260A + A327R + 1340E + L359M + G411A |
| SEQ ID NO: 29 | PRT | V304, Avena sativa, R214G + V218I + V260A + P271N + A327R + 1340E + L359M + G411A |
| SEQ ID NO: 30 | PRT | V304, Apera spica-venti, R214G + V218I + V260A + P271N + A327R + 1340E + L359M + G411A |
| SEQ ID NO: 31 | PRT | V304, Alopecurus myosuroides, R214G + V218I + V260A + P271N + A327R + 1340E + L359M + G411A |
| SEQ ID NO: 32 | PRT | V306, Avena sativa, R214G + V218I + P271N + A327R + 1340E + L359M |
| SEQ ID NO: 33 | PRT | V306, Apera spica-venti, R214G + V218I + P271N + A327R + 1340E + L359M |
| SEQ ID NO: 34 | PRT | V306, Alopecurus myosuroides, R214G + V218I + P271N + A327R + 1340E + L359M |
| SEQ ID NO: 35 | PRT | V307, Avena sativa, R214G + V218I + V260A + P271N + A327R + 1340E + L359M |
| SEQ ID NO: 36 | PRT | V307, Apera spica-venti, R214G + V218I + V260A + P271N + A327R + 1340E + L359M |
| SEQ ID NO: 37 | PRT | V307, Alopecurus myosuroides, R214G + V218I + V260A + P271N + A327R + 1340E + L359M |
| SEQ ID NO: 38 | PRT | V313, Avena sativa, R214G + V218I + V260A + P271N + A327R + 1340E + L359Y |
| SEQ ID NO: 39 | PRT | V313, Apera spica-venti, R214G + V218I + V260A + P271N + A327R + 1340E + L359Y |
| SEQ ID NO: 40 | PRT | V313, Alopecurus myosuroides, R214G + V218I + V260A + P271N + A327R + 1340E + L359Y |
| SEQ ID NO: 41 | PRT | V316, Avena sativa, R214G + V218I + V260T + A327R + 1340E + L359M + G411A |
| SEQ ID NO: 42 | PRT | V316, Apera spica-venti, R214G + V218I + V260T + A327R + 1340E + L359M + G411A |
| SEQ ID NO: 43 | PR | V316, Alopecurus myosuroides, R214G + V218I + V260T + A327R + 1340E + L359M + G411A |
| SEQ ID NO: 44 | PRT | V332, Avena sativa, R214G + V218I + V260A + P271N + A327R + 1340E + L359M + K404N |
| SEQ ID NO: 45 | PRT | V332, Apera spica-venti, R214G + V218I + V260A + P271N + A327R + 1340E + L359M + K404N |
| SEQ ID NO: 46 | PRT | V332, Alopecurus myosuroides, R214G + V218I + V260A + P271N + A327R + 1340E + L359M + K404N |
| SEQ ID NO: 47 | PRT | V349, Avena sativa, V218I + P271N + S304T + A327R + 1340E + L359M |
| SEQ ID NO: 48 | PRT | V349, Apera spica-venti, V218I + P271N + S304T + A327R + 1340E + L359M |
| SEQ ID NO: 49 | PRT | V349, Alopecurus myosuroides, V218I + P271N + S304T + A327R + 1340E + L359M |
| SEQ ID NO: 50 | PRT | V351, Avena sativa, R214G + V218I + S304T + A327R + 1340E + L359Y + G411A |
| SEQ ID NO: 51 | PRT | V351, Apera spica-venti, R214G + V218I + S304T + A327R + 1340E + L359Y + G411A |
| SEQ ID NO: 52 | PRT | V351, Alopecurus myosuroides, R214G + V218I + S304T + A327R + 1340E + L359Y + G411A |
| SEQ ID NO: 53 | PRT | V357, Avena sativa, R214G + V218I + P271N + S304T + A327R + 1340E + L359M |
| SEQ ID NO: 54 | PRT | V357, Apera spica-venti, R214G + V218I + P271N + S304T + A327R + 1340E + L359M |
| SEQ ID NO: 55 | PRT | V357, Alopecurus myosuroides, R214G + V218I + P271N + S304T + A327R + 1340E + L359M |
| SEQ ID NO: 56 | PRT | V358, Avena sativa, R214G + V218I + V260A + P271N + S304T + A327R + 1340E + L359M |
| SEQ ID NO: 57 | PRT | V358, Apera spica-venti, R214G + V218I + V260A + P271N + S304T + A327R + 1340E + L359M |
| SEQ ID NO: 58 | PRT | V358, Alopecurus myosuroides, R214G + V218I + V260A + P271N + S304T + A327R + 1340E + L359M |
| SEQ ID NO: 59 | PRT | R214; X1, X2, X3, X4, X5, X6, X7, X8, X9 |
| SEQ ID NO: 60 | PRT | V260; X1, X2, X3, X4, X5, X6, X7, X8, X9 |
| SEQ ID NO: 61 | PRT | P271; X1, X2, X3, X4, X5, X6, X7, X8, X9 |
| SEQ ID NO: 62 | PRT | S304; X1, X2, X3, X4, X5, X6, X7, X8, X9 |
| SEQ ID NO: 63 | PRT | K404; X1, X2, X3, X4, X5, X6, X7, X8, X9 |
| SEQ ID NO: 64 | DNA | V0, Avena sativa A111 deletion |
| SEQ ID NO: 65 | DNA | V200, Avena sativa, V218I + V260A + A327R + 1340E + L359M |
| SEQ ID NO: 66 | DNA | V200, Apera spica-venti, V218I + V260A + A327R + 1340E + L368M |
| SEQ ID NO: 67 | DNA | V200, Alopecurus myosuroides, V218I + V260A + A327R + 1340E + L359M |
| SEQ ID NO: 68 | DNA | V201, Avena sativa, V218I + V260A + A327R + 1340E + L359M + G411A |
| SEQ ID NO: 69 | DNA | V201, Apera spica-venti, V218I + V260A + A327R + 1340E + L359M + G411A |
| SEQ ID NO: 70 | DNA | V201, Alopecurus myosuroides, V218I + V260A + A327R + 1340E + L359M + G411A |
| SEQ ID NO: 71 | DNA | V207, Avena sativa, V218I + V260T + A327R + 1340E + L359M |
| SEQ ID NO: 72 | DNA | V207, Apera spica-venti, V218I + V260T + A327R + 1340E + L359M |
| SEQ ID NO: 73 | DNA | V207, Alopecurus myosuroides, V2171 + V260T + A326R + 1339E + L358M |
| SEQ ID NO: 74 | DNA | V208, Avena sativa, V218I + P271N + A327R + 1340E + L359M |
| SEQ ID NO: 75 | DNA | V208, Apera spica-venti, V218I + P271N + A327R + 1340E + L359M |
| SEQ ID NO: 76 | DNA | V208, Alopecurus myosuroides, V218I + P271N + A327R + 1340E + L359M |
| SEQ ID NO: 77 | DNA | V209, Avena sativa, R214G + V218I + V260A + A327R + 1340E + L359M |
| SEQ ID NO: 78 | DNA | V209, Apera spica-venti, R214G + V218I + V260A + A327R + 1340E + L359M |
| SEQ ID NO: 79 | DNA | V209, Alopecurus myosuroides, R214G + V218I + V260A + A327R + 1340E + L359M |
| SEQ ID NO: 80 | DNA | V296, Avena sativa, R214G + V218I + A327R + 1340E + L359Y + G411A |
| SEQ ID NO: 81 | DNA | V296, Apera spica-venti, R214G + V218I + A327R + 1340E + L359Y + G411A |
| SEQ ID NO: 82 | DNA | V296, Alopecurus myosuroides, R214G + V218I + A327R + 1340E + L359Y + G411A |
| SEQ ID NO: 83 | DNA | V300, Avena sativa, R214G + V218I + V260A + A327R + 1340E + L359Y + G411A |
| SEQ ID NO: 84 | DNA | V300, Apera spica-venti, R214G + V218I + V260A + A327R + 1340E + L359Y + G411A |
| SEQ ID NO: 85 | DNA | V300, Alopecurus myosuroides, R214G + V218I + V260A + A327R + 1340E + L359Y + G411A |
| SEQ ID NO: 86 | DNA | V301, Avena sativa, R214G + V218I + V260A + A327R + 1340E + L359M + G411A |
| SEQ ID NO: 87 | DNA | V301, Apera spica-venti, R214G + V218I + V260A + A327R + 1340E + L359M + G411A |
| SEQ ID NO: 88 | DNA | V301, Alopecurus myosuroides, R214G + V218I + V260A + A327R + 1340E + L359M + G411A |
| SEQ ID NO: 89 | DNA | V304, Avena sativa, R214G + V218I + V260A + P271N + A327R + 1340E + L359M + G411A |
| SEQ ID NO: 90 | DNA | V304, Apera spica-venti, R214G + V218I + V260A + P271N + A327R + 1340E + L359M + G411A |
| SEQ ID NO: 91 | DNA | V304, Alopecurus myosuroides, R214G + V218I + V260A + P271N + A327R + 1340E + L359M + G411A |
| SEQ ID NO: 92 | DNA | V306, Avena sativa, R214G + V218I + P271N + A327R + 1340E + L359M |
| SEQ ID NO: 93 | DNA | V306, Apera spica-venti, R214G + V218I + P271N + A327R + 1340E + L359M |
| SEQ ID NO: 94 | DNA | V306, Alopecurus myosuroides, R214G + V218I + P271N + A327R + 1340E + L359M |
| SEQ ID NO: 95 | DNA | V307, Avena sativa, R214G + V218I + V260A + P271N + A327R + 1340E + L359M |
| SEQ ID NO: 96 | DNA | V307, Apera spica-venti, R214G + V218I + V260A + P271N + A327R + 1340E + L359M |
| SEQ ID NO: 97 | DNA | V307, Alopecurus myosuroides, R214G + V218I + V260A + P271N + A327R + 1340E + L359M |
| SEQ ID NO: 98 | DNA | V313, Avena sativa, R214G + V218I + V260A + P271N + A327R + 1340E + L359Y |
| SEQ ID NO: 99 | DNA | V313, Apera spica-venti, R214G + V218I + V260A + P271N + A327R + 1340E + L359Y |
| SEQ ID NO: 100 | DNA | V313, Alopecurus myosuroides, R214G + V218I + V260A + P271N + A327R + 1340E + L359Y |
| SEQ ID NO: 101 | DNA | V316, Avena sativa, R214G + V218I + V260T + A327R + 1340E + L359M + G411A |
| SEQ ID NO: 102 | DNA | V316, Apera spica-venti, R214G + V218I + V260T + A327R + 1340E + L359M + G411A |
| SEQ ID NO: 103 | DNA | V316, Alopecurus myosuroides, R214G + V218I + V260T + A327R + 1340E + L359M + G411A |
| SEQ ID NO: 104 | DNA | V332, Avena sativa, R214G + V218I + V260A + P271N + A327R + 1340E + L359M + K404N |
| SEQ ID NO: 105 | DNA | V332, Apera spica-venti, R214G + V218I + V260A + P271N + A327R + 1340E + L359M + K404N |
| SEQ ID NO: 106 | DNA | V332, Alopecurus myosuroides, R214G + V218I + V260A + P271N + A327R + 1340E + L359M + K404N |
| SEQ ID NO: 107 | DNA | V349, Avena sativa, V218I + P271N + S304T + A327R + 1340E + L359M |
| SEQ ID NO: 108 | DNA | V349, Apera spica-venti, V218I + P271N + S304T + A327R + 1340E + L359M |
| SEQ ID NO: 109 | DNA | V349, Alopecurus myosuroides, V218I + P271N + S304T + A327R + 1340E + L359M |
| SEQ ID NO: 110 | DNA | V351, Avena sativa, R214G + V218I + S304T + A327R + 1340E + L359Y + G411A |
| SEQ ID NO: 111 | DNA | V351, Apera spica-venti, R214G + V218I + S304T + A327R + 1340E + L359Y + G411A |
| SEQ ID NO: 112 | DNA | V351, Alopecurus myosuroides, R214G + V218I + S304T + A327R + 1340E + L359Y + G411A |
| SEQ ID NO: 113 | DNA | V357, Avena sativa, R214G + V218I + P271N + S304T + A327R + 1340E + L359M |
| SEQ ID NO: 114 | DNA | V357, Apera spica-venti, R214G + V218I + P271N + S304T + A327R + 1340E + L359M |
| SEQ ID NO: 115 | DNA | V357, Alopecurus myosuroides, R214G + V218I + P271N + S304T + A327R + 1340E + L359M |
| SEQ ID NO: 116 | DNA | V358, Avena sativa, R214G + V218I + V260A + P271N + S304T + A327R + 1340E + L359M |
| SEQ ID NO: 117 | DNA | V358, Apera spica-venti, R214G + V218I + V260A + P271N + S304T + A327R + 1340E + L359M |
| SEQ ID NO: 118 | DNA | V358, Alopecurus myosuroides, R214G + V218I + V260A + P271N + S304T + A327R + 1340E + L359M |
| SEQ ID NO: 119 | DNA | pBin Vector Avena sativa HPPD V207 |
| SEQ ID NO: 120 | DNA | pBin Vector Avena sativa HPPD V208 |
| SEQ ID NO: 121 | DNA | pBin Vector Avena sativa HPPD V209 |
| SEQ ID NO: 122 | PRT | V162, Avena sativa, V218I + A327R + 1340E + L359M |
| SEQ ID NO: 123 | PRT | V162, Apera spica-venti, V218I + A327R + 1340E + L359M |
| SEQ ID NO: 124 | PRT | V180, Avena sativa, V201, Avena sativa, V218I + A327R + 1340E + L359M + G411A |
| SEQ ID NO: 125 | PRT | V180, Apera spica-venti, V218I + A327R + 1340E + L359M + G411A |
| SEQ ID NO: 126 | PRT | V162, Alopecurus myosuroides, V218I + A327R + 1340E + L359M |
| SEQ ID NO: 127 | PRT | V180, Alopecurus myosuroides, V218I + A327R + 1340E + L359M + G411A |
| SEQ ID NO: 128 | DNA | V162, Avena sativa, V218I + A327R + 1340E + L359M |
| SEQ ID NO: 129 | DNA | V162, Apera spica-venti, V218I + A327R + 1340E + L359M |
| SEQ ID NO: 130 | DNA | V180, Avena sativa, V218I + A327R + 1340E + L359M + G411A |
| SEQ ID NO: 131 | DNA | V180, Apera spica-venti, V218I + A327R + 1340E + L359M + G411A |
| SEQ ID NO: 132 | DNA | V162, Alopecurus myosuroides, V218I + A327R + 1340E + L359M |
| SEQ ID NO: 133 | DNA | V180, Alopecurus myosuroides, V218I + A327R + 1340E + L359M + G411A |
| SEQ ID NOS: | PRT | Native HPPD from different sources |
| 134-188 | | |

What is claimed is:

1. An isolated or recombinant polypeptide comprising an amino acid sequence encoding a 4-hydroxyphenylpyruvate dioxygenase (HPPD) protein that is tolerant to an HPPD inhibitor herbicide compound, wherein said protein comprises:
(a) an amino acid sequence having at least 95% sequence identity to SEQ ID NO: 2, wherein said amino acid sequence comprises a substitution at an amino acid position corresponding to amino acid position 218, position 327, position 340, position 359, and position 411 of SEQ ID NO: 2, wherein the amino acid at position 218 is substituted with an I, the amino acid at position 327 is substituted with an R, the amino acid at position 340 is substituted with an E, the amino acid at position 359 is substituted with an M, and the amino acid at position 411 is substituted with an A;
(b) the amino acid sequence of (a), further comprising a substitution at an amino acid position corresponding to position 260 of SEQ ID NO: 2, wherein the amino acid at position 260 is substituted with an A;
(c) an amino acid sequence haying at least 95% sequence identity to SEQ ID NO: 2, wherein said amino acid sequence comprises a substitution at an amino acid position corresponding to amino acid position 218, position 271, position 327, position 340, and position 359 of SEQ ID NO: 2, wherein the amino acid at position 218 is substituted with an I, the amino acid at position 271 is substituted with an N, the amino acid at position 327 is substituted with an R, the amino acid at position 340 is substituted with an E, and the amino acid at position 359 is substituted with an M;
(d) an amino acid sequence haying at least 95% sequence identity to SEQ ID NO: 2, wherein said amino acid sequence comprises a substitution at an amino acid position corresponding to amino acid position 214, position 218, position 327, position 340, position 359, and position 411 of SEQ ID NO: 2, wherein the amino acid at position 214 is substituted with a G, the amino acid at position 218 is substituted with an I, the amino acid at position 327 is substituted with an R, the amino acid at position 340 is substituted with an E, the amino acid at position 359 is substituted with a Y, and the amino acid at position 411 is substituted with an A;
(e) the amino acid sequence of (d), further comprising a substitution at an amino acid position corresponding to position 304 of SEQ ID NO: 2, wherein the amino acid at position 304 is substituted with a T;
(f) an amino acid sequence haying at least 95% sequence identity to SEQ ID NO: 1, wherein said amino acid sequence comprises a substitution at an amino acid position corresponding to amino acid position 218, position 271, position 327, position 340, and position 359 of SEQ ID NO: 1, wherein the amino acid at position 218 is substituted with an I, the amino acid at position 271 is substituted with an N, the amino acid at position 327 is substituted with an R, the amino acid at position 340 is substituted with an E, and the amino acid at position 359 is substituted with an M;
(g) an amino acid sequence haying at least 95% sequence identity to SEQ ID NO: 1, wherein said amino acid sequence comprises a substitution at an amino acid position corresponding to amino acid position 214, position 218, position 327, position 340, position 359, and Position 411 of SEQ ID NO: 1, wherein the amino acid at position 214 is substituted with a G, the amino acid at position 218 is substituted with an I, the amino acid at position 327 is substituted with an R, the amino acid at position 340 is substituted with an E, the amino acid at position 359 is substituted with a Y, and the amino acid at position 411 is substituted with an A; or
(h) the amino acid sequence of (q), further comprising a substitution at an amino acid position corresponding to position 304 of SEQ ID NO: 1, wherein the amino acid at position 304 is substituted with a T.

2. An isolated or recombinant polynucleotide encoding the polypeptide of claim 1.

3. (canceled)

4. The isolated or recombinant polynucleotide of claim 2, wherein the nucleotide sequence of the isolated polynucleotide is optimized for expression in a plant.

5. The isolated or recombinant polynucleotide of claim 2, wherein said polynucleotide is operably linked to a promoter.

6. The isolated or recombinant polynucleotide of claim 5, wherein the promoter drives expression in a plant or plant cell.

7. An expression cassette comprising the isolated polynucleotide of claim 5.

8. The expression cassette of claim 7, further comprising a recombinant or isolated polynucleotide sequence encoding a polypeptide that confers a desirable trait.

9. The expression cassette of claim 8, wherein the desirable trait is resistance to an herbicide.

10-15. (canceled)

16. A cell comprising a heterologous polynucleotide encoding the polypeptide of claim 1.

17. The cell of claim 16, wherein said cell is a plant cell.

18. A plant or plant part having stably integrated into its genome a heterologous polynucleotide encoding the polypeptide of claim 1, wherein expression of said polypeptide in the plant confers the with plant increased herbicide tolerance as compared to a control plant.

19. The plant or plant part of claim 18, wherein said plant has stably incorporated into its genome, an additional heterologous polynucleotide encoding a polypeptide that confers resistance to another herbicide.

20. The plant or plant part of claim 18, wherein said polynucleotide encoding said heterologous polypeptide has been introduced into the plant or plant part by transformation.

21-22. (canceled)

23. The plant or plant part of claim 18, wherein said plant is a monocot.

24. The plant or plant part of claim 23, wherein said monocot is corn, rye, barley, rice, sorghum, oat, sorghum, sugarcane, switch grass, miscanthus grass, or wheat

25. The plant or plant part of claim 18, wherein said plant is a dicot.

26. The plant or plant part of claim 25, wherein said dicot is soybean, sunflower, tomato, sugarbeet, tobacco, a cole crop, potato, sweet potato, cassava, safflower, trees, alfalfa, pea, and cotton.

27. A seed produced by the plant of claim 18, wherein said seed has stably incorporated into its genome a heterologous polynucleotide encoding the polypeptide of claim 1.

28. (canceled)

29. A method for conferring resistance to an HPPD inhibitor in a plant, the method comprising introducing a heterologous polynucleotide encoding a polypeptide of claim 1 into the plant.

30. A method of controlling undesired vegetation in an area of cultivation, the method comprising
a) providing, at said area of cultivation, a plant of claim 18; and
b) applying to said area of cultivation, an effective amount of an HPPD inhibitor compound.

31. The method of claim 30, wherein the plant comprises at least one additional heterologous nucleic acid comprising a nucleotide sequence encoding a herbicide tolerance enzyme.

32-34. (canceled)

35. A method of identifying or selecting a transformed plant cell, plant tissue, plant or part thereof comprising:
i) providing a transformed plant or plant part thereof, wherein said transformed plant or plant part comprises a polynucleotide encoding a polypeptide of claim 1 operably linked to a promoter that drives expression the plant or plant part;
ii) contacting the transformed plant or plant part with at least one HPPD inhibitor compound;
iii) determining whether the plant or plant part is affected by the HPPD inhibiting compound; and
iv) identifying or selecting the transformed plant or plant part having said polynucleotide.

36. A method for growing a plant of claim 18 while controlling weeds in the vicinity of said plant, said method comprising the steps of:
a) growing said plant; and
b) applying an effective amount of a herbicide composition comprising an HPPD inhibitor to the plant and weeds.

37-45. (canceled)
